SEQUENCE LISTING (116 sequences)

SEQ ID NO: 1 - protein, 410 aa, Drosophila melanogaster
MDTLRALEPLHRACQVCNLWPWRLAPPPDSEGILLRRSRWLELYGWTVLIAATSFTVYGLFQESSVEEKQDSESTISSIGHTVDFIQLVGMRVAHLAALLEALWQRQAQRGFFAELGEIDRLLSKALRVDVEAMRINMRRQTSRRAVWILWGYAVSQLLILGAKLLSRGDRFPIYWISYLLPLLVCGLRYFQIFNATQLVRQRLDVLLVALQQLQLHQKGPAVDTVLEEQEDLEEAAMDRLIAVRLVYQRVWALVALLNRCYGLSMLMQVGNDFLAITSNCYWMFLNFRQSAASPFDILQIVASGVWSAPHLGNVLVLSLLCDRTAQCASRLALCLHQVSVDLRNESHNALITQFSLQLLHQRLHFSAAGFFNVDCTLLYTIVGATTTYLIILIQFHMSESTIGSDSNGQ

SEQ ID NO: 2 - protein, 385 aa, Drosophila melanogaster
MSGHLGRVLQFHLRLYQVLGFHGLPLPGDGNPARTRRRLMAWSLFLLISLSALVLACLFSGEEFLYRGDMFGCANDALKYVFAELGVLAIYLETLSSQRHLANFWWLHFKLGGQKTGLVSLRSEFQQFCRYLIFLYAMMAAEVAIHLGLWQFQALTQHMLLFWSTYEPLVWLTYLRNLQFVLHLELLREQLTGLEREMGLLAEYSRFASETGRSFPGFESFLRRRLVQKQRIYSHVYDMLKCFQGAFNFSILAVLLTINIRIAVDCYFMYYSIYNNVINNDYYLIVPALLEIPAFIYASQSCMVVVPRIAHQLHNIVTDSGCCSCPDLSLQIQNFSLQLLHQPIRIDCLGLTILDCSLLTRMACSVGTYMIYSIQFIPKFSNTYM

SEQ ID NO: 3 - protein, 381 aa, Drosophila melanogaster
MQRTHLEFEFKNAPQEPKRPFEFFMYFKFCLINLMMMIQVCGIFAQYGEVGKGSVSQVRVHFAIYAFVLWNYTENMADYCYFINGSVLKYYRQFNLQLGSLRDEMDGLRPGGMLLHHCCELSDRLEELRRRCREIHDLQRESFRMHQFQLIGLMLSTLINNLTNFYTLFHMLAKQSLEEVSYPVVVGSVYATGFYIDTYIVALINEHIKLELEAVALTMRRFAEPREMDERLTREVRNKIFSFLATTLEIMIQIWLSFFANFDDVTPYRKCENRPKNLFFKIRQKVIGIVSSGKLKLLVSLRFFIIDNRLILNLHKYLAIKLNFLNLIQIEHLSLELLNYQPPMLCGLLHLDRRLVYLIAVTAFSYFITLVQFDLYLRKKS

SEQ ID NO: 4 - protein, 373 aa, Drosophila melanogaster
MRVGKLCRLALRFWMGLILVLGFSSHYYNPTRRRLVYSRILQTYDWLLMVINLGAFYLYYRYAMTYFLEGMFRRQGFVNQVSTCNVFQQLLMAVTGTWLHFLFERHVCQTYNELSRILKHDLKLKEHSRFYCLAFLAKVYNFFHNFNFALSAIMHWGLRPFNVWDLLANLYFVYNSLARDAILVAYVLLLLNLSEALRLNGQQEHDTYSDLMKQLRRRERLLRIGRRVHRMFAWLVAIALIYLVFFNTATIYLGYTMFIQKHDALGLRGRGLKMLLTVVSFLVILWDVVLLQVICEKLLAEENKICDCPEDVASSRTTYRQWEMSALRRAITRSSPENNVLGMFRMDMRCAFALISCSLSYGIIIIQIGYIPG

SEQ ID NO: 5 - protein, 431 aa, Drosophila melanogaster
MAFKLWERFSQADNVFQALRPLTFISLLGLAPFRLNLNPRKEVQTSKFSFFAGIVHFLFFVLCFGISVKEGDSIIGYFFQTNITRFSDGTLRLTGILAMSTIFGFAMFKRQRLVSIIQNNIVVDEIFVRLGMKLDYRRILLSSFLISLGMLLFNVIYLCVSYSLLVSATISPSFVTFTTFALPHINISLMVFKFLCTTDLARSRFSMLNEILQDILDAHIEQLSALELSPMHSVVNHRRYSHRLRNLISTPMKRYSVTSVIRLNPEYAIKQVSNIHNLLCDICQTIEEYFTYPLLGIIAISFLFILFDDFYILEAILNPKRLDVFEADEFFAFFLMQLIWYIVIIVLIVEGSSRTILHSSYTAAIVHKILNITDDPELRDRLFRLSLQLSHRKVLFTAAGLFRLDRTLIFTVNFLQITGAATCYLIILIQF

SEQ ID NO: 6 - protein, 415 aa, Drosophila melanogaster
MIRCGLDIFRGCRGRFRYWLSARDCYDSISLMVAIAFALGITPFLVRRNALGENSLEQSWYGFLNAIFRWLLLAYCYSYINLRNESLIGYFMRNHVSQISTRVHDVGGIIAAVFTFILPLLLRKYFLKSVKNMVQVDTQLERLRSPVNFNTVVGQVVLVILAVVLLDTVLLTTGLVCLAKMEVYASWQLTFIFVYELLAISITICMFCLMTRTVQRRITCLHKFDFATMSALRRVRKYFISSQVYEALRPLFFLTFLYGLTPFHVVRRKMGESYLKMSCFGVFNIFIYICLCGFCYISSLRQGESIVGYFFRTEISTIGDRLQIFNGLIAGAVIYTSAILKRCKLLGTLTILHSLDTNFSNIGVRVKYSRIFRYSLLVLIFKLLILGVYFVGVFRLLVSLDVTPSFCVCMTFFLQ

SEQ ID NO: 7 - protein, 472 aa, Drosophila melanogaster
MKRKAVEVIGLIPLNRQQSETNFILDYAMMCIVPIFYVACYLLINLSHIIGLCLLDSCNSVCKLSSHLFMHLGAFLYLTITLLSLYRRKEFFQQFDARLNDIDAVIQKCQRVAEMDKVKVTAVKHSVAYHFTWLFLFCVFTFALYYDVRSLYLTFGNLAFIPFMVSSFPYLAGSIIQGEFIYHVSVISQRFEQINMLLEKINQEARHRHAPLTVFDIESEGKKERKTVTPITVMDGRTTTGFGNENKFAGEMKRQEGQQKNDDDDLDTSNDEDEDDFDYDNATIAENTGNTSEANLPDLFKLHDKILALSVITNGEFGPQCVPYMAACFVVSIFGIFLETKVNFIVGGKSRLLDYMTYLYVIWSFTTMMVAYIVLRLCCNANNHSKQSAMIVHEIMQKKPAFMLSNDLFYNKMKSFTLQFLHWEGFFQFNGVGLFALDYTFIFSTVSAATSYLIVLLQFDMTAILRNEGLMS

SEQ ID NO: 8 - protein, 390 aa, Drosophila melanogaster
MVDWVVLLLKAVHIYCYLIGLSNFEFDCRTGRVFKSRRCTIYAFMANIFILITIIYNFTAHGDTNLLFQSANKLHEYVIIIMSGLKIVALITVLNRWLQRGQMMQLVKDVIRLYMINPQLKSMIRWGILLKAFISFAIELLQVTLSVDALDRQGTAEMMGLLVKLCVSFIMNLAISQHFLVILLIRAQYRIMNAKLRMVIEESRRLSFLQLRNGAFMTRCCYLSDQLEDIGEVQSQLQSMVGQLDEVFGMQGLMAYSEYYLSIVGTSYMSYSIYKYGPHNLKLSAKTSIIVCILITLFYLDALVNCNNMLRVLDHHKDFLGLLEERTVFASSLDIRLEESVSFESLQLQLARNPLKINVMGMFPITRGSTAAMCASVIVNSIFLIQFDME

SEQ ID NO: 9 - protein, 344 aa, Drosophila melanogaster
MDLESFLLGAVYYYGLFIGLSNFEFDWNTGRVFTKKWSTLYAIALDSCIFALYIYHWTGNTNIVNAIFGRANMLHEYVVAILTGLRIVTGLFTLILRWYQRCKMMDLASKVVRMYVARPQVRRMSRWGILTKFIFGSITDGLQMAMVLSAMGSRVDSQFYLGLGLQYWMFVILNMAMMQQHMIMLFVRTQFQLINTELRQVIDEAKDLLLSPRHQGVFMTKCCSLADQIENIARIQSQLQTIMNQMEEVFGIQGAMTYGGYYLSSVGTCYLAYSILKHGYENLSMTLSTVILAYSWCFFYYLDGMLNLSVMLHVQDDYWEMLQILGKRTIFVGLDVRLEEAVST

SEQ ID NO: 10 - protein, 383 aa, Drosophila melanogaster
MIKLYFRYSLAIGITSQQFSNRKFFSTLFSRTYALIANIVTLIMLPIVMWQVQLVFQQKKTFPKLILITNNVREAVSFLVILYTVLSRGFRDTAFKEMQPLLLTLFREEKRCGFKGIGGVRRSLRILLFVKFFTLSWLCVTDVLFLLYSTDALIWVNVLRFFFKCNTNNILEMVPMGYFLALWHIARGFDCVNRRLDQIVKSKSTRKHRELQHLWLLHACLTKTALNINKIYAPQMLASRFDNFVNGVIQAYWGAVFTFDLSTPFFWVVYGSVQYHVRCLDYYLIDNMCDVAVEYHDSAKHSWSEVRWTKEVSAFGSILLYICMLMQLLSFQISSYVIYANSTKLQLWSCGLFQANRSMWFAMISSVLYYILVLLQFHLVMRK

SEQ ID NO: 11 - protein, 436 aa, Drosophila melanogaster
MSRTSDDIRKHLKVRRQKQRAILAMRWRCAQGGLEFEQLDTFYGAIRPYLCVAQFFGIMPLSNIRSRDPQDVKFKVRSIGLAVTGLFLLLGGMKTLVGANILFTEGLNAKNIVGLVFLIVGMVNWLNFVGFARSWSHIMLPWSSVDILMLFPPYKRGKRSLRSKVNVLALSVVVLAVGDHMLYYASGYCSYSMHILQCHTNHSRITFGLYLEKEFSDIMFIMPFNIFSMCYGFWLNGAFTFLWNFMDIFIVMTSIGLAQRFQQFAARVGALEGRHVPEALWYDIRRDHIRLCELASLVEASMSNIVFVSCANNVYVICNQALAIFTKLRHPINYVYFWYSLIFLLARTSLVFMTASKIHDASLLPLRSLYLVPSDGWTQEVQRFADQLTSEFVGLSGYRLFCLTRKSLFGMLATLVTYELMLLQIDAKSHKGLRCA

SEQ ID NO: 12 - protein, 512 aa, Drosophila melanogaster [sequence omitted]

SEQ ID NO: 13 - protein, 367 aa, Drosophila melanogaster
MPVRKVSSKFAEDLTFTWFSVRSYYALVTILFFGVSSGYMVAFVTSVSFNFDSVETLVFYLSIFLISLSFFQLARKWPEIAQSWQLVEAKLPPLKLPKERRSLAQHINMITIVATTCSLVEHIMSMLSMGYYVNSCPRWPDRPIDSFLYLSFSSVFYFVDYTRFLGIVGKVVNVLSTFAWNFNDIFVMAVSVALAARFRQLNDYMMREARLPTTVDYWMQCRINFRNLCKLCEEVDDAISTITLLCFSNNLYFICGKILKSMQAKPSIWHALYFWFSLVYLLGRTLILSLYSSSINDESKRPLVIFRLVPREYWCDELKRFSEEVQMDNVALTGMKFFRLTRGVVISVAGTIVTYELILLQFNGEEK

SEQ ID NO: 14 - protein, 409 aa, Drosophila melanogaster
MELSRSDKEAFLSDGSFHQAVGRVLLVAEFFAMMPVKGVTGKHPSDLSFSWRNIRTCFSLLFIASSLANFGLSLFKVLNNPISFNSIKPIIFRGSVLLVLIVALNLARQWPQLMMYWHTVEKDLPQYKTQLTKWKMGHTISMVMLLGMMLSFAEHILSMVSAINYASFCNRTADPIQNYFLRTNDEIFFVTSYSTTLALWGKFQNVFSTFIWNYMDLFVMIVSIGLASKFRQLNDDLRNFKGMNMAPSYWSERRIQYRNICILCDKMDDAISLITMVSFSNNLYFICVQLLRSLNTMPSVAHAVYFYFSLIFLIGRTLAVSLYSSSVHDESRLTLRYLRCVPKESWCPEVKRFTEEVISDEVALTGMKFFHLTRKLVLSVAGTIVTYELVLIQFHEDNDLWDCDQSYYS

SEQ ID NO: 15 - protein, 498 aa, Drosophila melanogaster
MDNMAQAEDAVQPLLQQFQQLFFISKIAGILPQDLEKFRSRNLLEKSRNGMIYMLSTLILYVVLYNILIYSFGEEDRSLKASQSTLTFVIGLFLTYIGLIMMVSDQLTALRNQGRIGELYERIRLVDERLYKEGCVMDNSTIGRRIRIMLIMTVIFELSILVSTYVKLVDYSQWMSLLWIVSAIPTFINTLDKIWFAVSLYALKERFEAINATLEELVDTHEKHKLWLRGNQEVPPPLDSSQPPQYDSNLEYLYKELGAIDAASRKPPPPPLATNMVHESELGNAAKVEEKLNNLCQVHDEICEIGKALNELWSYPILSLMAYGFLIFTAQLYFLYCATQYQSIPSLFRSAKNPFITVIVLSYTSGKCVYLIYLSWKTSQASKRTGISLHKCGVVADDNLLYEIVNHLSLKLLNHSVDFSACGFFTLDMETLYGVSGGITSYLIILIQFNLAAQQAKEAIQTFNSLNDTAGLVGAATDMDNISSTLRDFVTTTMTPAV

SEQ ID NO: 16 - protein, 346 aa, Drosophila melanogaster
MFEFLHQMSAPKLSTSILRYIFRYAQFIGVIFFCLHTRKDDKTVFIRNWLKWLNVTHRIITFTRFFWVYIASISIKTNRVLQVLHGMRLVLSIPNVAVILCYHIFRGPEIIDLINQFLRLFRQVSDLFKTKTPGFGGRRELILILLNLISFAHEQTYLWFTIRKGFSWRFLIDWWCDFYLVSATNIFIHINSIGYLSLGVLYSELNKYVYTNLRIQLQKLNTSGSKQKIRRVQNRLEKCISLYREIYHTSIMFHKLFVPLLFLALIYKVLLIALIGFNVAVEFYLNSFIFWILLGKHVLDLFLVTVSVEGAVNQFLNIGMQFGNVGDLSKFQTTVSQFIFIDFIPI

SEQ ID NO: 17 - protein, 736 aa, Drosophila melanogaster
MVAQKSRLLARAFPYLDIFSVFALTPPPQSFGHTPHRRLRWYLMTGYVFYATAILATVFIVSYFNIIAIDEEVLEYNVSDFTRVMGNIQKSLYSIMAIANHLNMLINYRRLGGIYKDIADLEMDMDEASQCFGGQRQRFSFRFRMALCVGVWMILMVGSMPRLTMTAMGPFVSTLLKILTEFVMIMQQLKSLEYCVFVLIIYELVLRLRRTLSQLQEEFQDCEQQDMLQALCVALKRNQLLLGRIWRLEGDVGSYFTPTMLLLFLYNGLTILHMVNWAYINKFLYDSCCQYGPEYCLFVLLVYELILRTRHVLEQLKDDLEDFDCGARIQELCVTLKQNQLLIGRIWRLVDEIGAYFRWSMTLLFLYNGLTILHVVNWAIIRSIDPNDCCQLMSFHFSLNMEANRSRLLAAARPYIQIYSIFGLTPPIQFFTRTLHKRRRGIVILGYACYLISISLMVIYECYANIVALQKDIHKFHAEDSSKVMGNTQKVLVVAMFVWNQLNILLNFRRLARIYDDIADLEIDLNNASSGFVGQRHWWRFRFRLALSVGLWIVLLVGLTPRFTLVALGPYLHWTNKVLTEIILIMLQLKCTEYCVFVLLIYELILRGRHILQQISVELEGNQSRDSVQELCVALKRNQLLAGRIWGLVNEVSLYFTLSLTLLFLYNELTILQIVNWALIKSVNPNECCQYTEDYLILKMGLREYSLQMEHLKLIFTCGGLFDINLKFFGGVKLKL

SEQ ID NO: 18 - protein, 294 aa, Drosophila melanogaster
MEAKRSRLLTTARPYLQVLSLFGLTPPAEFFTRTLRKRRRFCWMAGYSLYLIAILLMVFYEFHANIVSLHLEIYKFHVEDFSKVMGRTQKFLIVAIATCNQLNILLNYGRLGLIYDEIANLDLGIDKSSKNFCGKSHWWSFRLRLTLSIGLWMVIIIGVIPRLTLGRAGPFFHWVNQVLTQIILIMLQLKGPEYCLFVLLVYELILRTRHVLEQLKDDLEDFDCGARIQELCVTLKQNQLLIGRIWRLVDEIGAYFRWSMTLLFLYNGLTILHVVNWAIIRSIDPNDCCQLSEE

SEQ ID NO: 19 - protein, 398 aa, Drosophila melanogaster
MFRPSGSGYRQKWTGLTLKGALYGSWILGVFPFAYDSWTRTLRRSKWLIAYGFVLNAAFILLVVTNDTESETPLRMEVFHRNALAEQINGIHDIQSLSMVSIMLLRSFWKSGDIERTLNELEDLQHRYFRNYSLEECISFDRFVLYKGFSVVLELVSMLVLELGMSPNYSAQFFIGLGSLCLMLLAVLLGASHFHLAVVFVYRYVWIVNRELLKLVNKMAIGETVESERMDLLLYLYHRLLDLGQRLASIYDYQMVMVMVSFLIANVLGIYFFIIYSISLNKSLDFKILVFVQALVINMLDFWLNVEICELAERTGRQTSTILKLFNDIENIDEKLERSVSFTSQHYCETDFALFCSHRRLRFHHCGLFYVNYEMGFRMAITSFLYLLFLIQFDYWNL

SEQ ID NO: 20 - protein, 320 aa, Drosophila melanogaster
MVKQAEDREHGIMLDVFQRNALLYQISSLMGVVGVVSICTVHLRTLWRSKHLEEIYNGLMLLEAKYFCSNAVECPAFDGYVIQKGVVIVVGLLAPWMVHFGMPDSKLPVLNVLVVSMVKLGTLLLALHYHLGVVIIYRFVWLINRELLSLVCSLRGNHKGSSSRVRFLLKLYNKLVNLYSKLADCYDCQTVLMMAIFLAANIIVCFYMIVYRISLSKMSFFVMLIMFPLAIANNFMDFWLSMKVCDLLQKTGRQTSMILKLFNDIENMDKDLEISISDFALYCSHRRFKFLHCGLFHVNREMGFKMFVASVLYLLYLVQF

SEQ ID NO: 21 - protein, 389 aa, Drosophila melanogaster
MFASRSDLQSRLCWIILKATLYSSWFLGVFPYRFDSRNGQLKRSRFLLFYGLILNFFLLLKMVCSGGQKLGIPEAFARNSVLENTHYTTGMLAVFSCVVIHFLNFWGSTRVQDLANELLVLEYQQFASLNETKCPKFNSFVIQKWLSVIGLLLSYLSIAYGLPGNNFSVEMVLINSLVQFSFNCNIMHYYIGVLLIYRYLWLINGQLLEMVTNLKLDCSVDSSRIRKYLSLYRRLLELKGYMVATYEYHMTLVLTTGLASNFLAIYSWIVLDISMNINFIYLLIFPLFLLVNVWNLWLSIAASDLAENAGKSTQTVLKLFADLEVKDIELERSVSVNSNRYKQVNEFALLCGHCQFNFHVCGLFTINYKMGFQMIITSFLYLIYMIQFD

SEQ ID NO: 22 - protein, 287 aa, Drosophila melanogaster
MINVVIGIINVLSALIVHFMNFWGSRKVGEICNELLILEYQDFEGLNGRNCPNFNCFVIQKCLTILGQLLSFFTLNFALPGLEFHICLVLLSCLMEFSLNLNIMHYHVGVLLIYRYVWLINEQLKDLVSQLKLNPETDFSRIHQFLSLYKRLLELNRKLVIAYEYQMTLFIIAQLSGNIVVIYFLIVYGLSMRTYSIFLVAFPNSLLINIWDFWLCIAACDLTEKAGDETAIILKIFSDLEHRDDKLEKFRFQLCGLFSMNCRMGFKMIITTFLYLVYLVQFDYMNL

SEQ ID NO: 23 - protein, 410 aa, Drosophila melanogaster
MSQPKRIHRICKGLARFTIRATLYGSWVLGLFPFTFDSRKRRLNRSKWLLAYGLVLNLTLLVLSMLPSTDDHNSVKVEVFQRNPLVKQVEELVEVISLITTLVTHLRTFSRSSELVEILNELLVLDKNHFSKLMLSECHTFNRYVIEKGLVIILEIGSSLVLYFGIPNSKIVVYEAVCIYIVQLEVLMVVMHFHLAVIYIYRYLWIINGQLLDMASRLRRGDSVDPDRIQLLLWLYSRLLDLNHRLTAIYDIQVTLFMATLFSVNIIVGHVLVICWINITRFSLLVIFLLFPQALIINFWDLWQGIAFCDLAESTGKKTSMILKLFNDMENMDQETERRVSEYMFQNLMYFKYFKHPLIFVAEFTLFCSHRRLKVCHLGLLDINYEMGFRMIITNILYVVFLVQFDYMNL

SEQ ID NO: 24 - protein, 364 aa, Drosophila melanogaster
MGVMPIHRNPPEKNLPRTGYSWGSKQVMWAIFIYSCQTTIVVLVLRERVKKFVTSPDKRFDEAIYNVIFISLLFTNFLLPVASWRHGPQVAIFKNMWTNYQYKFFKTTGSPIVFPNLYPLTWSLCVFSWLLSIAINLSQYFLQPDFRLWYTFAYYPIIAMLNCFCSLWYINCNAFGTASRALSDALQTTIRGEKPAQKLTEYRHLWVDLSHMMQQLGRAYSNMYGMYCLVIFFTTIIATYGSISEIIDHGATYKEVGLFVIVFYCMGLLYIICNEAHYASRKVGLDFQTKLLNINLTAVDAATQKEVEMLLVAINKNPPIMNLDGYANINRELITTNISFMATYLVVLLQFKITEQRRIGQQQA

SEQ ID NO: 25 - protein, 377 aa, Drosophila melanogaster
MFQPRRGFSCHLAWFMLQTTLYASWLLGLFPFTFDSRRKQLKRSRWLLLYGFVLHSLAMCLAMSSHLASKQRRKYNAFERNPLLEKIYMQFQVTTFFTISVLLLMNVWKSNTVRKIANELLTLEGQVKDLLTLKNCPNFNCFVIKKHVAAIGQFVISIYFCLCQENSYPKILKILCCLPSVGLQLIIMHFHTEIILVYRYVWLVNETLEDSHHLSSSRIHALASLYDRLLKLSELVVACNDLQLILMLIIYLIGNTVQIFFLIVLGVSMNKRYIYLVASPQLIINFWDFWLNIVVCDLAGKCGDQTSKVLKLFTDLEHDDEELERSLNEFAWLCTHRKFRFQLCGLFSINHNMGFQMIITSFLYLVYLLQFDFMNLC

SEQ ID NO: 26 - protein, 370 aa, Drosophila melanogaster
MKTLECLTRRFLEVIFSVLALVPLPPISQLGWLFLSLAIRCCWIVYFIYLLDVAISFSWVAIENVGNAVGTMLFVGNSVLGFALLLESVLKQKTHSQLEDLRVQTELQLQRLGMFGRSRHAAYLLPLIGVQFTCDLVRLATNFGETVSPVFCISLPLMWLLRYRYVQLVQHVMDLNQRSIHLRRSLLSMASGNDLWQPYGVQECLQLQTLRTTYERIFECYETFSDCYGWGMLGLHLLTSFQFVTNAYWMIMGIYDGGNVRSLIFNGATGIDFGTPIATLFWHGDSGAENGRQIGCLISKLVKPQGSKLYNDLVSEFSLQTLHQRFVVTAKDFFSLNLHLLSSMFAAVVTYLVILIQFMFAERSSTRGSG

SEQ ID NO: 27 - protein, 374 aa, Drosophila melanogaster
MFPPTRVQASSRVVLKIFHFILVAFSLRSRRLSRLVLWLQFLGWLTWFISMWTQSVIYAQTIDCTLDCSLRHILTFFQTVSHAFIVVTSFLDGFRIKQDQLDEPIAFEDSDPWLAFTVLAMLVPTLGVEYLVCSNAPEYAFRIRIYHLKTLPSFLALQVQIISFILEVMKVNIRVRQTKLQLLILARELSCRWPQRKQKPQFSDQQAHRVKDLKRRYNDLHYLFVRINGYFGGSLLTIIIVHFAIFVSNSYWLFVDIRTRPWRIYAILLNLGFIFNVALQMAAACWHCQQSYNLGRQIGCLISKLVKPQGSKLYNDLVSEFSLQTLHQRFVVTAKDFFSLNLHLLSSMFAAVVTYLVILIQFMFAERSSTRGSG

SEQ ID NO: 28 - protein, 416 aa, Drosophila melanogaster
MPIYEQVSDYEVGPPTKTNEFYSFFVRGVVHALTIFNVYSLFTPISAQLFFSYRETDNVNQWIELLLCILTYTLTVFVCAHNTTSMLRIMNEILQLDEEVRRQFGANLSQNFGFLVKFLVGITACQAYIIVLKIYAVQGEITPTSYILLAFYGIQNGLTATYIVFASALLRIVYIRFHFINQLLNGYTYGQQHRRKEGGARARRQRGDVNPNVNPALMEHFPEDSLFIYRMHNKLLRIYKGINDCCNLILVSFLGYSFYTVTTNCYNLFVQITGKGMVSPNILQWCFAWLCLHVSLLALLSRSCGLTTTEVSNYIGDKISIFMSVFISRPMPHPKFLQGCMPSRRSIRISGFHYQIDKFLTKSIKQEVQFTAYGFFAIDNSTLFKIFSAVTTYLVILIQFKQLEDSKVEDPVPEQT

SEQ ID NO: 29 - protein, 369 aa, Drosophila melanogaster
MLYSFHPYLKYFALLGLVPWSESCAQSKFVQKVYSAILIILNAVHFGISIYFPQSAELFLSLMVNVIVFVARIVCVTVIILQVMVHYDDYFRFCREMKYLGLRLQCELKIHVGRLKWQSYAKILALGIGFLVTVLPSIYVALSGSLLYFWSSLLSILIIRMQFVLVLLNVELLGHHVSLLGIRLQNVLECHLMGANCTLDGNANRLCSLEFLLALKQSHMQLHYLFTHFNDLFGWSILGTYVVLFSDSTVNIYWTQQVLVEVYEYKYLYATFSVFVPSFFNILVFCRCGEFCQRQSVLIGSYLRNLSCHPSIGRETSYKDLLMEFILQVEQNVLAINAEGFMSTDNSLLMSILAAKVTYLIVLMQFSSV

SEQ ID NO: 30 - protein, 372 aa, Drosophila melanogaster
MGTRNRKLLFFLHYQRYLGLTNLDFSKSLHIYWLHGTWSSTAIQIVVVGVFMAALLGALAESLYYMETKSQTGNTFDNAVILTTSVTQLLANLWLRSQQKSQVNLLQRLSQVVELLQFEPYAVPQFRWLYRIWLLVCLIYGAMVTHFGINWLTTMQISRVLTLIGFVYRCVLANFQFTCYTGMVVILKKLLQVQVKQLEHLVSTTTISMAGVAGCLRTHDEILLLGQRELIAVYGGVILFLFIYQVMQCILIFYISNLEGFHSSNDLVLIFCWLAPMLFYLILPLVVNDIHNQANKTAKMLTKVPRTGTGLDRMIEKFLLKNLRQKPILTAYGFFALDKSTLFKLFTAIFTYMVILVQFKEMENSTKSINKF

SEQ ID NO: 31 - protein, 381 aa, Drosophila melanogaster
MDFQPGELCAYYRLCRYLGIFCIDYNPTKKKFRLRRSVLCYIVHFALQAYLVGCISVMVTYWRRCFKSELTTTGNHFDRLVMVIALGILVVQNAWLIWLQAPHLRIVRQIEFYRRNHLANVRLLLPKRLLWLIIATNVVYMANFIKTCIFEWLTDASRLFVITSLGFPLRYLVTSFTMGTYFCMVHIVRLVLDWNQSQINAIIDESADLKMTSPNRLRLRVCLEMHDRLMLLCNDEISLVYGFIAWLSWMFASLDVTGVIYLTMVIQTKKSIVLKLITNVVWLSPTFMTCAASFMSNRVTIQANKTAKMLTKVPRTGTGLDRMIEKFLLKNLRQKPILTAYGFFALDKSTLFKLFTAIFTYMVILVQFKEMENSTKSINKF

SEQ ID NO: 32 - protein, 381 aa, Drosophila melanogaster
MKRNAFEELRVQLRTLKWLGVLRFTIDFNKCLVRENASEERSAWLYLIGVVGITCSLIVYSTYFPSHFIMGKHNTTGNCYALINIRSCSIVTMLIYTQLYIQRFRFVALLQSILRFNQISGSHREEGRFAFYYYTHLSLLIICMLNYAYGYWTAGVRLTTIPIYLLQYGFSYLFLGQVVVLFACIQQILLSILKYYNQVVLKNIKSSKESREFYYNFCKYNQVIWLSYTEINHCFGLLLLLVTGLILLITPSGPFYLVSTIFEGRFRQNWQFSLMSFTAILWSLPWIVLLVLAMGRNDVQKEANKTAKMLTKVPRTGTGLDRMIEKFLLKNLRQKPILTAYGFFALDKSTLFKLFTAIFTYMVILVQFKEMENSTKSINKF

SEQ ID NO: 33 - protein, 371 aa, Drosophila melanogaster
MSKVCRDLRIYLRLLHIMGMMCWHFDSDHCQLVATSGSERYAVVYAGCILVSTTAGFIFALLHPSRFHIAIYNQTGNFYEAVIFRSTCVVLFLVYVILYAWRHRYRDLVQHILRLNRRCASSCTNQQFLHNIILYGMLTILCFGNYLHGYTRAGLATLPLALCMLVYIFAFLVLCLLLMFFVSLKQVMTAGLIHYNQQLCQGDLISGLRGRQQILKLCGGELNECFGLLMLPIVALVLLMAPSGPFFLISTVLEGKFRPDECLIMLLTSSTWDTPWMIMLVLMLRTNGISEEANKTAKMLTKVPRTGTGLDRMIEKFLLKNLRQKPILTAYGFFALDKSTLFKLFTAIFTYMVILVQFKEMENSTKSINKF

SEQ ID NO: 34 - protein, 379 aa, Drosophila melanogaster
MKSATSKVVTALDVSVVVMAIVSGVYCGLFSLNDTLELNDRLNKIDNTLNAYNNFRRDRWRALGMAAVSLLAISILVGLDVGTWMRIAQDMNIAQSDTELNVHWYIPFYSLYFILTGLQVNIANTAYGLGRRFGRLNRMLSSSFLAENNATSAIKPQKVSTVKNVSVNRPAMPSALHASLTKLNGETLPSEAAGDKAAARSLILNVELLKLGYFPAKNKGLLLKSLADSHESLGKCVHLLSNSFGIAVLFILVSCLLHLVATAYFLFLELLSKRDNGYLWVQMLWICFHFLRLLMVVEPCHLAARESRKTIQIVCEIERKVHEPILAEAVKKFWQQLLVVDADFSACGLCRVNRTILTSFASAIATYLVILIQFQRTNG

SEQ ID NO: 35 - protein, 361 aa, Drosophila melanogaster
MAFTSSQLCSLLTKFTALNGLNTYYFDTKTNAFRVSSKLKIYCAIHHALCVLALAHMSYSTASNLRVSVTVLTIGGTMACCVKSCWEKAQGIRNLARGLVTMEQKYFAGRPSGLLLKCRYYIKITFGSITLLRIHLIQPIYMRRLLPSQFYLNVGAYWLLYNMLLAAVLGFYFLLWEMCRIQKLINDQMTLILARSGQRNRLKKMQHCLRLYSKLLLLCDQFNSQLGHVAIWVLACKSWCQITFGYEIFQMVAAPKSIDLTMSMRVFVIFTYIFDAMNLFLGTDISELFSTFRADSQRILRETSRLDRLLSMFALKLALHPKRVVLLNVFTFDRKLTLTLLAKSTLYTICCLQNDYNKLKA

SEQ ID NO: 36 - protein, 395 aa, Drosophila melanogaster
MLLKFMYIYGIGCGLMPAPLKKGQFLLGYKQRWYLIYTACLHGGLLTVLPFTFPHYMYDDSYMSSNPVLKWTFNLTNITRIMAMFSGVLLMWFRRKRILNLGENLILHCLKCKTLDNRSKKYSKLRKRVRNVLFQMLLVANLSILLGALILFRIHSVQRISKTAMIVAHITQFIYVVFMMTGICVILLVLHWQSERLQIALKDLCSFLNHEERNSLTLSENKANRSLGKLAKLFKLFAENQRLVREVFRTFDLPIALLLLKMFVTNVNLVYHGVQFGNDTIETSSYTRIVGQWVVISHYWSAVLLMNVVDDVTRRSDLKMGDLLREFSHLELVKRDFHLQLELFSDHLRCHPSTYKVCGLFIFNKQTSLAYFFYVLVQVLVLVQFDLKNKVEKRN

SEQ ID NO: 37 - protein, 408 aa, Drosophila melanogaster
MLHPKLGRVMNVVYYHSVVFALMSTTLRIRSCRKCLRLEKVSRTYTIYSFFVGIFLFLNLYFMVPRIMEDGYMKYNIVLQWNFFVMLFLRAIAVVSCYGTLWLKRHKIIQLYKYSLIYWKRFGHITRAIVDKKELLDLQESLARIMIRKIILLYSAFLCSTVLQYQLLSVINPQIFLAFCARLTHFLHFLCVKMGFFGVLVLLNHQFLVIHLAINALHGRKARKKWKALRSVAAMHLKTLRLARRIFDMFDIANATVFINMFMTAINILYHAVQYSNSSIKSNGWGILFGNGLIVFNFWGTMALMEMLDSVVTSCNNTGQQLRQLSDLPKVGPKMQRELDYFTMQLRQNRLVYKICGIVELDKPACLSYIGSILSNVIILMQFDLRRQRQPINDRQYLIHLMKNKTKV

SEQ ID NO: 38 - protein, 412 aa, Drosophila melanogaster
MNQYFLLHTYFQVSRLIGLCNLHYDSSNHRFILNHVPTVVYCVILNVVYLLVLPFALFVLTGNIYHCPDAGMFGVVYNVVALTKLLTMLFLMSSVWIQRRRLYKLGNDLMKMLHKFRFNLGNDCRNRCLCKGLLTSSRFVLLTQQLLTRDSVVNCESNSSLRQAMVPYQSAAIVYALIMILLMSYVDMTVYMVEVAGNWLLVNMTQGVREMVQDLEVLPERNGIPREMGLMQILAAWRKLWRRCRRLDALLKQFVDIFQWQVLFNLLTTYIFSIAVLFRLWIYLEFDKNFHLWKGILYAIIFLTHHVEIVMQFSIFEINRCKWLGLLEDVGNLWDINYSGRQCIKSSGTILSRKLEFSLLYMNRKLQLNPKRVRRLHIVGLFDISNLTVHNMTRSIITNVLVLCQIAYKKYG

SEQ ID NO: 39 - protein, 390 aa, Drosophila melanogaster
MADLLKLCLRIAYAYGRLTGVINFKIDLKTGQALVTRGATLISVSTHLLIFALLLYQTMRKSVVNVMWKYANSLHEYVFLVIAGFRVVCVFLELVSRWSQRRTFVRLFNSFRRLYQRNPDIIQYCRRSIVSKFFCVTMTETLHIIVTLAMMRNRLSIALALRIWAVLSLTAIINVIITQYYVATACVRGRYALLNKDLQAIVTESQSLVPNGGGVFVTKCCYLADRLERIAKSQSDLQELVENLSTAYEGEVVCLVITYYLNMLGTSYLLFSISKYGNFGNNLLVIITLCGIVYFVFYVVDCWINAFNVFYLLDAHDKMVKLLNKRTLFQPGLDHRLEMVFENFALNLVRNPLKLHMYGLFEFGRGTSFAVFNSLLTHSLLLIQYDVQNF

SEQ ID NO: 40 - protein, 394 aa, Drosophila melanogaster
MVDLVKTILLIAYWYGLAVGVSNFEVDWLTGEAIATRRTTIYAAVHNASLITLLILFNLGNNSLKSEFISARYLHEYFFMLMTAVRISAVLLSLITRWYQRSRFIRIWNQILALVRDRPQVVRGRWYRRSIILKFVFCVLSDSLHTISDVSAQRKRITADLIVKLSLLATLTTIFNMIVCQYYLAMVQVIGLYKILLQDLRCLVRQAECICSIRNRRGGVYSIQCCSLADQLDLIAERHYFLKDRLDEMSDLFQIQSLSMSLVYFFSTMGSIYFSVCSILYSSTGFGSTYWGLLLIVLSTASFYMDNWLSVNIGFHIRDQQDELFRVLADRTLFYRELDNRLEAAFENFQLQLASNRHEFYVMGLFKMERGRLIAMLSSVITHTMVLVQWEIQN

SEQ ID NO: 41 - protein, 405 aa, Drosophila melanogaster
MRSSATKGAKLKNSPRERLSSFNPQYAERYKELYRTLFWLLLISVLANTAPITILPGCPNRFYRLVHLSWMILWYGLFVLGSYWEFVLVTTQRVSLDRYLNAIESAIYVVHIFSIMLLTWQCRNWAPKLMTNIVTSDLNRAYTIDCNRTKRFIRLQLFLVGIFACLAIFFNIWTHKFVVYRSILSINSYVMPNIISSISFAQYYLLLQGIAWRQRRLTEGLERELTHLHSPRISEVQKIRMHHANLIDFTKAVNRTFQYSILLLFVGCFLNFNLVLFLVYQGIENPSMADFTKWVCMLLWLAMHVGKVCSILHFNQSIQNEHSTCLTLLSRVSYARKDIQDTITHFIIQMRTNVRQHVVCGVINLDLKFLTTLLVASADFFIFLLQYDVTYEALSKSVQGNVTRY

SEQ ID NO: 42 - protein, 399 aa, Drosophila melanogaster
MDSSYWENLLLTINRFLGVYPSGRVGVLRWLHTLWSLFLLMYIWTGSIVKCLEFTVEIPTIEKLLYLMEFPGNMATIAILVYYAVLNRPLAHGAELQIERIITGLKGKAKRLVYKRHGQRTLHLMATTLVFHGLCVLVDVVNYDFEFWTTWSSNSVYNLPGLMMSLGVLQYAQPVHFLWLVMDQMRMCLKELKLLQRPPQGSTKLDACYESAFAVLVDAGGGSALMIEEMRYTCNLIEQVHSQFLLRFGLYLVLNLLNSLVSICVELYLIFNFFETPLWEESVLLVYRLLWLAMHGGRIWFILSVNEQILEQKCNLCQLLNELEVCSSRLQRTINRFLLQLQRSIDQPLEACGIVTLDTRSLGGFIGVLMAIVIFLIQIGLGNKSLMGVALNRSNWVYV

SEQ ID NO: 43 - protein, 392 aa, Drosophila melanogaster
MKIYQDIYPISKPSQIFAILPFYSGDVDDGFRFGGLGRWYGRLVALIILIGSLTLGEDVLFASKEYRLVASAQGDTEEINRTIETLLCIISYTMVVLSSVQNASRHFRTLHDIAKIDEYLLANGFRETYSCRNLTILVTSAAGGVLAVAFYYIHYRSGIGAKRQIILLLIYFLQLLYSTLLALYLRTLMMNLAQRIGFLNQKLDTFNLQDCGHMENWRELSNLIEVLCKFRYITENINCVAGVSLLFYFGFSFYTVTNQSYLAFATLTAGSLSSKTEVADTIGLSCIWVLAETITMIVICSACDGLASEVNGTAQILARIYGKSKQFQNLIDKFLTKSIKQDLQFTAYGFFSIDNSTLFKIFSAVTTYLVILIQFKQLEDSKNLSRSYQLVM

SEQ ID NO: 44 - protein, 424 aa, Drosophila melanogaster
MPRWLQLPGMSALGILYSLTRVFGLMATANWSPRGIKRVRQSLYLRIHGCVMLIFVGCFSPFAFWCIFQRMAFLRQNRILLMIGFNRYVLLLVCAFMTLWIHCFKQAEIIGCLNRLLKCRRRLRRLMHTRKLKDSMDCLATKGHLLEVVVLLSSYLLSMAQPIQILKDDPEVRRNFMYACSLVFVSVCQAILQLSLGMYTMAILFLGHLVRHSNLLLAKILADAEHIFESSQKAGFWPNRQELYKGQQKWLALELWRLLHVHHQLLKLHRSICSLCAVQAVCFLGFVPLECTIHLFFTYFMKYSKFILRKYGRSFPLNYFAIAFLVGLFTNLLLVILPTYYSERRFNCTREIIKGGGLAFPSRITVKQLRHTMHFYGLYLKNVEHVFAVSACGLFKLNNAILFCIVGAILEYLMILIQFDKVLN

SEQ ID NO: 45 - protein, 92 aa, Drosophila melanogaster
CQLLNGYRTEHAGGNYLLASDFDRRLKVFLQWKTSDSAESASGRLGSQYTFVGHKKKQTGLTIKLAENGFCCWVLLLRYFSVLIKIVKYKIP

SEQ ID NO: 46 - protein, 416 aa, Drosophila melanogaster
MAVLYFFREPETVFDCAAFICILQFLMGCNGFGIRRSTFRISWASRIYSMSVAIAAFCCLFGSLSVLLAEEDIRERLAKADNLVLSISALELLMSTLVFGVTVISLQVFARRHLGIYQRLAALDARLMSDFGANLNYRKMLRKNIAVLGIVTTIYLMAINSAAVQVASGHRALFLLFALCYTIVTGGPHFTGYVHMTLAEMLGIRFRLLQQLLQPEFLNWRFPQLHVQELRIRQVVSMIQELHYLIQEINRVYALSLWAAMAHDLAMSTSELYILFGQSVGIGQQNEEENGSCYRMLGYLALVMIPPLYKLLIAPFYCDRTIYEARRCLRLVEKLDDWFPQKSSLRPLVESLMSWRIQAKIQFTSGLDVVLSRKVIGLFTSILVNYLLILIQFAMTQKMGEQIEQQKIALQEWIGF

SEQ ID NO: 47 - protein, 339 aa, Drosophila melanogaster
MRVHQRQSAVIIQMGHPPFMSLKGGKSGFGSIVWPSAMREVNLLNRFTRQFLFLIVLVTQICGVATFVYNSKAQCFRQSGFLRFYSSLVLIFLALFLIVTTSKMFHNLQAVWPYVVGSVIILVVRIHGLLESAEIVELLNQMLRIMRQVNLMARHPNLFRLKHLLLLLLALQNLLRSLNTIVGISNHSAEAYDSFLNSVILLIILAVLLSFLLQITINICLFVVLIATYSELHHCTRRISNDMDKLRLHSVHESGQFMVLVKQLQGITEKLIRLRQNVFHITVRIIRHFRFHWLCAIIYGLLPFFSLTAKDQNGFNFLIISALNIIFQWTIFAILSRES

SEQ ID NO: 48 - protein, 417 aa, Drosophila melanogaster
MTGKRAESWSRLLLLWLYRCARGLLVLSSSLDRDKLQLKATKQGSRNRFLHILWRCIVVMIYAGLWPMLTSAVIGKRLESYADVLALAQSMSVSILAVISFVIQARGENQFREVLNRYLALYQRICLTTRLRHLFPTKFVVFFLLKLFFTLCGCFHEIIPLFENSHFDDISQMVGTGFGIYMWLGTLCVLDACFLGFLVSGILYEHMANNIIAMLKRMEPIESQDERYRMTKYRRMQLLCDFADELDECAAIYSELYHVTNSFRRILQWQILFYIYLNFINICLMLYQYILHFLNDDEVVFVSIVMAFVKLANLVLLMMCADYTVRQSEVPKKLPLDIVCSDMDERWDKSVSLLLFETFLGQLQTQRLEIKVLGFFHLNNEFILLILSAIISYLFILIQFGITGGFEASEDIKNFAD

SEQ ID NO: 49 - protein, 299 aa, Drosophila melanogaster
MQFWFGEELINLVNRFLQLFRRMQSLTNSPKNRFGDRAEFLLMFSKVFSLLFVFMAFRLMLSPWFLLTLVCDLYTSVGTGMITHLCFVGYLSIGVLYRDLNNYVDCQLRAQLRSLNGENNSFRNNPQPTRQAISNLDKCLYLYDEIHQVSRSFQQLFDLPLFLSLAQSLLAMSMVSYHAILRRQYSFNLWGLVIKLLIDVVLLTMSVHSAVNGSRLIRRLSFENFYVTDSQSYHQKVSPGAIILRIKYNTFPILQLELFLGRLQHQELRVFPLGLFEVSNELTLFFLSAMVTYLVFLVQ

SEQ ID NO: 50 - protein, 407 aa, Drosophila melanogaster
MIERLKKVSLPALSAFILFCSCHYGRILGVICFDIGQRTSDDSLVVRNRHQFKWFCLSCRLISVTAVCCFCAPYVADIEDPYERLLQCFRLSASLICGICIIVVQVCYEKELLRMIISFLRLFRRVRRLSSLKRIGFGGKREFFLLLFKFICLVYELYSEICQLWHLPDSLSLFATLCEIFLEIGSLMIIHIGFVGYLSVAALYSEVNSFARIELRRQLRSLERPVGGPVGRKQLRIVEYRVDECISVYDEIERVGRTFHRLLELPVLIILLGKIFATTILSYEVIIRPELYARKIGMWGLVVKSFADVILLTLAVHEAVSSSRMMRRLSLENFPITDHKAWHMKVSDLMVFLIKCIFFSRLQWEMFLSRLNFFEFRVRPLGLFEVSNEVILLFLSSMITYFTYVVQ

SEQ ID NO: 51 - protein, 363 aa, Drosophila melanogaster
MSFYARFLSLVCFRLRKQKDNNVWLEEIWSNRSRWKWISVTLRIVPLCIYAFTYAEWISNRMLITEKFLHSCSLVVSIPCYLSIIHLKICHGPEVTKLVNQYLHIFRLGTLDIRRRSQFGGGRELFLLILSVCCQIHEYVFILVIASRLCGFQHIIWWVSYTYVFIICNSIMCFGFIWHLSLGVLYAELNDNLRFESGFQTAFLRKQQRIRVQKSMALFKEISSVVTSLQDIFNVHLFLSALLTLLQVLVVWYKMIIDLGFSDFRIWSFSLKNLIQTLLPVLAIQEAANQFKQTRERALDIFLVGKSKHWMKSVSKLINQGILQLIGLFNVSNELFLIIVSAMFCYLVFVTQCVIVYRRRYVI

SEQ ID NO: 52 - protein, 404 aa, Drosophila melanogaster
MDFTSDYAHRRMVKFLTIILIGFMTVFGLLANRYRAGRRERFRFSKANLAFASLWAIAFSLVYGRQIYKEYQEGQINLKDATTLYSYMNITVAVINYVSQMIISDHVAKVLSKVPFFDTLKEFRLDSRSLYISIVLALVKTVAFPLTIEVAFILQQRRQHPEMSLIWTLYRLFPLIISNFLNNCYFGAMVVVKEILYALNRRLEAQLQEVNLLQRKDQLKLYTKYYRMQRFCALADELDQLAYRYRLIYVHSGKYLTPMSLSMILSLICHLLGITVGFYSLYYAIADTLIMGKPYDGLGSLINLVFLSISLAEITLLTHLCNHLLVATRRSAVILQEMNLQHADSRYRQAVHGFTLLVTVTKYQIKPLGLYELDMRLISNVFSAVASFLLILVQADLSQRFKMQ

SEQ ID NO: 53 - protein, 352 aa, Drosophila melanogaster
MRFLRRQTRRLRSIWQRSLPVRFRRGKLHTQLVTICLYATVFLNILYGVYLGRFSFRRKKFVFSKGLTIYSLFVATFFALFYIWNIYNEISTGQINLRDTIGIYCYMNVCVCLFNYVTQWEKTLQIIRFQNSVPLFKVLDSLDISAMIVWRAFIYGLLKIVFCPLITYITLILYHRRSISESQWTSVTTTKTMLPLIVSNQINNCFFGGLVLANLIFAAVNRKLHGIVKEANMLQSPVQMNLHKPYYRMRRFCELADLLDELARKYGFTASRSKNYLRFTDWSMVLSMLMNLLGITMGCYNQYLAIADHYINEEPFDLFLAIVLVVFLAVPFLELVMVARISNQTLVEVIVI

SEQ ID NO: 54 - protein, 160 aa, Drosophila melanogaster
IERFVCAQLVHEAYKQFASNGFRFLDALGCYEHSALGRARPLSRRGYAIKVSDHPATPPHYHMPPPKQPPSHLAVQHATLTSGLRQLSFSCVNCNCSRCCWSLPMHFRYIFNASLCNCQRQGYTLSCRRHCTATKNISFSFCHISFVFLLKYDPKNPQLR

SEQ ID NO: 55 - protein, 405 aa, Drosophila melanogaster
MFDWVGLLLKVLYYYGQIIGLINFEIDWQRGRVVAAQRGILFAIAINVLICMVLLLQISKKFNLDVYFGRANQLHQYVIIVMVSLRMASLNRWRQRAQLMRLVECVLRLFLKKPHVKQMSRWAILVKFSVGVVSNFLQMAISMESLDRLGFNEFVGMASDFWMSAIINMAISQHYLVILFVRAYYHLLKTEVRQAIHESQMLSEIYPRRAAFMTKCCYLADRIDNIAKLQNQLQSIVTQLNQVFGIQGIMVYGGYYIFSVATTYITYSLAINGIEELHLSVRAAALVFSWFLFYYTSAILNLFVMLKLFDDHKEMERILEERTLFTSALDVRLEQSVSFYPTITELKYRDLVLSQFESIQLQLIRNPLKIEVLDIFTITRSSSAAMIGSIITNSIFLIQYDMEYF

SEQ ID NO: 56 - protein, 365 aa, Drosophila melanogaster
MWLLRRSVGKSGNRPHDVYTCYRLTIFMALCLGIVPYYVSISSEGRGKLTSSYIGYINIIIRMAIYMVNSFYGAVNRDTLMSNFFLTDISNVIDALQKINGMLGIFAILLISLLNRKELLKLLATFDRLETEAFPRVLKNLAHQWDTRSLKAVNQKQRSLQCLDSFSMYTIVTKDPAEIIQESMEIHHLICEAAATANKYFTYQLLTIISIAFLIIVFDAYYVLETLLGKSKRESKFKTVEFVTFFSCQMILYLIAIISIVEGSNRAIKKSEKTGGIVHSLLNKTKSAEVKEKLQQFSMQLMHLKINFTAAGLFNIDRTLYFTISGALTTYLIILLQFTSNSPNNGYGNGSSCCETFNNMTNHTL

SEQ ID NO: 57 - protein, 450 aa, Drosophila melanogaster
MKGPNLNFRKTPSKDNGVKQVESLARPETPPPKFVEDSNLEFNVLASEKLPNYTNLDLFHRAVFPFMFLAQCVAIMPLVGIRESNPRRVRFAYKSIPMFVTLIFMIATSILFLSMFTHLLKIGITAKNFVGLVFFGCVLSAYVVFIRLAKKWPAVVRIWTRTEIPFTKPPYEIPKRNLSRRVQLAALAIIGLSLGEHALYQVSAILSYTRRIQMCANITTVPSFNNYMQTNYDYVFQLLPYSPIIAVLILATCTFVWNYMDLFIMMISKGLSYRFEQITTRIRKLEHEEVCESVFIQIREHYVKMCELLEFVDSAMSSLILLSCVNNLYFVCYQLLNVFNKLRWPINYIYFWYSLLYLIGRTAFVFLTAADINEESKRGLGVLRRVSSRSWCVEVERLIFQMTTQTVALSGKKFYFLTRRLLFGMAGTIVTYELVLLQFDEPNRRKGLQP

SEQ ID NO: 58 - protein, 28 aa, Drosophila melanogaster
IYILSLYIFFQFISNVSLIVVLKLFRDI

SEQ ID NO: 59 - protein, 444 aa, Drosophila melanogaster
MRQLKGRNRCNRAVRHLKVQGKMWLKNLKSGLEQIRESQVRGTRKNFLHDGSFHEAVAPVLAVAQCFCLMPVCGISAPTYRGLSFNRRSWRFWYSSLYLCSTSVDLAFSIRRVAHSVLDVRSVEPIVFHVSILIASWQFLNLAQLWPGLMRHWAAVERRLPGYTCCLQRARPARRLKLVAFVLLVVSLMEHLLSIISVVYYDFCPRRSDPVESYLLGASAQLFEVFPYSNWLAWLGKIQNVLLTFGWSYMDIFLMMLGMGLSEMLARLNRSLEQQVRQPMPEAYWTWSRTLYRSIVELIREVDDAVSGIMLISFGSNLYFICLQLLKSINTMPSSAHAVYFYFSLLFLLSRSTAVLLFVSAINDQAREPLRLLRLVPLKGYHPEVFRFAAELASDQVALTGLKFFNVTRKLFLAMAGTVATYELVLIQFHEDKKTWDCSPFNLD

SEQ ID NO: 60 - protein, 25 aa, Artificial sequence
  motif
GXFXXXXXXXXXXXXXXTYLXLXQF

SEQ ID NO: 61 - protein, 33 aa, Drosophila melanogaster
FRFQLCGLFSINHNMGFQMIITSFLYLVYLLQF

SEQ ID NO: 62 - protein, 33 aa, Drosophila melanogaster
LQLWSCGLFQANRSMWFAMISSVLYYILVLLQF

SEQ ID NO: 63 - protein, 33 aa, Drosophila melanogaster
STYKVCGLFIFNKQTSLAYFFYVLVQVLVLVQF

SEQ ID NO: 64 - protein, 33 aa, Drosophila melanogaster
HEFYVMGLFKMERGRLIAMLSSVITHTMVLVQW

SEQ ID NO: 65 - protein, 33 aa, Drosophila melanogaster
LEIKVLGFFHLNNEFILLILSAIISYLFILIQF

SEQ ID NO: 66 - protein, 33 aa, Drosophila melanogaster
PIMNLDGYANINRELITTNISFMATYLVVLLQF

SEQ ID NO: 67 - protein, 33 aa, Drosophila melanogaster
STINCGGFFDVNRTLFKGLLTTMVTYLVVLLQF

SEQ ID NO: 68 - protein, 33 aa, Drosophila melanogaster
LAINAEGFMSTDNSLLMSILAAKVTYLIVLMQF

SEQ ID NO: 69 - protein, 33 aa, Drosophila melanogaster
INFTAAGLFNIDRTLYFTISGALTTYLIILLQF

SEQ ID NO: 70 - protein, 33 aa, Drosophila melanogaster
LHFSAAGFFNVDCTLLYTIVGATTTYLIILIQF

SEQ ID NO: 71 - protein, 33 aa, Drosophila melanogaster
ADFSACGLCRVNRTILTSFASAIATYLVILIQF

SEQ ID NO: 72 - protein, 32 aa, Drosophila melanogaster
FMTCAASFMSNRVTIQVCLKAIFTYMVILVQF

SEQ ID NO: 73 - protein, 33 aa, Drosophila melanogaster
VALTGMKFFHLTRKLVLSVAGTIVTYELVLIQF

SEQ ID NO: 74 - protein, 33 aa, Drosophila melanogaster
VALTGLKFFNVTRKLFLAMAGTVATYELVLIQF

SEQ ID NO: 75 - protein, 33 aa, Drosophila melanogaster
MSISGAKFFTVSLDLFASVLGAVVTYFMVLVQL

SEQ ID NO: 76 - protein, 33 aa, Drosophila melanogaster
VELNAMGYLSISLDTFKQLMSVSYRVITMLMQM

SEQ ID NO: 77 - protein, 33 aa, Drosophila melanogaster
ITLTAGGVFPISMQTNLAMVKLAFSVVTVIKQF

SEQ ID NO: 78 - protein, 33 aa, Drosophila melanogaster
IILTAGGVFPISMQTNLNMVKLAFTVVTIVKQF

SEQ ID NO: 79 - protein, 33 aa, Drosophila melanogaster
IVFIAGGIFQISMSSNISVAKFAFSVITITKQM

SEQ ID NO: 80 - protein, 33 aa, Drosophila melanogaster
IIFIAGGIFPISMNSNITVAKFAFSIITIVRQM

SEQ ID NO: 81 - protein, 33 aa, Drosophila melanogaster
IQFTAGSTFPISVQSNIAVAKFAFTIITIVNQM

SEQ ID NO: 82 - protein, 33 aa, Drosophila melanogaster
IAFTAGSIFPISTGSNIKVAKLAFSVVTFVNQL

SEQ ID NO: 83 - protein, 33 aa, Drosophila melanogaster
ILFTAGGIFPICLNTNIKMAKFAFSVVTIVNEM

SEQ ID NO: 84 - protein, 33 aa, Drosophila melanogaster
ITLTAMKLFPINLATYFSIAKFSFSLYTLIKGM

SEQ ID NO: 85 - protein, 33 aa, Drosophila melanogaster
IRIDCLGLTILDCSLLTRMACSVGTYMIYSIQF

SEQ ID NO: 86 - protein, 33 aa, Drosophila melanogaster
FQFNGVGLFALDYTFIFSTVSAATSYLIVLLQF

SEQ ID NO: 87 - protein, 33 aa, Drosophila melanogaster
VDFSACGFFTLDMETLYGVSGGITSYLIILIQF

SEQ ID NO: 88 - protein, 32 aa, Drosophila melanogaster
PPMLCGLLHLDRRLVYLIAVTAFSYFITLVQF

SEQ ID NO: 89 - protein, 33 aa, Drosophila melanogaster
YQIKPLGLYELDMRLISNVFSAVASFLLILVQA

SEQ ID NO: 90 - protein, 33 aa, Drosophila melanogaster
IQFTSGLDVVLSRKVIGLFTSILVNYLLILIQF

SEQ ID NO: 91 - protein, 33 aa, Drosophila melanogaster
QPLEACGIVTLDTRSLGGFIGVLMAIVIFLIQI

SEQ ID NO: 92 - protein, 31 aa, Drosophila melanogaster
FRITGYFFEANMEAFSSIVRTAMSYITMLRS

SEQ ID NO: 93 - protein, 31 aa, Drosophila melanogaster
CQMKGYFFEASMATFSTIVRSAVSYIMMLRS

SEQ ID NO: 94 - protein, 31 aa, Drosophila melanogaster
MKMRALLVDLNLRTFIDIGRGAYSYFNLLRS

SEQ ID NO: 95 - protein, 31 aa, Drosophila melanogaster
AKIFGFMFVVDLPLLLWVIRTAGSFLAMLRT

SEQ ID NO: 96 - protein, 32 aa, Drosophila melanogaster
LASLVGGTYPMNLKMLQSLLNAIYSFFTLLRR

SEQ ID NO: 97 - protein, 32 aa, Drosophila melanogaster
NEIRVGNVYPMTLAMFQSLLNASYSYFTMLRG

SEQ ID NO: 98 - protein, 32 aa, Drosophila melanogaster
AAILLGNIRPITLELFQNLLNTTYTFFTVLKR

SEQ ID NO: 99 - protein, 32 aa, Drosophila melanogaster
QLLLAGNLVPIHLSTYVACWKGAYSFFTLMAD

SEQ ID NO: 100 - protein, 32 aa, Drosophila melanogaster
SLIYAGNYIALSLETFEQVMRFTYSVFTLLLR

SEQ ID NO: 101 - protein, 32 aa, Drosophila melanogaster
VNIKAGGIVGIDMSAFFATVRMAYSFYTLALS

SEQ ID NO: 102 - protein, 32 aa, Drosophila melanogaster
VQIKAGGMIGIGMNAFFATVRLAYSFFTLAMS

SEQ ID NO: 103 - protein, 32 aa, Drosophila melanogaster
WIIKAGGLIELNLNAFFATLKMAYSLFAVVHR

SEQ ID NO: 104 - protein, 32 aa, Drosophila melanogaster
STAVAGGMMRIHLDTFFSTLKGAYSLFTIIIR

SEQ ID NO: 105 - protein, 32 aa, Drosophila melanogaster
VTIRAGNSFAVGLPIFVKTINNAYSFLALLLN

SEQ ID NO: 106 - protein, 32 aa, Drosophila melanogaster
VKVRAGVFFEIGLPIFVKTINNAYSFFALLLK

SEQ ID NO: 107 - protein, 33 aa, Drosophila melanogaster
VTLKAGGFFHIGLPLFTKVVFSTLENPCISYLY

SEQ ID NO: 108 - protein, 32 aa, Drosophila melanogaster
VSMAVPFFSPSLATFAAILQTSGSIIALVKSF

SEQ ID NO: 109 - protein, 33 aa, Drosophila melanogaster
LMYVAEPFLPFTLGTYMLVLKNCYRLLALMQES

SEQ ID NO: 110 - protein, 33 aa, Drosophila melanogaster
FFITGLNYFRVSLTAVLKIIQGAFSYFTFLNSM

SEQ ID NO: 111 - protein, 33 aa, Drosophila melanogaster
QQLGAFGLIQVNMVHFTEIMQLAYRLFTFLKSH

SEQ ID NO: 112 - protein, 33 aa, Drosophila melanogaster
VHVTAGKFYVMDVNRLRSVITQAFSFLTLLQKL

SEQ ID NO: 113 - protein, 33 aa, Drosophila melanogaster
HNIQILGVMSLSVRTALQIVKLIYSVSMMMMNR

SEQ ID NO: 114 - protein, 33 aa, Drosophila melanogaster
KRVVLLNVFTFDRKLTLTLLAKSTLYTICCLQN

SEQ ID NO: 115 - protein, 33 aa, Drosophila melanogaster
RQHVVCGVINLDLKFLTTLLVASADFFIFLLQY

SEQ ID NO: 116 - protein, 28 aa, Drosophila melanogaster
TVLGAYFFELGRPLLVWVSIFLFIVLLF

(19) United States
(12) Patent Application Publication
(10) Pub. No.: US 2009/0093022 A1
(43) Pub. Date: Apr. 9, 2009

(54) Chemosensory gene family encoding gustatory and odorant receptors and uses thereof

(75) Inventors: Richard Axel, New York, NY (US); Kristin Scott, New York, NY (US)

Correspondence Address: John P. White;Cooper & Dunham LLP, 1185 Avenue of the Americas, New York, NY, 10036, US

(73) Assignee: The Trustees of Columbia University in The City of New York

(21) Appl. No.: 12/287,781

(22) Filed: Oct. 14, 2008

Related U.S. Application Data
(63) Continuation of application No. 10/081,816, filed on Feb. 22, 2002, now abandoned.
(60) Provisional application No. 60/271,319, filed on Feb. 23, 2001.

(51) Int. Cl. C12P 21/00; C07H 21/04; C12N 15/63

(52) U.S. Cl. 435/69.1; 536/23.5; 435/320.1

(57) ABSTRACT

This invention provides an isolated nucleic acid encoding an insect gustatory or odorant receptor. This invention provides a nucleic acid of at least 12 nucleotides capable of specifically hybridizing with a nucleic acid encoding an insect gustatory or odorant receptor. This invention also provides a purified, insect gustatory or odorant receptor. This invention provides an antibody capable of specifically binding to an insect gustatory or odorant receptor. This invention provides a method of identifying a compound capable of specifically binding to, activating, or inhibiting the activity of an insect gustatory or odorant receptor. This invention also provides methods of controlling insect populations.

This application claims the benefit of U.S. Provisional Application No. 60/271,319, filed Feb. 23, 2001, the contents of which are hereby incorporated by reference.

The invention disclosed herein was made with Government support under grant numbers NS 29832-09 from the National Institutes of Health and 2POICA23767-22 from the National Cancer Institute. Accordingly, the U.S. Government has certain rights in this invention.

### BACKGROUND OF THE INVENTION

Throughout this application, various publications are referenced in parentheses. Full citations for these references may be found at the end of the specification immediately preceding the claims. The disclosures of these publications in their entireties are hereby incorporated by reference into this application to more fully describe the state of the art to which this invention pertains.

All animals have specialized mechanisms to recognize and respond to chemosensory information in the environment. Olfactory neurons recognize volatile cues that afford the organism the ability to detect food, predators and mates. In contrast, gustatory neurons sense soluble chemical cues that elicit feeding behaviors. In insects, taste neurons also initiate innate sexual and reproductive responses. In Drosophila, for example, sweet compounds are recognized by chemosensory hairs on the proboscis and legs that activate proboscis extension and feeding (Dethier, 1976). Sexually dimorphic chemosensory bristles on the foreleg of males recognize cues from receptive females that are thought to elicit the embrace of mating (Tompkins et al., 1983; Possidente and Murphey, 1989). Females have yet a third set of specialized bristles on their genitalia that may cause oviposition in response to nutrients (Rice, 1977; Taylor, 1989). In this manner, gravid females will preferentially deposit their eggs on a rich environment that enhances survival of their offspring. These robust and innate gustatory responses provide the opportunity to understand how chemosensory information is recognized in the periphery and ultimately translated into specific behaviors.

Taste in Drosophila is mediated by sensory bristles that reside on the proboscis, legs, wing, and genitalia (Stocker, 1994; Singh, 1997). Most chemosensory bristles are innervated by four bipolar gustatory neurons and a single mechanoreceptor cell (Falk et al., 1976). The dendrites of gustatory neurons extend into the shaft of the bristle and are the site of taste recognition that translates the binding of tastants into alterations in membrane potential. The sensory axons from the proboscis project to the brain where they synapse on projection neurons within the subesophageal ganglion (SOG), the first relay station for gustatory information in the fly brain (Stocker and Schorderet, 1981; Nayak and Singh, 1983; Shanbhag and Singh, 1992; Rajashekhar and Singh, 1994). Sensory axons from taste neurons at other sites along the body project locally to peripheral ganglia (Power, 1948). Drosophila larvae, whose predominant activity is eating, sense their chemical environment with gustatory neurons that reside in chemosensory organs on the head and are also distributed along the body surface (Stocker, 1994) The pattern of projection of functionally distinct classes of taste cells and therefore the nature of the representation of gustatory information in the Drosophila brain remains unknown.

The identification of the genes encoding taste receptors and the analysis of the patterns of receptor expression may provide insight into the logic of taste discrimination in the fly. In Drosophila, the recognition of odorants is thought to be accomplished by about 70 seven-transmembrane domain proteins encoded by the Drosophila odorant receptor (DOR) gene family (Clyne et al., 1999; Gao and Chess, 1999; Vosshall et al., 1999; Vosshall et al., 2000). Recently, a large family of putative G protein-coupled receptors was identified by searching the Drosophila genome with an algorithm designed to detect seven-transmembrane domain proteins (Clyne et al., 2000). These genes were suggested to encode gustatory receptors (GRs) because members of this gene family were detected in the proboscis by RT-PCR experiments.

The present application characterizes and extends the family of putative G protein-coupled receptors originally identified by Clyne et al. (2000) and provides evidence that they encode both olfactory and gustatory receptors. In situ hybridization, along with transgene experiments, reveals that some receptors are expressed in topographically restricted sets of neurons in the proboscis, whereas other members are expressed in spatially fixed olfactory neurons in the antenna. Members of this gene family are also expressed in chemosensory bristles on the leg and in larval chemosensory organs. Finally, the projections of different subsets of larval chemosensory neurons were traced to the subesophageal ganglion and the antennal lobe. These data provide insight into the diversity of chemosensory recognition in the periphery and afford an initial view of the representation of gustatory information in the fly brain.

### SUMMARY OF THE INVENTION

This invention provides an isolated nucleic acid encoding an insect gustatory receptor protein, wherein the receptor protein comprises seven transmembrane domains and a C-terminal domain, and the C-terminal domain comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

The invention provides an isolated nucleic acid encoding an insect odorant receptor protein, wherein the receptor protein comprises seven transmembrane domains and a C-terminal domain, and the C-terminal domain comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

The invention provides an isolated nucleic acid encoding an insect gustatory receptor protein, wherein the nucleic acid molecule encodes a protein selected from the group consisting of:
(a) an insect receptor protein comprising consecutive amino acids having a sequence identical to that set forth for Gr2B1 in SEQ ID NO: 1,
(b) an insect receptor protein comprising consecutive amino acids having a sequence identical to that set forth for Gr8D1 in SEQ ID NO: 2,
(c) an insect receptor protein comprising consecutive amino acids having a sequence identical to that set forth for Gr10B1 in SEQ ID NO: 3,
(d) an insect receptor protein comprising consecutive amino acids having a sequence identical to that set forth for Gr10B2 in SEQ ID NO: 4,
(e) an insect receptor protein comprising consecutive amino acids having a sequence identical to that set forth for Gr28A2 in SEQ ID NO: 5,
(f) an insect receptor protein comprising consecutive amino acids having a sequence identical to that set forth for Gr28A4 in SEQ ID NO: 6,
(g) an insect receptor protein comprising consecutive amino acids having a sequence identical to that set forth for Gr33C1 in SEQ ID NO: 7,
(h) an insect receptor protein comprising consecutive amino acids having a sequence identical to that set forth for Gr36B2 in SEQ ID NO: 8,
(i) an insect receptor protein comprising consecutive amino acids having a sequence identical to that set forth for Gr36B3 in SEQ ID NO: 9,
(j) an insect receptor protein comprising consecutive amino acids having a sequence identical to that set forth for Gr59C1 in SEQ ID NO: 10,
(k) an insect receptor protein comprising consecutive amino acids having a sequence identical to that set forth for Gr61D1 in SEQ ID NO: 11,
(l) an insect receptor protein comprising consecutive amino acids having a sequence identical to that set forth for Gr63F1 in SEQ ID NO: 12,
(m) an insect receptor protein comprising consecutive amino acids having a sequence identical to that set forth for Gr64A2 in SEQ ID NO: 13,
(n) an insect receptor protein comprising consecutive amino acids having a sequence identical to that set forth for GR64A3 in SEQ ID NO: 14,
(o) an insect receptor protein comprising consecutive amino acids having a sequence identical to that set forth for Gr66C1 in SEQ ID NO: 15,
(p) an insect receptor protein comprising consecutive amino acids having a sequence identical to that set forth for Gr92D1 in SEQ ID NO: 16,
(q) an insect receptor protein comprising consecutive amino acids having a sequence identical to that set forth for Gr98A1 in SEQ ID NO: 17,
(r) an insect receptor protein comprising consecutive amino acids having a sequence identical to that set forth for Gr98A2 in SEQ ID NO: 18,
(s) an insect receptor protein comprising consecutive amino acids having a sequence identical to that set forth for Gr2940.1 in SEQ ID NO: 19,
(t) an insect receptor protein comprising consecutive amino acids having a sequence identical to that set forth for Gr2940.2 in SEQ ID NO: 20,
(u) an insect receptor protein comprising consecutive amino acids having a sequence identical to that set forth for Gr2940.3 in SEQ ID NO: 21,
(v) an insect receptor protein comprising consecutive amino acids having a sequence identical to that set forth for Gr2940.4 in SEQ ID NO: 22,
(w) an insect receptor protein comprising consecutive amino acids having a sequence identical to that set forth for Gr2940.5 in SEQ ID NO: 23,
(x) an insect receptor protein comprising consecutive amino acids having a sequence identical to that set forth for Gr57B1 in SEQ ID NO: 46,
(y) an insect receptor protein comprising consecutive amino acids having a sequence identical to that set forth for Gr93F1 in SEQ ID NO: 48,
(z) an insect receptor protein comprising consecutive amino acids having a sequence identical to that set forth for Gr93F2 in SEQ ID NO: 49,
(aa) an insect receptor protein comprising consecutive amino acids having a sequence identical to that set forth for Gr93F3 in SEQ ID NO: 50,
(bb) an insect receptor protein comprising consecutive amino acids having a sequence identical to that set forth for Gr93F4 in SEQ ID NO: 51,
(cc) an insect receptor protein comprising consecutive amino acids having a sequence identical to that set forth for Gr94E1 in SEQ ID NO: 52,
(dd) an insect receptor protein comprising consecutive amino acids having a sequence identical to that set forth for Gr93D1 in SEQ ID NO: 53,
(ee) an insect receptor protein comprising consecutive amino acids having a sequence identical to that set forth for GrLU1=Gr36B1 in SEQ ID NO: 55,
(ff) an insect receptor protein comprising consecutive amino acids having a sequence identical to that set forth for GrLU2=Gr28A3 in SEQ ID NO: 56,
(gg) an insect receptor protein comprising consecutive amino acids having a sequence identical to that set forth for GrLU3=Gr64A1 in SEQ ID NO: 57,
(hh) an insect receptor protein comprising consecutive amino acids having a sequence identical to that set forth for GrLU7=Gr5A1 in SEQ ID NO: 59, and
(ii) an insect gustatory receptor protein which shares from 7-50% amino acid identity with any one of the proteins of (a)-(hh), and comprises seven transmembrane domains and a C-terminal domain, wherein the C-terminal domain comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

The invention provides an isolated nucleic acid molecule encoding an insect odorant receptor protein, wherein the nucleic acid molecule encodes a protein selected from the group consisting of:
(a) an insect receptor protein comprising consecutive amino acids having a sequence identical to that set forth for Gr2B1 in SEQ ID NO: 1,
(b) an insect receptor protein comprising consecutive amino acids having a sequence identical to that set forth for Gr8D1 in SEQ ID NO: 2,
(c) an insect receptor protein comprising consecutive amino acids having a sequence identical to that set forth for Gr10B1 in SEQ ID NO: 3,
(d) an insect receptor protein comprising consecutive amino acids having a sequence identical to that set forth for Gr10B2 in SEQ ID NO: 4,
(e) an insect receptor protein comprising consecutive amino acids having a sequence identical to that set forth for Gr28A2 in SEQ ID NO: 5,
(f) an insect receptor protein comprising consecutive amino acids having a sequence identical to that set forth for Gr28A4 in SEQ ID NO: 6,
(g) an insect receptor protein comprising consecutive amino acids having a sequence identical to that set forth for Gr33C1 in SEQ ID NO: 7,
(h) an insect receptor protein comprising consecutive amino acids having a sequence identical to that set forth for Gr36B2 in SEQ ID NO: 8,
(i) an insect receptor protein comprising consecutive amino acids having a sequence identical to that set forth for Gr36B3 in SEQ ID NO: 9,
(j) an insect receptor protein comprising consecutive amino acids having a sequence identical to that set forth for Gr59C1 in SEQ ID NO: 10,
(k) an insect receptor protein comprising consecutive amino acids having a sequence identical to that set forth for Gr61D1 in SEQ ID NO: 11,
(l) an insect receptor protein comprising consecutive amino acids having a sequence identical to that set forth for Gr63F1 in SEQ ID NO: 12,
(m) an insect receptor protein comprising consecutive amino acids having a sequence identical to that set forth for Gr64A2 in SEQ ID NO: 13,
(n) an insect receptor protein comprising consecutive amino acids having a sequence identical to that set forth for GR64A3 in SEQ ID NO: 14,
(o) an insect receptor protein comprising consecutive amino acids having a sequence identical to that set forth for Gr66C1 in SEQ ID NO: 15,
(p) an insect receptor protein comprising consecutive amino acids having a sequence identical to that set forth for Gr92D1 in SEQ ID NO: 16,
(q) an insect receptor protein comprising consecutive amino acids having a sequence identical to that set forth for Gr98A1 in SEQ ID NO: 17,
(r) an insect receptor protein comprising consecutive amino acids having a sequence identical to that set forth for Gr98A2 in SEQ ID NO: 18,
(s) an insect receptor protein comprising consecutive amino acids having a sequence identical to that set forth for Gr2940.1 in SEQ ID NO: 19,
(t) an insect receptor protein comprising consecutive amino acids having a sequence identical to that set forth for Gr2940.2 in SEQ ID NO: 20,
(u) an insect receptor protein comprising consecutive amino acids having a sequence identical to that set forth for Gr2940.3 in SEQ ID NO: 21,
(v) an insect receptor protein comprising consecutive amino acids having a sequence identical to that set forth for Gr2940.4 in SEQ ID NO: 22,
(w) an insect receptor protein comprising consecutive amino acids having a sequence identical to that set forth for Gr2940.5 in SEQ ID NO: 23,
(x) an insect receptor protein comprising consecutive amino acids having a sequence identical to that set forth for Gr57B1 in SEQ ID NO: 46,
(y) an insect receptor protein comprising consecutive amino acids having a sequence identical to that set forth for Gr93F1 in SEQ ID NO: 48,
(z) an insect receptor protein comprising consecutive amino acids having a sequence identical to that set forth for Gr93F2 in SEQ ID NO: 49,
(aa) an insect receptor protein comprising consecutive amino acids having a sequence identical to that set forth for Gr93F3 in SEQ ID NO: 50,
(bb) an insect receptor protein comprising consecutive amino acids having a sequence identical to that set forth for Gr93F4 in SEQ ID NO: 51,
(cc) an insect receptor protein comprising consecutive amino acids having a sequence identical to that set forth for Gr94E1 in SEQ ID NO: 52,
(dd) an insect receptor protein comprising consecutive amino acids having a sequence identical to that set forth for Gr93D1 in SEQ ID NO: 53,
(ee) an insect receptor protein comprising consecutive amino acids having a sequence identical to that set forth for GrLU1=Gr36B1 in SEQ ID NO: 55,
(ff) an insect receptor protein comprising consecutive amino acids having a sequence identical to that set forth for GrLU2=Gr28A3 in SEQ ID NO: 56,
(gg) an insect receptor protein comprising consecutive amino acids having a sequence identical to that set forth for GrLU3=Gr64A1 in SEQ ID NO: 57,
(hh) an insect receptor protein comprising consecutive amino acids having a sequence identical to that set forth for GrLU7=Gr5A1 in SEQ ID NO: 59, and
(ii) an insect odorant receptor protein which shares from 7-50% amino acid identity with any one of the proteins of (a)-(hh), and comprises seven transmembrane domains and a C-terminal domain, wherein the C-terminal domain comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

The invention provides a nucleic acid molecule comprising at least 12 nucleotides which specifically hybridizes with any of the isolated nucleic acid molecules described herein.

This invention provides a vector which comprises any of the isolated nucleic acid molecules described herein.

The invention provides a host vector system for production of a polypeptide having the biological activity of an insect gustatory or odorant receptor, which comprises any of the vectors described herein and a suitable host.

The invention provides a method of producing a polypeptide having the biological activity of an insect gustatory or odorant receptor which comprising growing any of the host vector systems described herein under conditions permitting production of the polypeptide and recovering the polypeptide so produced.

The invention provides a purified insect gustatory or odorant receptor protein encoded by any of the isolated nucleic acid molecules described herein.

The invention provides an antibody which specifically binds to an insect gustatory or odorant receptor protein encoded by any of the isolated nucleic acid molecules described herein. The invention provides an antibody which competitively inhibits the binding of any of the antibodies described herein capable of specifically binding to an insect gustatory or odorant receptor.

The invention provides a method of transforming a cell which comprises transfecting a host cell with any of the vectors described herein.

The invention provides a transformed cell produced by any of the methods described herein.

The invention provides a method of identifying a compound which specifically binds to an insect gustatory or odorant receptor which comprises contacting any of the transformed cells described herein, or a membrane fraction from said cells, with the compound under conditions permitting binding of the compound to the gustatory or odorant receptor, detecting the presence of any such compound specifically bound to the receptor, and thereby identifying the compound as a compound which specifically binds to an insect gustatory or odorant receptor.

The invention provides a method of identifying a compound which specifically binds to an insect gustatory or odorant receptor which comprises contacting any of the purified insect gustatory or odorant receptor proteins described herein with the compound under conditions permitting binding of the compound to the purified gustatory or odorant receptor protein, detecting the presence of any such compound specifically bound to the receptor, and thereby identifying the compound as a compound which specifically binds to an insect gustatory or odorant receptor.

The invention provides a method of identifying a compound which activates an insect gustatory or odorant receptor which comprises contacting any of the transformed cells described herein, or a membrane fraction from said cells, with the compound under conditions permitting activation of the gustatory or odorant receptor, detecting activation of the receptor, and thereby identifying the compound as a compound which activates an insect gustatory or odorant receptor.

The invention provides a method of identifying a compound which activates an insect gustatory or odorant receptor which comprises contacting any of the purified insect gustatory or odorant receptor proteins described herein with the compound under conditions permitting activation of the gustatory or odorant receptor, detecting activation of the receptor, and thereby identify the compound as a compound which activates an insect gustatory or odorant receptor.

The invention provides a method of identifying a compound which inhibits the activity of an insect gustatory or odorant receptor which comprises contacting any of the transformed cells described herein, or a membrane fraction from said cells, with the compound under conditions permitting inhibition of the activity of the gustatory or odorant receptor, detecting inhibition of the activity of the receptor, and thereby identifying the compound as a compound which inhibits the activity of an insect gustatory or odorant receptor.

The invention provides a method of identifying a compound which inhibits the activity of an insect gustatory or odorant receptor which comprises contacting any of the purified insect gustatory or odorant receptor proteins described herein with the compound under conditions permitting inhibition of the activity of the gustatory or odorant receptor, detecting inhibition of the activity of the receptor, and thereby identifying the compound as a compound which inhibits the activity of an insect gustatory or odorant receptor.

The invention provides a compound identified by any of the methods described herein.

The invention provides a method of combating ingestion of crops by pest insects which comprises identifying a compound by any of the methods described herein and spraying the crops with the compound.

The invention provides a method of controlling a pest population in an area which comprises identifying a compound any of the methods described herein and spraying the area with the compound.

The invention provides a composition which comprises a compound identified by any of the methods described herein and a carrier.

The invention provides a method of preparing a composition which comprises identifying a compound by any of the methods described herein, recovering the compound from the receptor protein, and admixing a carrier.

### BRIEF DESCRIPTION OF THE FIGURES

FIG. 1A-1B. The signature motif of GRs is present but diverged in members of the DOR gene family.

Sequence alignments of the complete DOR and GR gene families reveal a common amino acid motif in the putative seventh transmembrane domain of the carboxyl terminus of all GRs and 33 DORS. Alignments are shown for 23 GRs and 33 DORs (from top to bottom of FIGURE, SEQ ID NO: 61 through SEQ ID NO: 116, respectively). The average identity in the C-terminus is 29% for the GRs, 25% for the DORs, and 20% for the GRs plus DORs. Sequence relationships between the GR gene family and the DOR genes were analyzed with HMMs (Eddy, 1998), CLUSTAL alignments and neighbor joining trees (Saitou and Nei, 1987; Higgins and Sharp, 1988), and N×N BLASTP (Rubin et al., 2000) comparisons. The consensus alignment and coloring of conserved residues was assigned in ClustalX.

FIG. 2A-2B. Expression of GR genes in the proboscis and antenna

Digoxigenin-labeled antisense riboprobes derived from GR sequences hybridize to subsets of cells in adult chemosensory organs. (A) Six genes show specific hybridization to gustatory tissues. Gr47A1, Gr66C1, Gr32D1, Gr98A1, Gr28A3 and Gr33C1 are expressed in single cells within chemosensory sensilla of the proboscis labellum (data not shown for Gr28A3 and Gr33C1). (B) Three genes, Gr63F1, Gr10B1, and Gr21D1, are specifically detected in the medial aspect of the third antennal segment, the adult olfactory organ. These expression patterns were maintained in more than 50 heads for each riboprobe. Probes were annealed to sagittal sections (15 μm) of the adult fly head to assay for expression in the proboscis and to frontal sections to examine expression in the antenna.

FIG. 3. A spatial map of GR expression in the proboscis GR promoter-Gal4 transgenes drive expression in subsets of cells in the proboscis. Flies containing GR promoter-Gal4 and UAS-lacZ transgenes were examined for B-galactosidase activity staining on labial palp whole mounts. Each labial palp contains 31-36 chemosensory sensilla, arranged in approximately four rows. In the diagram of a labial palp, different rows of sensilla are depicted in different colors (adapted from Ray et al., 1993). Individual GRs show restricted expression in discrete subsets of chemosensilla. Gr47A1 is expressed in 9-11 sensilla innervating the most peripheral row of bristles, Gr32D1 is expressed in 6 sensilla innervating an intermediate row of bristles, Gr22B1 is expressed in only 3-4 sensilla innervating small bristles, and Gr66C1 and Gr28A3 are expressed in 8-10 sensilla innervating small or medium bristles. The spatial patterns for the different receptors are identical in 2-5 independent transformant lines for each promoter construct, and are also fixed among over 20 different individuals within a line.

FIG. 4A-4E. GRs are expressed in a variety of chemosensory neurons

(A, B) Expression of GFP allows visualization of dendrites and axons of neurons in the proboscis. GFP was detected in labial palp whole mounts of GR promoter-Gal4: UAS-GFP flies by direct fluorescence microscopy. Each transgene drives expression of GFP in a single bipolar neuron within a sensillum. Gr66C1 is expressed in 9 neurons (6-7 in focus) (A) and Gr22B1 is expressed in 3 neurons (B) innervating different rows of chemosensory bristles.

(C, D, E) GRs are expressed in chemosensory sensilla that reside on the internal mouthparts of the proboscis and on tarsal segments of legs. In addition to expression in the proboscis labellum, Gr32D1, Gr66C1 and Gr28A3 are also detected in the cibarial organs of the mouth. (C) LacZ expression in a whole mount proboscis is illustrated for the Gr66C1-Gal4: UAS-lacZ line. The arrow denotes the cibarial organ. (D) One transgenic line, Gr2B1-Gal4, drives expression exclusively in the labral sense organ of the mouth, and not in the cibarial organs or in the labellum of the proboscis. The arrow denotes the labral sense organ. (E) Gr32D1 is expressed in the proboscis labellum and in the cibarial organs. In addition, Gr32D1-Gal4 drives expression of GFP in 2-3 neurons in the fourth and fifth tarsal segments of all legs. Receptor expression was examined by B-galactosidase activity staining of GR promoter-Gal4: UAS-lacZ flies (C, D) or by fluorescent visualization of GR promoter-Gal4: UAS-GFP flies (E).

FIG. 5A-5G. GRs are expressed in larval chemosensory neurons

(A) The antenno-maxillary complex of larvae is a bilaterally symmetric structure containing the dorsal organ mediating smell and the terminal organ involved in both taste and smell. Shown is the anterior ventral region of a larva viewed by differential interference contrast. On one half of the larval head, the sensilla of the terminal organ is outlined with black dotted lines and the pore of the terminal organ is denoted by an outlined arrow. The dome of the dorsal organ is denoted by a filled arrowhead.

(B-E) Gr32D1, Gr66C1, and Gr28A3 are expressed in the proboscis labellum in the adult (FIG. 3), and are expressed in a single bilaterally symmetric neuron in the terminal organ of larvae (B, E, data not shown). Gr2B1 is expressed in the labral sense organ of the adult proboscis, and is expressed in two neurons innervating the dorsal organ (filled arrow), one neuron innervating the terminal organ (outlined arrow), and one neuron innervating the ventral pits in each of the thoracic segments in larvae (C). Gr21D1 is expressed in the adult antenna and in a single larval neuron innervating the terminal organ (D). The dome of the dorsal organ is autoflourescent.

(F,G) Different GRs are expressed in distinct chemosensory neurons. In larvae bearing two GR promoter-Gal4 fusions and UAS-GFP, two GFP positive cells per terminal organ are observed. The different promoter combinations illustrated are Gr21D1-Gal4 plus Gr66C1-Gal4 (F) and Gr32D1-Gal4 plus Gr66C1-Gal4 (G). The pseudotracheae of the larval mouth shows autoflourescence.

FIG. 6A-6H. Axonal Projections of Larval Chemosensory Neurons

Projections of neurons bearing different GRs are spatially segregated in the larval brain. In all panels, whole mount larval brains from GR promoter-Gal4: UAS-nSyb-GFP flies were stained with anti-GFP to label axonal termini (green), mAb nc82 to label neuropil (red), and TOTO-3 to counterstain nuclei (blue). Each image represents a composite of 1 um optical sections through the larval brain, encompassing the terminal projections. Projections extend 5-10 um in depth for B, C, D, G and 10-20 um in depth for E, F, G.

(A) The larval brain is composed of the two dorsal brain hemispheres (BH) and the ventral hindbrain (HB). The subesophageal ganglion (SOG) resides in the hindbrain, at the juncture of the hindbrain with the brain hemispheres. The antennal lobe (AL) is a small neuropil on the anterior edge of the brain hemisphere (denoted with an arrow in panel E, G).

(B-D) GR-bearing neurons project to discrete locations in the larval brain. Gr32D1 is expressed in the proboscis in the adult and in one neuron in the terminal organ in larvae. In Gr32D1-Gal4:UAS-nSyb-GFP larval brains, a single terminal arborization is observed in the SOG (C). A similar pattern is observed for neurons expressing Gr66C1, a gene expressed in the adult proboscis and in a single neuron in the terminal organ and two in the mouth of larvae (B, D). Panels D is a higher magnification (3×) of Panel B.

(E) Projections of gustatory neurons from different body regions are spatially segregated in the fly brain. Gr2B1 is expressed in two neurons innervating the dorsal organ, one neuron innervating the terminal organ, and one neuron innervating the ventral pits. Axons from ventral pit neurons enter the hindbrain via thoracic nerves and terminate in the antennal lobe (arrows), in a location that is distinct from the termini of other Gr2B1-bearing neurons.

(F) Segregation is less apparent in the terminal projections of two different taste receptors. Larvae that contain Gr66C1-Gal4 and Gr32D1-Gal4 along with UAS-nSyb-GFP reveal two partially overlapping projection patterns.

(G,H) Distinct projection patterns are observed for the two different chemosensory modalities, taste and smell. Gr21D1 is expressed in the adult antenna and in a single neuron in the terminal organ of larvae. Gr21D1 axons enter the antennal lobe (arrows) (G). In larvae that contain Gr21D1-Gal4 and Gr66C1-Gal4 along with UAS-nSyb-GFP, two discrete termini are apparent, one entering the SOG, and a second entering the antennal lobe (H).

FIG. 7A-7C. A subset of GRs encode olfactory receptors

GR-bearing neurons in the antenna project to discrete glomeruli in the antennal lobe. Adult transgenic flies in which Gr21D1 promoter-Gal4 drives expression of UAS-lacZ (A) or UAS-GFP (B) show specific labelling in subsets of cells in the medial aspect of the antenna. This expression pattern resembles that determined for the endogenous gene. LacZ expression was detected in 15 um frontal sections of the antenna (A); GFP expression was examined in whole antennae (B).

(C) Gr21D1-bearing neurons project to a single bilaterally symmetric glomerulus on the ventral-most region of the antennal lobe. Whole mount brains of Gr21D1-Gal4: UAS-nSyb-GFP flies were examined by fluorescent immunohistochemistry, with anti-GFP to visualize axonal termini of Gr21D1-bearing neurons (green), mAb nc82 to label brain neuropil (red), and TOTO-3 to counterstain nuclei (blue). Gr21D1-bearing neurons send projections to the V glomerus in the antennal lobe (Stocker et al., 1990; Laissue et al., 1999) and do not project to the subesophageal ganglion (located in the bottom part of C).

### DETAILED DESCRIPTION OF THE INVENTION

Throughout this application, the following standard abbreviations are used to indicate specific amino acids:

| 3-character | | 1-character |
|---|---|---|
| abbreviation | Amino Acid | abbreviation |
| Ala | Alanine | A |
| Arg | Arginine | R |
| Asn | Asparagine | N |
| Asp | Aspartic Acid | D |
| Cys | Cysteine | C |
| Gln | Glutamine | Q |
| Glu | Glutamic Acid | E |
| Gly | Glycine | G |
| His | Histidine | H |
| Ile | Isoleucine | I |
| Leu | Leucine | L |
| Lys | Lysine | K |
| Met | Methionine | M |
| Phe | Phenylalanine | F |
| Pro | Proline | P |
| Ser | Serine | S |
| Thr | Threonine | T |
| Trp | Tryptophane | W |
| Tyr | Tyrosine | Y |
| Val | Valine | V |
| Asx | Asparagine/ | B |
| | Aspartic Acid | |
| Glx | Glutamine/ | Z |
| | Glutamic Acid | |
| *** | (End) | * |
| Xxx | Unidentified, any, | X |
| | or as specified. | |

Throughout this application, the following standard abbreviations are used to indicate specific nucleotides:

| | |
|---|---|
| C = cytosine | A = adenosine |
| T = thymidine | G = guanosine. |

This invention provides a family of isolated nucleic acid molecules encoding insect gustatory and odorant receptors. In one embodiment, the receptor is a gustatory receptor. In one embodiment, the receptor is an odorant receptor.

The family of receptors comprises:

Newly identified receptors disclosed herein comprise:

Gr2B1 (SEQ ID NO: 1)
MDTLRALEPLHPACQVCNLWPWRLAPPPDSEGILLRRSRWLELYGWTVLI
AATSFTVYGLFQESSVEEKQDSESTISSIGHTVDFIQLVGMRVAHLAALL
EALWQRQAQRGFFAELGEIDRLLSKALRVDVEAMRINMRRQTSRRAVWIL
WGYAVSQLLILGAKLLSRGDRFPIYWISYLLPLLVCGLRYFQIFNATQLV
RQRLDVLLVALQQLQLHQKGPAVDTVLEEQEDLEEAAMDRLIAVRLVYQR
VWALVALLNRCYGLSMLMQVGNDFLAITSNCYWMFLNFRQSAASPFDILQ
IVASGVWSAPHLGNVLVLSLLCDRTAQCASRLALCLHQVSVDLRNESHNA
LITQFSLQLLHQRLHFSAAGFFNVDCTLLYTIVGATTTYLIILIQFHMSE
STIGSDSNGQ
Gr8D1 (SEQ ID NO: 2)
MSGHLGRVLQFHLRLYQVLGFHGLPLPGDGNPARTRRRLMAWSLFLLISL
SALVLACLFSGEEFLYRGDMFGCANDALKYVFAELGVLAIYLETLSSQRH
LANFWWLHFKLGGQKTGLVSLRSEFQQFCRYLIFLYAMMAAEVAIHLGLW
QFQALTQHMLLFWSTYEPLVWLTYLRNLQFVLHLELLREQLTGLEREMGL
LAEYSRFASETGRSFPGFESFLRRRLVQKQRIYSHVYDMLKCFQGAFNFS
ILAVLLTINIRIAVDCYFMYYSIYNNVINNDYYLIVPALLEIPAFIYASQ
SCMVVVPRIAHQLHNIVTDSGCCSCPDLSLQIQNFSLQLLHQPIRIDCLG
LTILDCSLLTRMACSVGTYMIYSIQFIPKFSNTYM
Gr10B1 (SEQ ID NO: 3)
MQRTHLEFEFKNAPQEPKRPFEFFMYFKFCLINLMMMIQVCGIFAQYGEV
GKGSVSQVRVHFAIYAFVLWNYTENMADYCYFINGSVLKYYRQFNLQLGS
LRDEMDGLRPGGMLLHHCCELSDRLEELRRRCREIHDLQRESFRMHQFQL
IGLMLSTLINNLTNFYTLFHMLAKQSLEEVSYPVVVGSVYATGFYIDTYI
VALINEHIKLELEAVALTMRRFAEPREMDERLTREVRNKIFSFLATTLEI
MIQIWLSFFANFDDVTPYRKCENRPKNLFFKIRQKVIGIVSSGKLKLLVS
LRFFIIDNRLILNLHKYLAIKLNFLNLIQIEHLSLELLNYQPPMLCGLLH
LDRRLVYLIAVTAFSYFITLVQFDLYLRKKS
Gr10B2 (SEQ ID NO: 4)
MRVGKLCRLALRFWMGLILVLGFSSHYYNPTRRRLVYSRILQTYDWLLMV
INLGAFYLYYRYAMTYFLEGMFRRQGFVNQVSTCNVFQQLLMAVTGTWLH
FLFERHVCQTYNELSRILKHDLKLKEHSRFYCLAFLAKVYNFFHNFNFAL
SAIMHWGLRPFNVWDLLANLYFVYNSLARDAILVAYVLLLLNLSEALRLN
GQQEHDTYSDLMKQLRRRERLLRIGRRVHRMFAWLVAIALIYLVFFNTAT
IYLGYTMFIQKHDALGLRGRGLKMLLTVVSFLVILWDVVLLQVICEKLLA
EENKICDCPEDVASSRTTYRQWEMSALRRAITRSSPENNVLGMFRMDMRC
AFALISCSLSYGIIIIQIGYIPG
Gr28A2 (SEQ ID NO: 5)
MAFKLWERFSQADNVFQALRPLTFISLLGLAPFRLNLNPRKEVQTSKFSF
FAGIVHFLFFVLCFGISVKEGDSIIGYFFQTNITRFSDGTLRLTGILAMS
TIFGFANFKRQRLVSIIQNNIVVDEIFVRLGMKLDYRRILLSSFLISLGM
LLFNVIYLCVSYSLLVSATISPSFVTFTTFALPHINISLMVFKFLCTTDL
ARSRFSMLNEILQDILDAHIEQLSALELSPMHSVVNHRRYSHRLRNLIST
PMKRYSVTSVIRLNPEYAIKQVSNIHNLLCDICQTIEEYFTYPLLGIIAI
SFLFILFDDFYILEAILNPKRLDVFEADEFFAFFLMQLIWYIVIIVLIVE
GSSRTILHSSYTAAIVHKILNITDDPELRDRLFRLSLQLSHRKVLFTAAG
LFRLDRTLIFTVN FLQITGAATCYLIILIQF
Gr28A4 (SEQ ID NO: 6)
MIRCGLDIFRGCRGRFRYWLSARDCYDSISLMVAIAFALGITPFLVRRNA
LGENSLEQSWYGFLNAIFRWLLLAYCYSYINLRNESLIGYFMRNHVSQIS
TRVHDVGGIIAAVFTFILPLLLRKYFLKSVKNMVQVDTQLERLRSPVNFN
TVVGQVVLVILAVVLLDTVLLTTGLVCLAKMEVYASWQLTFIFVYELLAI
SITICMFCLMTRTVQRRITCLHKFDFATMSALRRVRKYFISSQVYEALRP
LFFLTFLYGLTPFHVVRRKMGESYLKMSCFGVFNIFIYICLCGFCYISSL
RQGESIVGYFFRTEISTIGDRLQIFNGLIAGAVIYTSAILKRCKLLGTLT
ILHSLDTNFSNIGVRVKYSRIFRYSLLVLIFKLLILGVYFVGVFRLLVSL
DVTPSFCVCMTFFLQ
Gr33C1 (SEQ ID NO: 7)
MKRKAVEVIGLIPLNRQQSETNFILDYAMMCIVPIFYVACYLLINLSHII
GLCLLDSCNSVCKLSSHLFMHLGAFLYLTITLLSLYRRKEFFQQFDARLN
DIDAVIQKCQRVAEMDKVKVTAVKHSVAYHFTWLFLFCVFTFALYYDVRS
LYLTFGNLAFIPFMVSSFPYLAGSIIQGEFIYHVSVISQRFEQINMLLEK
INQEARHRHAPLTVFDIESEGKKERKTVTPITVMDGRTTTGFGNENKFAG
EMKRQEGQQKNDDDDLDTSNDEDEDDFDYDNATIAENTGNTSEANLPDLF
KLHDKILALSVITNGEFGPQCVPYMAACFVVSIFGIFLETKVNFIVGGKS
RLLDYMTYLYVIWSFTTMMVAYIVLRLCCNANNHSKQSAMIVHEIMQKKP
AFMLSNDLFYNKMKSFTLQFLHWEGFFQFNGVGLFALDYTFIFSTVSAAT
SYLIVLLQFDMTAILRNEGLMS
Gr36B2 (SEQ ID NO: 8)
MVDWVVLLLKAVHIYCYLIGLSNFEFDCRTGRVFKSRRCTIYAFMANIFI
LITIIYNFTAHGDTNLLFQSANKLHEYVIIIMSGLKIVALITVLNRWLQR
GQMMQLVKDVIRLYMINPQLKSMIRWGILLKAFISFAIELLQVTLSVDAL
DRQGTAEMMGLLVKLCVSFIMNLAISQHFLVILLIRAQYRIMNAKLRMVI
EESRPLSFLQLRNGAFMTRCCYLSDQLEDIGEVQSQLQSMVGQLDEVFGM
QGLMAYSEYYLSIVGTSYMSYSIYKYGPHNLKLSAKTSIIVCILITLFYL
DALVNCNNMLRVLDHHKDFLGLLEERTVFASSLDIRLEESVSFESLQLQL
ARNPLKINVMGMFPITRGSTAAMCASVIVNSIFLIQFDME
Gr36B3 (SEQ ID NO: 9)
MDLESFLLGAVYYYGLFIGLSNFEFDWNTGRVFTKKWSTLYAIALDSCIF
ALYIYHWTGNTNIVNAIFGRANMLHEYVVAILTGLRIVTGLFTLILRWYQ
RCKMMDLASKVVRMYVARPQVRRMSRWGILTKFIFGSITDGLQMAMVLSA
MGSRVDSQFYLGLGLQYWMFVILNMAMMQQHMIMLFVRTQFQLINTELRQ
VIDEAKDLLLSPRHQGVFMTKCCSLADQIENIARIQSQLQTIMNQMEEVF
GIQGAMTYGGYYLSSVGTCYLASILKHGYENLSMTLSTVILAYSWCFFYY
LDGMLNLSVMLHVQDDYWEMLQILGKRTIFVGLDVRLEEAVST
Gr59C1 (SEQ ID NO: 10)
MIKLYFRYSLAIGITSQQFSNRKFFSTLFSRTYALIANIVTLIMLPIVMW
QVQLVFQQKKTFPKLILITNNVREAVSFLVILYTVLSRGFRDTAFKEMQP
LLLTLFREEKRCGFKGIGGVRRSLRILLFVKFFTLSWLCVTDVLFLLYST
DALIWVNVLRFFFKCNTNNILEMVPMGYFLALWHIARGFDCVNRRLDQIV
KSKSTRKHRELQHLWLLHACLTKTALNINKIYAPQMLASRFDNFVNGVIQ
AYWGAVFTFDLSTPFFWVVYGSVQYHVRCLDYYLIDNMCDVAVEYHDSAK
HSWSEVRWTKEVSAFGSILLYICMLMQLLSFQISSYVIYANSTKLQLWSC
GLFQANRSMWFAMISSVLYYILVLLQFHLVMRK*
Gr61D1 (SEQ ID NO: 11)
MSRTSDDIRKHLKVRRQKQRAILAMRWRCAQGGLEFEQLDTFYGAIRPYL
CVAQFFGIMPLSNIRSRDPQDVKFKVRSIGLAVTGLFLLLGGMKTLVGAN
ILFTEGLNAKNIVGLVFLIVGMVNWLNFVGFARSWSHIMLPWSSVDILML
FPPYKRGKRSLRSKVNVLALSVVVLAVGDHMLYYASGYCSYSMHILQCHT
NHSRITFGLYLEKEFSDIMFIMPFNIFSMCYGFWLNGAFTFLWNFMDIFI
VMTSIGLAQRFQQFAARVGALEGRHVPEALWYDIRRDHIRLCELASLVEA
SMSNIVFVSCANNVYVICNQALAIFTKLRHPINYVYFWYSLIFLLARTSL
VFMTASKIHDASLLPLRSLYLVPSDGWTQEVQRFADQLTSEFVGLSGYRL
FCLTRKSLFGMLATLVTYELMLLQIDAKSHKGLRCA
Gr63F1 (SEQ ID NO: 12)
MRPSGEKVVKGHGQGNSGHSLSGMANYYRRKKGDAVFLNAKPLNSANAQA
YLYGVRKYSIGLAERLDADYEAPPLDRKKSSDSTASNNPEFKPSVFYRNI
DPINWFLRIIGVLPIVRHGPARAKFEMNSASFIYSVVFFVLLACYVGYVA
NNRIHIVRSLSGPFEEAVIAYLFLVNILPIMIIPILWYEARKIAKLFNDW
DDFEVLYYQISGHSLPLKLRQKAVYIAIVLPILSVLSVVITHVTMSDLNI
NQVVPYCILDNLTAMLGAWWFLICEAMSITAHLLAERFQKALKHIGPAAM
VADYRVLWLRLSKLTRDTGNALCYTFVFMSLYLFFIITLSIYGLMSQLSE
GFGIKDIGLTITALWNIGLLFYICDEAHYASVNVRTNFQKKLLMVELNWM
NSDAQTEINMFLRATEMNPSTINCGGFFDVNRTLFKGLLTTMVTYLVVLL
QFQISIPTDKGDSEGANNITVVDFVMDSLDNDMSLMGASTLSTTTVGTTL
PPPIMKLKGRKG
Gr64A2 (SEQ ID NO: 13)
MPVRKVSSKFAEDLTFTWFSVRSYYALVTILFFGVSSGYMVAFVTSVSFN
FDSVETLVFYLSIFLISLSFFQLARKWPEIAQSWQLVEAKLPPLKLPKER
RSLAQHINMITTVATTCSLVEHIMSMLSMGYYVNSCPRWPDRPIDSFLYL
SFSSVFYFVDYTRFLGIVGKVVNVLSTFAWNFNDIFVMAVSVALAARFRQ
LNDYMMREARLPTTVDYWMQCRINFRNLCKLCEEVDDAISTITLLCFSNN
LYFICGKILKSMQAKPSIWHALYFWFSLVYLLGRTLILSLYSSSINDESK
RPLVIFRLVPREYWCDELKRFSEEVQMDNVALTGMKFFRLTRGVVISVAG
TIVTYELILLQFNGEEK
Gr64A3 (SEQ ID NO: 14)
MELSRSDKEAFLSDGSFHQAVGRVLLVAEFFAMMPVKGVTGKHPSDLSFS
WRNIRTCFSLLFIASSLANFGLSLFKVLNNPISFNSIKPIIFRGSVLLVL
IVALNLARQWPQLMMYWHTVEKDLPQYKTQLTKWKMGHTISMVMLLGMML
SFAEHILSMVSAINYASFCNRTADPIQNYFLRTNDEIFFVTSYSTTLALW
GKFQNVFSTFIWNYMDLFVMIVSIGLASKFRQLNDDLRNFKGMNMAPSYW
SERRIQYRNICILCDKMDDAISLITMVSFSNNLYFICVQLLRSLNTMPSV
AHAVYFYFSLIFLIGRTLAVSLYSSSVHDESRLTLRYLRCVPKESWCPEV
KRFTEEVISDEVALTGMKFFHLTRKLVLSVAGTIVTYELVTLIQFHEDND
LWDCDQSYYS
Gr66C1 (SEQ ID NO: 15)
MDNMAQAEDAVQPLLQQFQQLFFISKIAGILPQDLEKFRSRNLLEKSRNC
MIYMLSTLILYVVLYNILIYSFGEEDRSLKASQSTLTFVIGLFLTYIGLI
MMVSDQLTALRNQGRIGELYERIRLVDERLYKEGCVMDNSTIGRRIRIML
IMTVIFELSILVSTYVKLVDYSQWMSLLWIVSAIPTFINTLDKIWFAVSL
YALKERFEAINATLEELVDTHEKHKLWLRGNQEVPPPLDSSQPPQYDSNL
EYLYKELGAIDAASRKPPPPPATNMVHESELGNAAKVEEKLNNLCQVHDE
ICEIGKALNELWSYPILSLMAYGFLIFTAQLYFLYCATQYQSIPSLFRSA
KNPFITVIVLSYTSGKCVYLIYLSWKTSQASKRTGISLHKCGVVADDNLL
YEIVNHLSLKLLNHSVDFSACGFFTLDMETLYGVSGGITSYLIILIQFNL
AAQQAKEAIQTFNSLNDTAGLVGAATDMDNISSTLRDFVTTTMTPAV
Gr92D1 (SEQ ID NO: 16)
MFEFLHQMSAPKLSTSILRYIFRYAQFIGVIFFCLHTRKDDKTVFIRNWL
KWLNVTHRIITFTRFFWVYIASISIKTNRVLQVLHGMRLVLSIPNVAVIL
CYHIFRGPEIIDLINQFLRLFRQVSDLFKTKTPGFGGRRELILILLNLIS
FAHEQTYLWFTIRKGFSWRFLIDWWCDFYLVSATNIFIHINSIGYLSLGV
LYSELNKYVYTNLRIQLQKLNTSGSKQKIRRVQNRLEKCISLYREIYHTS
IMFHKFVPLLFLALIYKVLLIALIGFNVAVEFYLNSFIFWILLGKHVLDL
FLVTVSVEGAVNQFLNIGMQFGNVGDLSKFQTTVSQFIFIDFIPI
Gr98A1 (SEQ ID NO: 17)
MVAQKSRLLARAFPYLDIFSVFALTPPPQSFGHTPHRRLRWYLMTGYVFY
ATAILATVFIVSYFNIIAIDEEVLEYNVSDFTRVMGNIQKSLYSIMAIAN
HLNMLINYRRLGGIYKDIADLEMDMDEASQCFGGQRQRFSFRFRMALCVG
VWMILMVGSMPRLTMTAMGPFVSTLLKILTEFVMIMQQLKSLEYCVFVLI
IYELVLRLRRTLSQLQEEFQDCEQQDMLQALCVALKRNQLLLGRIWRLEG
DVGSYFTPTMLLLFLYNGLTILHMVNWAYINKFLYDSCCQYGPEYCLFVL
LVYELILRTRHVLEQLKDDLEDFDCGARIQELCVTLKQNQLLIGRIWRLV
DEIGAYFRWSMTLLFLYNGLTILHVVNWAIIRSIDPNDCCQLMSFHFSLN
MEANRSRLLAAARPYIQIYSIFGLTPPIQFFTRTLHKRRGIVILGYACYL
ISISLMVIYECYANIVALQKDIHKFHAEDSSKVMGNTQKVLVVAMFVWNQ
LNILLNFRRLARIYDDIADLEIDLNNASSGFVGQRHWWRFRFRLALSVGL
WIVLLVGLTPRFTLVALGPYLHWTNKVLTEIILIMLQLKCTEYCVFVLLI
YELILRGRHILQQISVELEGNQSRDSVQELCVALKRNQLLAGRIWGLVNE
VSLYFTLSLTLLFLYNELTILQIVNWALIKSVNPNECCQYTEDYLILKMG
LREYSLQMEHLKLIFTCGGLFDINLKFFGGVKLKL
Gr98A2 (SEQ ID NO: 18)
MEAKRSRLLTTARPYLQVLSLFGLTPPAEFFTRTLRKRRRFCWMAGYSLY
LIAILLMVFYEFHANIVSLHLEIYKFHVEDFSKVMGRTQKFLIVAIATCN
QLNILLNYGRLGLIYDEIANLDLGIDKSSKNFCGKSHWWSFRLRLTLSIG
LMWVIIIGVIPRLTLGRAGPFFHWVNQVLTQIILIMLQLKGPEYCLFVLL
VYELILRTRHVLEQLKDDLEDFDCGARIQELCVTLKQNQLLIGRIWRLVD
EIGAYFRWSMTLLFLYNGLTILHVVAIIRSIDPNDCCQLSEE
Gr2940.1 (SEQ ID NO: 19)
MFRPSGSGYRQKWTGLTLKGALYGSWILGVFPFAYDSWTRTLRRSKWLIA
YGFVLNAAFILLVVTNDTESETPLRMEVFHRNALEQINGIHDIQSLSMVS
IMLLRSFWKSGDIERTLNELEDLQHRYFRNYSLEECISFDRFVLYKGFSV
VLELVSMLVLELGMSPNYSKQFFIGLGSLCLMLLAVLLGASHFHLAVVFV
YRYVWIVNRELLKLVNKMAIGETVESERMDLLLYLYHRLLDLGQRLASIY
DYQMVMVMVSFLIANVLGIYFFIIYSISLNKSLDFKILVFVQALVINMLD
FWLNVEICELAERTGRQTSTILKLFNDIENIDEKLERSVSFTSQHYCETD
FALFCSHRRLRFHHCGLFYVNYEMGFRMAITSFLYLLFLIQFDYWNL
Gr2940.2 (SEQ ID NO: 20)
MVKQAEDREHGIMLDVFQRNALLYQISSLMGVVGVVSICTVHLRTLWRSK
HLEEIYNGLMLLEAKYFCSNAVECPAFDGYVIQKGVVIVVGLLAPWMVHF
GMPDSKLPVLNVLVVSMVKLGTLLLALHYHLGVVIIYRFVWLINRELLSL
VCSLRGNHKGSSSRVRFLLKLYNKLVNLYSKLADCYDCQTVLMMAIFLAA
NIIVCFYMIVYRISLSKMSFFVMLIMFPLAIANNFMDFWLSMKVCDLLQK
TGRQTSMILKLFNDIENMDKDLEISISDFALYCSHRRFKFLHCGLFHVNR
EMGFKMFVASVLYLLYLVQF
Gr2940.3 (SEQ ID NO: 21)
MFASRSDLQSRLCWIILKATLYSSWFLGVFPYRFDSRNGQLKRSRFLLFY
GLILNFFLLLKMVCSGGQKLGIPEAFARNSVLENTHYTTGMLAVFSCVVI
HFLNFWGSTRVQDLANELLVLEYQQFASLNETKCPKFNSFVIQKWLSVIG
LLLSYLSIAYGLPGNNFSVEMVLINSLVQFSFNCNIMYYYIGVLLIYRYL
WLINGQLLEMVTNLKLDCSVDSSRIRKYLSLYRRLLELKGYMVATYEYHM
TLVLTTGLASNFLAIYSWIVLDISMNINFIYLLIFPLFLLVNVWNLWLSI
AASDLAENAGKSTQTVLKLFADLEVKDIELERSVSVNSNRYKQVNEFALL
CGHCQFNFHVCGLFTINYKNGFQMIITSFLYLIYMIQFD
Gr2940.4 (SEQ ID NO: 22)
MINVVIGIINVLSALIVHFMNFWGSRKVGEICNELLILEYQDFEGLNGRN
CPNFNCFVIQKCLTILGQLLSFFTLNFALPGLEFHICLVLLSCLMEFSLN
LNIMHYHVGVLLIYRYVWLINEQLKDLVSQLKLNPETDFSRIHQFLSLYK
RLLELNRKLVIAYEYQMTLFIIAQLSGNIVVIYFLIVYGLSMRTYSIFLV
AFPNSLLINIWDFWLCIAACDLTEKAGDETAIILKIFSDLEHRDDKLEKF
RFQLCGLFSMNCRMGFKMITTFLYLVYLVQFDYMNL*
Gr2940.5 (SEQ ID NO: 23)
MSQPKRIHRICKGLARFTIPATLYGSWVLGLFPFTFDSRKRRLNRSKWLL
AYGLVLNLTLLVLSMLPSTDDHNSVKVEVFQRNPLVKQVEELVEVISLIT
TLVTHLRTFSRSSELVEILNELLVLDKNHFSKLMLSECHTFNRYVIEKGL
VIILEIGSSLVLYFGIPNSKIVVYEAVCIYIVQLEVLMVVMHFHLAVIYI
YRYLWIINGQLLDMASRLRRGDSVDPDRIQLLLWLYSRLLDLNHRLTAIY
DIQVTLFMATLFSVNIIVGHVLVICWINITRFSLLVIFLLFPQALIINFW
DLWQGIAFCDLAESTGKKTSMILKLFNDMENMDQETERRVSEYMFQNLMY
FKYFKHPLIFVAEFTLFCSHRRLKVCHLGLLDINYEMGFRMIITNILYVV
FLVQFDYMNL

Previously reported Gustatory Receptors which are family members:

a) Full-length clones

Gr21D1 (SEQ ID NO: 24)
MGVMPIHRNPPEKNLPRTGYSWGSKQVMWAIFIYSCQTTIVVLVLRERVK
KFVTSPDKRFDEAIYNVIFISLLFTNFLLPVASWRHGPQVAIFKNMWTNY
QYKFFKTTGSPIVFPNLYPLTWSLCVFSWLLSIAINLSQYFLQPDFRLWY
TFAYYPIIAMLNCFCSLWYINCNAFGTASRALSDALQTTIRGEKPAQKLT
EYRHLWVDLSHMMQQLGRAYSNMYGMYCLVIFFTTIIATYGSISEIIDHG
ATYKEVGLFVIVFYCMGLLYIICNEAHYASRKVGLDFQTKLLNINLTAVD
AATQKEVEMLLVAINKNPPIMNLDGYANINRELITTNISFMATYLVVLLQ
FKITEQRRIGQQQA
Gr22B1 (SEQ ID NO: 25)
MFQPRRGFSCHLAWFMLQTTLYASWLLGLFPFTFDSRRKQLKRSRWLLLY
GFVLHSLAMCLAMSSHLASKQRRKYNAFERNPLLEKIYMQFQVTTFFTIS
VLLLMNVWKSNTVRKIANELLTLEGQVKDLLTLKNCPNFNCFVIKKHVAA
IGQFVISIYFCLCQENSYPKILKILCCLPSVGLQLIIMHFHTEIILVYRY
VWLVNETLEDSHHLSSSRIHALASLYDRLLKLSELVVACNDLQLILMLII
YLIGNTVQIFFLIVLGVSMNKRYIYLVASPQLIINFWDFWLNIVVCDLAG
KCGDQTSKVLKLFTDLEHDDEELERSLNEFAWLCTHRKFRFQLCGLFSIN
HNMGFQMIITSFLYLVYLLQFDFMNLC
Gr23A1a (SEQ ID NO: 26)
MKTLECLTRRFLEVIFSVLALVPLPPISQLGWLFLSLAIRCCWIVYFIYL
LDVAISFSWVAIENVGNAVGTMLFVGNSVLGPALLLESVLKQKTHSQLED
LRVQTELQLQRLGMFGRSRHAAYLLPLIGVQFTCDLVRLATNFGETVSPV
FCISLPLMWLLRYRYVQLVQHVMDLNQRSIHLRRSLLSMASGNDLWQPYG
VQECLQLQTLRTTYERIFECYETFSDCYGWGMLGLRLLTSFQFVTNAYWM
IMGIYDGGNVRSLIFNGATGIDFGTPIATLFWHGDSGAENGRQIGCLISK
LVKPQCSKLYNDLVSEFSLQTLHQRFVVTAKDFFSLNLHLLSSMFAAVVT
YLVILIQFMFAERSSTRGSG
Gr23A1b (SEQ ID NO: 27)
MFPPTRVQASSRVVLKIFHFILVAFSLRSRRLSRLVLWLQFLGWLTWFIS
MWTQSVIYAQTIDCTLDCSLRHILTFFQTVSHAFIVVTSFLDGFRIKQDQ
LDEPIAFEDSDPWLAFTVLAMLVPTLGVEYLVCSNAPEYAFRIRIYHLKT
LPSFLALQVIISFILEVMKVNIRVRQTKLQLLILARELSCRWPQRKQKPQ
FSDQQAHRVKDLKRRYNDLHYLFVRINGYFGGSLLTIIIVHFAIFVSNSY
WLFVDIRTRPWRIYAILLNLGFIFNVALQMAAACWHCQQSYNLGRQIGCL
ISKLVKPQGSKLYNDLVSEFSLQTLHQRFVVTAKDFFSLNLHLLSSMFAA
VVTYLVILIQFMFAERSSTRGSG
Gr32D1 (SEQ ID NO: 28)
MPIYEQVSDYEVGPPTKTNEFYSFFVRGVVHALTIFNVYSLFTPISAQLF
FSYRETDNVNQWIELLLCILTYTLTVFVCAHNTTSMLRIMNEILQLDEEV
RRQFGANLSQNFGFLVKFLVGITACQAYIIVLKIYAVQGEITPTSYILLA
FYGIQNGLTATYIVFASALLRIVYIRFHFINQLLNGYTYGQQHRRKEGGA
RARRQRGDVNPNVNPALMEHFPEDSLFIYRMHNKLLRIYKGINDCCNLIL
VSFLGYSFYTVTTNCYNLFVQITGKGMVSPNILQWCFAWLCLHVSLLALL
SRSCGLTTTEVSNYIGDKISIFMSVFISRPMPHPKFLQGCMPSRRSIRIS
GFHYQTDKFLTKSIKQEVQFTAYGFFAIDNSTLFKIFSAVTTYLVTLIQF
KQLEDSKVEDPVPEQT
Gr39D1 (SEQ ID NO: 29)
MLYSFHPYLKYFALLGLVPWSESCAQSKFVQKVYSAILIILNAVHFGISI
YFPQSAELFLSLMVNVIVPVARIVCVTVIILQVMVHYDDYFRFCREMKYL
GLRLQCELKIHVGRLKWQSYAKILALGIGFLVTVLPSIYVALSGSLLYFW
SSLLSILIIRMQFVLVLLNVELLGHHVSLLGIRLQNVLECHLMGANCTLD
GNANRLCSLEFLLALKQSHMQLHYLFTHFNDLFGWSILGTYVVLFSDSTV
NIYWTQQVLVEVYEYKYLYATFSVFVPSFFNILVFCRCGEFCQRQSVLIG
SYLRNLSCHPSIGRETSYKDLLMEFILQVEQNVLAINAEGFMSTDNSLLM
SILAAKVTYLIVLMQFSSV
Gr39D2a (SEQ ID NO: 30)
MGTRNRKLLFFLHYQRYLGLTNLDFSKSLHIYWLHGTWSSTAIQIVVVGV
FMAALLGALAESLYYMETKSQTGNTFDNAVILTTSVTQLLANLWLRSQQK
SQVNLLQRLSQVVELLQFEPYAVPQFRWLYRIWLLVCLIYGAMVTHFGIN
WLTTMQISRVLTLIGFVYRCVLANFQPTCYTGMVVILKKLLQVQVKQLEH
LVSTTTISMAGVAGCLRTHDEILLLGQRELIAVYGGVILFLFIYQVMQCI
LIFYISNLEGFHSSNDLVLIFCWLAPMLPYLILPLVVNDIHNQANKTAKM
LTKVPRTGTGLDRMIEKFLLKNLRQKPILTAYGFFALDKSTLFKLFTAIF
TYMVILVQFKEMENSTKSINKF
Gr39D2b (SEQ ID NO: 31)
MDFQPGELCAYYRLCRYLGIFCIDYNPTKKKFRLRRSVLCYIVHFALQAY
LVGCISVMVTYWRRCFKSELTTTGNHFDRLVMVIALGILVVQNAWLIWLQ
APHLRIVRQIEFYRRNHLANVRLLLPKRLLWLIIATNVVYMANFIKTCIF
EWLTDASRLFVITSLGFPLRYLVTSFTMGTYFCMVHIVRLVLDWNQSQIN
AIIDESADLKMTSPNRLRLRVCLEMHDRLMLLCNDEISLVYGFIAWLSWM
FASLDVTGVIYLTMVIQTKKSIVLKLITNVVWLSPTFMTCAASFMSNRVT
IQANKTAKMLTKVPRTGTGLDRMIEKFLLKNLRQKPILTAYGFFALDKST
LFKLFTAIFTYMVILVQFKEMENSTKSINKF
Gr39D2c (SEQ ID NO: 32)
MKRNAFEELRVQLRTLKWLGVLRFTIDFNKCLVRENASEERSAWLYLIGV
VGITCSLIVYSTYFPSHFIMGKHNTTGNCYALINIRSCSIVTMLIYTQLY
IQRFRFVALLQSILRFNQISGSHREEGRFAFYYYTHLSLLIICMLNYAYG
YWTAGVRLTTIPIYLLQYGFSYLFLGQVVVLFACIQQILLSILKYYNQVV
LKNIKSSKESREFYYNFCKYNQVIWLSYTEINHCFGLLLLLVTGLILLIT
PSGPFYLVSTIFEGRFRQNWQFSLMSFTAILWSLPWIVLLVLAMGRNDVQ
KEANKTAKMLTKVPRTGTGLDRMIEKFLLKNLRQKPILTAYGFFALDKST
LFKLFTAIFTYMVILVQFKEMENSTKSINKF
Gr39D2d (SEQ ID NO: 33)
MSKVCRDLRIYLRLLHIMGMMCWHFDSDHCQLVATSGSERYAVVYAGCIL
VSTTAGFIFALLHPSRFHIAIYNQTGNFYEAVIFRSTCVVLFLVYVILYA
WRHRYRDLVQHILRLNRRCASSCTNQQFLHNIILYGMLTILCFGNYLHGY
TRAGLATLPLALCMLVYIFAFLVLCLLLMFFVSLKQVMTAGLIHYNQQLC
QGDLISGLRGRQQILKLCGGELNECFGLLMLPIVALVLLMAPSGPFFLIS
TVLEGKFRPDECLIMLLTSSTWDTPWMIMLVLMLRTNGISEEANKTAKML
TKVPRTGTGLDRMIEKFLLKNLRQKPILTAYGFFALDKSTLFKLFTAIFT
YMVILVQFKEMENSTKSINKF
Gr43C1 (SEQ ID NO: 34)
MKSATSKVVTALDVSVVVMAIVSGVYCGLFSLNDTLELNDRLNKIDNTLN
AYNNFRRDRWRALGMAAVSLLAISILVGLDVGTWMRIAQDMNIAQSDTEL
NVHWYIPFYSLYFILTGLQVNIANTAYGLGRRFGRLNRMLSSSFLAENNA
TSAIKPQKVSTVKNVSVNRPAMPSALHASLTKLNGETLPSEAAGDKAAAR
SLILNVELLKLGYFPAKNKGLLLKSLADSHESLGKCVHLLSNSFGIAVLF
ILVSCLLHLVATAYFLFLELLSKRDNGYLWVQMLWICFHFLRLLMVVEPC
HLAARESRKTIQIVCEIERKVHEPILAEAVKKFWQQLLVVDADFSACGLC
RVNRTILTSFASAIATYLVILIQFQRTNG
Gr47A1 (SEQ ID NO: 35)
MAFTSSQLCSLLTKFTALNGLNTYYFDTKTNAFRVSSKLKIYCAIHHALC
VLALAHMSYSTASNLRVSVTVLTIGGTMACCVKSCWEKAQGIRNLARGLV
TMEQKYFAGRPSGLLLKCRYYIKITFGSITLLRIHLIQPIYMRRLLPSQF
YLNVGAYWLLYNMLLAAVLGFYFLLWEMCRIQKLINDQMTLILARSGQRN
RLKKMQHCLRLYSKLLLLCDQFNSQLGHVAIWVLACKSWCQITFGYEIFQ
MVAAPKSIDLTMSMRVFVIFTYIFDAMNLFLGTDISELFSTFPADSQRIL
RETSRLDRLLSMFALKLALHPKRVVLLNVFTFDRKLTLTLLAKSTLYTIC
CLQNDYNKLKA
Gr58A1 (SEQ ID NO: 36)
MLLKFMYIYGIGCGLMPAPLKKGQFLLGYKQRWYLIYTACLHGGLLTVLP
FTFPHYMYDDSYMSSNPVLKWTFNLTNITRIMAMFSGVLLMWFRRKRILN
LGENLILHCLKCKTLDNRSKKYSKLRKRVRNVLFQMLLVANLSILLGALI
LFRIHSVQRISKTAMIVAHITQFIYVVFMMTGICVILLVLHWQSERLQIA
LKDLCSFLNHEERNSLTLSENKANRSLGKLAKLFKLFAENQRLVREVFRT
FDLPIALLLLKMFVTNVNLVYHCVQFGNDTIETSSYTRIVGQWVVISHYW
SAVLLMNVVDDVTRRSDLKMGDLLREFSHLELVKRDFHLQLELFSDHLRC
HPSTYKVCGLFIFNKQTSLAYFFYVLVQVLVLVQFDLKNKVEKRN
Gr58A2 (SEQ ID NO: 37)
MLHPKLGRVMNVVYYHSVVFALMSTTLRIRSCRKCLRLEKVSRTYTIYSF
FVGIFLFLNLYFMVPRIMEDGYMKYNIVLQWNFFVMLFLRAIAVVSCYGT
LWLKRHKIIQLYKYSLIYWKRFGHITRAIVDKKELLDLQESLARIMIRKI
ILLYSAFLCSTVLQYQLLSVINPQIFLAFCARLTHFLHFLCVKMGFFGVL
VLLNHQFLVIHLAINALHGRKARKKWKALRSVAAMHLKTLRLARRIFDMP
DIANATVFINMFMTAINILYHAVQYSNSSIKSNGWGILFGNGLIVFNFWG
TMALMEMLDSVVTSCNNTGQQLRQLSDLPKVGPKMQRELDYFTMQLRQNR
LVYKICGIVELDKPACLSYIGSILSNVIILMQFDLRRQRQPINDRQYLIH
LMKNKTKV
Gr58A3 (SEQ ID NO: 38)
MNQYFLLHTYFQVSRLIGLCNLHYDSSNHRFILNHVPTVVYCVILNVVYL
LVLPFALFVLTGNIYHCPDAGMFGVVYNVVALTKLLTMLFLMSSVWIQRR
RLYKLGNDLMKMLHKFRFNLGNDCRNRCLCKGLLTSSRFVLLTQQLLTRD
SVVNCESNSSLRQAMVPYQSAAIVYALIMILLMSYVDMTVYMVEVAGNWL
LVNMTQGVREMVQDLEVLPERNGIPREMGLMQILAAWRKLWRRCRRLDAL
LKQFVDIFQWQVLFNLLTTYIFSIAVLFRLWIYLEFDKNFHLWKGILYAI
IFLTHHVEIVMQFSIFEINRCKWLGLLEDVGNLWDINYSGRQCIKSSGTI
LSRKLEFSLLYMNRKLQLNPKRVRRLHIVGLFDISNLTVHNMTRSIITNV
LVLCQIAYKKYG
Gr59D1 (SEQ ID NO: 39)
MADLLKLCLRIAYAYGRLTGVINFKIDLKTGQALVTRGATLISVSTHLLI
FALLLYQTMRKSVVNVMWKYANSLHEYVFLVIAGFRVVCVFLELVSRWSQ
RRTFVRLFNSFRRLYQRNPDIIQYCRRSIVSKFFCVTMTETLHIIVTLAM
MRNRLSIALALRIWAVLSLTAIINVIITQYYVATACVRGRYALLNKDLQA
IVTESQSLVPNGGGVFVTKCCYLADRLERIAKSQSDLQELVENLSTAYEG
EVVCLVITYYLNMLGTSYLLFSISKYGNFGNNLLVIITLCGIVYFVFYVV
DCWINAFNVFYLLDAHDKMVKLLNKRTLFQPGLDHRLEMVFENFALNLVR
NPLKLHMYGLFEFGRGTSFAVFNSLLTHSLLLIQYDVQNF
Gr59D2 (SEQ ID NO: 40)
MVDLVKTILLIAYWYGLAVGVSNFEVDWLTGEAIATRRTTIYAAVHNASL
ITLLILFNLGNNSLKSEFISARYLHEYFFMLMTAVRISAVLLSLITRWYQ
RSRFIRIWNQILALVRDRPQVVRGRWYRRSIILKFVFCVLSDSLHTISDV
SAQRKRITADLIVKLSLLATLTTIFNMIVCQYYLAMVQVIGLYKILLQDL
RCLVRQAECICSIRNRRGGVYSIQCCSLADQLDLIAERHYFLKDRLDEMS
DLFQIQSLSMSLVYFFSTMGSIYFSVCSILYSSTGFGSTYWGLLLIVLST
ASFYMDNWLSVNIGFHIRDQQDELFRVLADRTLFYRELDNRLEAAFENFQ
LQLASNRHEFYVMGLPKMERGRLIAMLSSVITHTMVLVQWEIQN
Gr59E1 (SEQ ID NO: 41)
MRSSATKGAKLKNSPRERLSSFNPQYAERYKELYRTLFWLLLISVLANTA
PITILPGCPNRFYRLVRLSWMILWYGLFVLGSYWEFVLVTTQRVSLDRYL
NAIESAIYVVHIFSIMLLTWQCRNWAPKLMTNIVTSDLNRAYTIDCNRTK
RFIRLQLFLVGIFACLAIFFNIWTHKFVVYRSILSINSYVMPNIISISFA
QYYLLLQGIAWRQRRLTEGLERELTHLHSPRISEVQKIRMHHANLIDFTK
AVNRTFQYSILLLFVGCFLNFNLVLFLVYQGIENPSMADFTKWVCMLLWL
AMHVGKVCSILHFNQSIQNEHSTCLTLLSRVSYARKDIQDTITHFIIQMR
TNVRQHVVCGVINLDLKFLTTLLVASADFFIFLLQYDVTYEALSKSVQGN
VTRY
Gr59E2 (SEQ ID NO: 42)
MSDSSYWENLLLTINRFLGVYPSGRVGVLRWLHTLWSLFLLMYIWTGSIV
KCLEFTVEIPTIEKLLYLMEFPGNMATIAILVYYAVLNRPLAHGAELQIE
RIITGLKGKAKRLVYKRHGQRTLHLMATTLVFHGLCVLVDVVNYDFEFWT
TWSSNSVYNLPGLMMSLGVLQYAQPVHFLWLVMDQMRMCLKELKLLQRPP
QGSTKLDACYESAFAVLVDAGGGSALMIEEMRYTCNLIEQVHSQFLLRFG
LYLVLNLLNSLVSICVELYLIFNFFETPLWEESVLLVYRLLWLAMHGGRI
WFILSVNEQILEQKCNLCQLLNELEVCSSRLQRTINRFLLQLQRSIDQPL
EACGIVTLDTRSLGGFIGVLMAIVIFLIQIGLGNKSLMGVALNRSNWVYV
Gr6BD1 (SEQ ID NO: 43)
MKIYQDIYPISKPSQIFAILPFYSGDVDDGFRFGGLGRWYGRLVALIILI
GSLTLGEDVLFASKEYRLVASAQGDTEEINRTIETLLCIISYTMVVLSSV
QNASRHFRTLHDIAKIDEYLLANGFRETYSCRNLTILVTSAAGGVLAVAF
YYIHYRSGIGAKRQIILLLIYFLQLLYSTLLALYLRTLMMNLAQRIGFLN
QKLDTFNLQDCGHMENWRELSNLIEVLCKFRYITENINCVAGVSLLFYFG
FSFYTVTNQSYLAFATLTAGSLSSKTEVADTIGLSSIWVLAETITMIVIC
SACDGLASEVNGTAQILARIYGKSKQFQNLIDKFLTKSIKQDLQFTAYGF
FSIDNSTLFKIFSAVTTYLVILIQFKQLEDSKNLSRSYQLVM
Gr77E1 (SEQ ID NO:44)
MPRWLQLPGMSALGILYSLTRVFGLMATANWSPRGIKRVRQSLYLRIHGC
VMLIFVGCFSPFAFWCIFQRMAFLRQNRILLMIGFNRYVLLLVCAFMTLW
IHCFKQAEIIGCLNRLLKCRRRLRRLMHTRKLKDSMDCLATKGHLLEVVV
LLSSYLLSMAQPIQILKDDPEVRRNFMYACSLVFVSVCQAILQLSLGMYT
MAILFLGHLVRHSNLLLAKILADAEHIFESSQKAGFWPNRQELYKGQQKW
LALELWRLLHVHHQLLKLHRSICSLCAVQAVCFLGFVPLECTIHLFFTYF
MKYSKFILRKYGRSFPLNYFAIAFLVGLFTNLLLVILPTYYSERRFNCTR
EIIKGGGLAFPSRITVKQLRHTMHFYGLYLKNVEHVFAVSACGLFKLNNA
ILFCIVGAILEYLMILIQFDKVLN

b) Previously reported partial Gustatory Receptor sequences. Predicted proteins have been extended as disclosed in the subject application; extended sequence information is indicated in bold font.

Gr28A1 (SEQ ID NO: 45)
CQLLNGYRTEHAGGNYLLASDFDRRLKVFLQWKTSDSAESASGRLGSQYT
FVGHKKKQTGLTIKLAENGFCCWVLLLRYFSVLIKIVKYKIP
Gr57B1 (SEQ ID NO: 46)
MAVLYFFREPETVFDCAAFICILQFLMGCNGFGIRRSTFRISWASRIYSM
SVAIAAFCCLFGSLSVLLAEEDIRERLAKADNLVLSISALELLMSTLVFG
VTVISLQVFARRHLGIYQRLAALDARLMSDFGANLNYRKMLRKNIAVLGI
VTTIYLMAINSAAVQVASGHRALFLLFALCYTIVTGGPHFTGYHMTLAEM
LGIRFRLLQQLLQPEFLNWRFPQLHVQELRIRQVVSMIQELHYLIQEINR
VYALSLWAAMAHDLAMSTSELYILFGQSVGIGQQNEEENGSCYRMLGYLA
LVMIPPLYKLLIAPFYCDRTIYEARRCLRLVEKLDDWFPQKSSLRPLVES
LMSWRIQAKIQFTSGLDVVLSRKVIGLFTSILVNYLLILIQFAMTQKMGE
QIEQQKIALQEWIGF
Gr65C1 (SEQ ID NO: 47)
MRVHQRQSAVIIQMGHPPFMSLKGGKSGFGSIVWPSAMREVNLLNRFTRQ
FLFLIVLVTQICGVATFVYNSKAQCFRQSGFLRFYSSLVLIFLALFLIVT
TSKMFHNLQAVWPYVVGSVIILVVRIHGLLESAEIVELLNQMLRIMRQVN
LMARHPNLFRLKHLLLLLLALQNLLRSLNTIVGISNHSAEAYDSFLNSVI
LLIILAVLLSFLLQITINICLFVVLIATYSELHHCTRRISNDMDKLRLHS
VHESGQFMVLVKQLQGITEKLIRLRQNVFHITVRITRHFRFHWLCAIIYG
LLPFFSLTAKDQNGFNFLIISALNIIFQWTIFAILSRES
Gr93F1 (SEQ ID NO: 48)
MTGKRAESWSRLLLLWLYRCARGLLVLSSSLDRDKLQLKATKQGSRNRFL
HILWRCIVVMIYAGLWPMLTSAVIGKRLESYADVLALAQSMSVSILAVIS
FVIQARGENQFREVLNRYLALYQRICLTTRLRHLFPTKFVVFFLLKLFFT
LCGCFHEIIPLFENSHFDDISQMVGTGFGIYMWLGTLCVLDACFLGFLVS
GILYEHMANNIIAMLKRMEPIESQDERYRMTKYRRMQLLCDFADELDECA
AIYSELYHVTNSFRRILQWQILFYIYLNFINICLMLYQYILHFLNDDEVV
FVSIVMAFVICLANLVLLMHCADYTVRQSEVPKKLPLDIVCSDMDERWDK
SVSLLLFETFLGQLQTQRLEIKVLGFFHLNNEFILLILSAIISYLFILIQ
FGITGGFEASEDIKNFAD
Gr93F2 (SEQ ID NO: 49)
MQFWFGEELINLVNRFLQLFRRMQSLTNSPKNRFGDRAEFLLMFSKVFSL
LFVFMAFRLMLSPWFLLTLVCDLYTSVGTGMITHLCFVGYLSIGVLYRDL
NNYVDCQLRAQLRSLNGENNSFRNNPQPTRQAISNLDKCLYLYDEIHQVS
RSFQQLFDLPLFLSLAQSLLAMSMVSYHAILRRQYSFNLWGLVIKLLIDV
VLLTMSVHSAVNGSRLIRRLSFENFYVTDSQSYHQKVSPGAIILRIKYNT
FPILQLELFLGRLQHQELRVFPLGLFEVSNELTLFFLSAMVTYLVFLVQ
Gr93F3 (SEQ ID NO: 50)
MIERLKKVSLPALSAFILFCSCHYGRILGVICFDIGQRTSDDSLVVRNRH
QFKWFCLSCRLISVTAVCCFCAPYVADIEDPYERLLQCFRLSASLICGIC
IIVVQVCYEKELLRMIISFLRLFRRVRRLSSLKRIGFGGKREFFLLLFKF
ICLVYELYSEICQLWHLPDSLSLFATLCEIFLEIGSLMIIHIGFVGYLSV
AALYSEVNSFARIELRRQLRSLERPVGGPVGRKQLRIVEYRVDECISVYD
EIERVGRTFHRLLELPVLIILLGKIFATTILSYEVIIRPELYARKIGMWG
LVVKSFADVILLTLAVHEAVSSSRMMRRLSLENFPITDHKAWHMKVSDIM
VFLIKCIFFSRLQWEMFLSRLNFFEFRVRPLGLFEVSNEVILLFLSSMIT
YFTYVVQ
Gr93F4 (SEQ ID NO: 51)
MSFYARFLSLVCFRLRKQKDNNVWLEEIWSNRSRWKWISVTLRIVPLCIY
AFTYAEWISNRMLITEKFLHSCSLVVSIPCYLSIIHLKICHGPEVTKLVN
QYLHIFRLGTLDIRRRSQFGGGRELFLLILSVCCQIHEYVFILVIASRLC
GFQHIIWWVSYTYVFIICNSIMCFGFIWHLSLGVLYAELNDNLRFESGFQ
TAPLRKQQRIRVQKSMALFKEISSVVTSLQDIFNVHLFLSALLTLLQVLV
VWYKMIIDLGFSDFRIWSFSLKNLIQTLLPVLAIQEAANQFKQTREPALD
IFLVGKSKHWMKSVSKLINQGILQLIGLFNVSNELFLIIVSAMFCYLVFV
TQCVIVYRRRYVI
Gr94E1 (SEQ ID NO: 52)
MDFTSDYAHRRMVKFLTIILIGFMTVFGLLANRYRAGRRERFRFSKANLA
FASLWAIAFSLVYGRQIYKEYQEGQINLKDATTLYSYIWITVAVINYVSQ
MIISDHVAKVLSKVPFFDTLKEFRLDSRSLYISIVLALVKTVAFPLTIEV
AFILQQRRQHPEMSLIWTLYRLFPLIISNFLNNCYFGAMVVVKEILYALN
RRLEAQLQEVNLLQRKDQLKLYTKYYRMQRFCALADELDQLAYRYRLIYV
HSGKYLTPMSLSMILSLICHLLGITVGFYSLYYAIADTLIMGKPYDGLGS
LINLVFLSISLAEITLLTHLCNHLLVATRRSAVILQEMNLQHADSRYRQA
VHGFTLLVTVTKYQIKPLGLYELDMRLISNVFSAVASFLLILVQADLSQR
FKMQ
Gr97D1 (SEQ ID NO: 53)
MRFLRRQTRRLRSIWQRSLPVRFRRGKLHTQLVTICLYATVFLNILYGVY
LGRFSFRRKKFVFSKGLTIYSLFVATFFALFYIWNIYNEISTGQINLRDT
IGIYCYMNVCVCLFNYVTQWEKTLQIIRFQNSVPLFKVLDSLDISAMIVW
RAFIYGLLKIVFCPLITYITLILYHRRSISESQWTSVTTTKTMLPLIVSN
QINNCFFGGLVLIFAAVNRKLHGIVKEANMLQSPVQMNLHKPYYRMRRFC
ELADLLDELARKYFGTASRSKNYLRFTDWSMVLSMLMNLLGITMGCYNQY
LAIADHYINEEPFDLFLAIVLVVFLAVPFLELVMVARISNQTLVEVIVI
Gr98B1 (SEQ ID NO: 54)
IERFVCAQLVHEAYKQFASNGFRFLDALGCYEHSALGRARPLSRRGYAIK
VSDHPATPPHYHMPPPKQPPSHLAVQHATLTSGLRQLSFSCVNCNCSRCC
WSLPMHFRYIFNASLCNCQRQ*GY*TLSCRRHCTATKNISFSFCHISFVF
LLKYDPKNPQLR
GrLU1 = Gr36B1 (SEQ ID NO: 55)
MFDWVGLLLKVLYYYGQIIGLINFEIDWQRGRVVAAQRGILFAIAINVLI
CMVLLLQISKKFNLDVYFGRANQLHQYVIIVMVSLRMASLNRWRQRAQLM
RLVECVLRLFLKKPHVKQMSRWAILVKFSVGVVSNFLQMAISMESLDRLG
FNEFVGMASDFWMSAIINMAISQHYLVILFVRAYYHLLKTEVRQAIHESQ
MLSEIYPRRAAFMTKCCYLADRIDNIAKLQNQLQSIVTQLNQVFGIQGIM
VYGGYYIFSVATTYITYSLAINGIEELHLSVRAAALVSWFLFYYTSAILN
LFVMLKLFDDHKEMERILEERTLFTSALDVRLEQSVSFYPTITELKYRDL
VLSQFESIQLQLIRNPLKIEVLDIFTITRSSSAAMIGSIITNSIFLIQYD
MEYF
GrLU2 = Gr28A3 (SEQ ID NO: 56)
MWLLRRSVGKSGNRPHDVYTCYRLTIFMALCLGIVPYYVSISSEGRGKLT
SSYIGYINIIIRMAIYMVNSFYGAVNRDTLMSNFFLTDISNVIDALQKIN
GMLGIFAILLISLLNRKELLKLLATFDRLETEAFPRVLKNLAHQWDTRSL
KAVNQKQRSLQCLDSFSMYTIVTKDPAEIIQESMEIHHLICEAAATANKY
FTYQLLTIISIAFLIIVFDAYYVLETLLGKSKRESKFKTVEFVTFFSCQM
ILYLIAIISIVEGSNRAIKKSEKTGGIVHSLLNKTKSAEVKEKLQQFSMQ
LMHLKINFTAAGLFNIDRTLYFTISGALTTYLIILLQFTSNSPNNGYGNG
SSCCETFNNMTNHTL
GrLU3 = Gr64A1 (SEQ ID NO: 57)
MKGPNLNFRKTPSKDNGVKQVESLARPETPPPKFVEDSNLEFNVLASEKL
PNYTNLDLFHRAVFPFMFLAQCVAIMPLVGIRESNPRRVRFAYKSIPMFV
TLIFMIATSILFLSMFTHLLKIGITAKNFVGLVFFGCVLSAYVVFIRLAK
KWPAVVRIWTRTEIPFTKPPYEIPKRNLSRRVQLAALAIIGLSLGEHALY
QVSAILSYTRRIQHCANITTVPSFNNYMQTNYDYVFQLLPYSPIIAVLIL
ATCTFVWNYNDLFIMMISKGLSYRFEQITTRIRKLEHEEVCESVFIQIRE
HYVKMCELLEFVDSAMSSLILLSCVNNLYFVCYQLLNVFNKLRWPINYIY
IYFWYSLLYLIGRTAFVFLTAADINEESKRGLGVLRRVSSRSWCVEVERL
IFQMTTQTVALSGKKFYFLTRRLLFGMAGTIVTYELVLLQFDEPNRRKGL
QP
GrLU4 (SEQ ID NO: 58)
IYILSLYIFFQFISNVSLIVVLKLFRDI
GrLU7 = Gr5A1 (SEQ ID NO: 59)
MRQLKGRNRCNRAVRHLKVQGKMWLKNLKSGLEQIRESQVRGTRKNFLHD
GSFHEAVAPVLAVAQCFCLMPVCGISAPTYRGLSFNRRSWRFWYSSLYLC
STSVDLAFSIRRVAHSVLDVRSVEPIVFHVSILIASWQFLNLAQLWPGLM
RHWAAVERRLPGYTCCLQRARPARRLKLVAFVLLVVSLMEHLLSIISVVY
YDFCPRRSDPVESYLLGASAQLFEVFPYSNWLAWLGKIQNVLLTFGWSYM
DIFLMMLGMGLSEMLARLMRSLEQQVRQPMPEAYWTWSRTLYRSIVELIR
EVDDAVSGIMLISFGSNLYFICLQLLKSINTMPSSAHAVYFYFSLLFLLS
RSTAVLLFVSAINDQAREPLRLLRLVPLKGYHPEVFRFAAELASDQVALT
GLKFFNVTRKLFLAMAGTVATYELVLIQFHEDKKTWDCSPFNLD

The family of receptors disclosed herein has a signature motif which comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

The invention provides an isolated nucleic acid encoding an insect gustatory receptor protein, wherein the receptor protein comprises seven transmembrane domains and a C-terminal domain, and the C-terminal domain comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

The invention provides an isolated nucleic acid encoding an insect odorant receptor protein, wherein the receptor protein comprises seven transmembrane domains and a C-terminal domain, and the C-terminal domain comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

The invention provides an isolated nucleic acid molecule encoding an insect gustatory receptor protein, wherein the nucleic acid molecule encodes a protein selected from the group consisting of:
(a) an insect gustatory receptor protein comprising consecutive amino acids having the sequence of any of the receptors disclosed herein;
(b) an insect gustatory receptor protein which shares from 7-50% amino acid identity with any one of the proteins of (a), and comprises seven transmembrane domains and a C-terminal domain, wherein the C-terminal domain comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

The invention provides an isolated nucleic acid molecule encoding an insect odorant receptor protein, wherein the nucleic acid molecule encodes a protein selected from the group consisting of:
(a) an insect odorant receptor protein comprising consecutive amino acids having the sequence of any of the receptors disclosed herein;
(b) an insect odorant receptor protein which shares from 7-50% amino acid identity with any one of the proteins of (a), and comprises seven transmembrane domains and a C-terminal domain, wherein the C-terminal domain comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

The invention provides an isolated nucleic acid encoding an insect gustatory receptor protein, wherein the nucleic acid molecule encodes a protein selected from the group consisting of:
(a) an insect receptor protein comprising consecutive amino acids having a sequence identical to that set forth for Gr2B1 in SEQ ID NO: 1,
(b) an insect receptor protein comprising consecutive amino acids having a sequence identical to that set forth for Gr8D1 in SEQ ID NO: 2,
(c) an insect receptor protein comprising consecutive amino acids having a sequence identical to that set forth for Gr10B1 in SEQ ID NO: 3,
(d) an insect receptor protein comprising consecutive amino acids having a sequence identical to that set forth for Gr10B2 in SEQ ID NO: 4,
(e) an insect receptor protein comprising consecutive amino acids having a sequence identical to that set forth for Gr28A2 in SEQ ID NO: 5,
(f) an insect receptor protein comprising consecutive amino acids having a sequence identical to that set forth for Gr28A4 in SEQ ID NO: 6,
(g) an insect receptor protein comprising consecutive amino acids having a sequence identical to that set forth for Gr33C1 in SEQ ID NO: 7,
(h) an insect receptor protein comprising consecutive amino acids having a sequence identical to that set forth for Gr36B2 in SEQ ID NO: 8,
(i) an insect receptor protein comprising consecutive amino acids having a sequence identical to that set forth for Gr36B3 in SEQ ID NO: 9,
(j) an insect receptor protein comprising consecutive amino acids having a sequence identical to that set forth for Gr59C1 in SEQ ID NO: 10,
(k) an insect receptor protein comprising consecutive amino acids having a sequence identical to that set forth for Gr61D1 in SEQ ID NO: 11,
(l) an insect receptor protein comprising consecutive amino acids having a sequence identical to that set forth for Gr63F1 in SEQ ID NO: 12,
(m) an insect receptor protein comprising consecutive amino acids having a sequence identical to that set forth for Gr64A2 in SEQ ID NO: 13,
(n) an insect receptor protein comprising consecutive amino acids having a sequence identical to that set forth for GR64A3 in SEQ ID NO: 14,
(o) an insect receptor protein comprising consecutive amino acids having a sequence identical to that set forth for Gr66C1 in SEQ ID NO: 15,
(p) an insect receptor protein comprising consecutive amino acids having a sequence identical to that set forth for Gr92D1 in SEQ ID NO: 16,
(q) an insect receptor protein comprising consecutive amino acids having a sequence identical to that set forth for Gr98A1 in SEQ ID NO: 17,
(r) an insect receptor protein comprising consecutive amino acids having a sequence identical to that set forth for Gr98A2 in SEQ ID NO: 18,
(s) an insect receptor protein comprising consecutive amino acids having a sequence identical to that set forth for Gr2940.1 in SEQ ID NO: 19,
(t) an insect receptor protein comprising consecutive amino acids having a sequence identical to that set forth for Gr2940.2 in SEQ ID NO: 20,
(u) an insect receptor protein comprising consecutive amino acids having a sequence identical to that set forth for Gr2940.3 in SEQ ID NO: 21,
(v) an insect receptor protein comprising consecutive amino acids having a sequence identical to that set forth for Gr2940.4 in SEQ ID NO: 22,
(w) an insect receptor protein comprising consecutive amino acids having a sequence identical to that set forth for Gr2940.5 in SEQ ID NO: 23,
(x) an insect receptor protein comprising consecutive amino acids having a sequence identical to that set forth for Gr57B1 in SEQ ID NO: 46,
(y) an insect receptor protein comprising consecutive amino acids having a sequence identical to that set forth for Gr93F1 in SEQ ID NO: 48,
(z) an insect receptor protein comprising consecutive amino acids having a sequence identical to that set forth for Gr93F2 in SEQ ID NO: 49,
(aa) an insect receptor protein comprising consecutive amino acids having a sequence identical to that set forth for Gr93F3 in SEQ ID NO: 50,
(bb) an insect receptor protein comprising consecutive amino acids having a sequence identical to that set forth for Gr93F4 in SEQ ID NO: 51,
(cc) an insect receptor protein comprising consecutive amino acids having a sequence identical to that set forth for Gr94E1 in SEQ ID NO: 52,
(dd) an insect receptor protein comprising consecutive amino acids having a sequence identical to that set forth for Gr93D1 in SEQ ID NO: 53,
(ee) an insect receptor protein comprising consecutive amino acids having a sequence identical to that set forth for GrLU1=Gr36B1 in SEQ ID NO: 55,
(ff) an insect receptor protein comprising consecutive amino acids having a sequence identical to that set forth for GrLU2=Gr28A3 in SEQ ID NO: 56,
(gg) an insect receptor protein comprising consecutive amino acids having a sequence identical to that set forth for GrLU3=Gr64A1 in SEQ ID NO: 57,
(hh) an insect receptor protein comprising consecutive amino acids having a sequence identical to that set forth for GrLU7=Gr5A1 in SEQ ID NO: 59, and
(ii) an insect gustatory receptor protein which shares from 7-50% amino acid identity with any one of the proteins of (a)-(hh), and comprises seven transmembrane domains and a C-terminal domain, wherein the C-terminal domain comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

In one embodiment, the insect odorant receptor protein shares at least 20% amino acid identity with any one of the proteins described herein. In one embodiment, the insect odorant receptor protein shares at least 30% amino acid identity with any one of the proteins described herein. In one embodiment, the insect odorant receptor protein shares at least 40% amino acid identity with any one of the proteins described herein. In one embodiment, the insect odorant receptor protein shares at least 50% amino acid identity with any one of the proteins described herein. In one embodiment, the insect odorant receptor protein shares at least 60% amino acid identity with any one of the proteins described herein. In one embodiment, the insect odorant receptor protein shares at least 70% amino acid identity with any one of the proteins described herein. In one embodiment, the insect odorant receptor protein shares at least 80% amino acid identity with any one of the proteins described herein.

The invention provides an isolated nucleic acid molecule encoding an insect odorant receptor protein, wherein the nucleic acid molecule encodes a protein selected from the group consisting of:
(a) an insect receptor protein comprising consecutive amino acids having a sequence identical to that set forth for Gr2B1 in SEQ ID NO: 1,
(b) an insect receptor protein comprising consecutive amino acids having a sequence identical to that set forth for Gr8D1 in SEQ ID NO: 2,
(c) an insect receptor protein comprising consecutive amino acids having a sequence identical to that set forth for Gr10B1 in SEQ ID NO: 3,
(d) an insect receptor protein comprising consecutive amino acids having a sequence identical to that set forth for Gr10B2 in SEQ ID NO: 4,
(e) an insect receptor protein comprising consecutive amino acids having a sequence identical to that set forth for Gr28A2 in SEQ ID NO: 5,
(f) an insect receptor protein comprising consecutive amino acids having a sequence identical to that set forth for Gr28A4 in SEQ ID NO: 6,
(g) an insect receptor protein comprising consecutive amino acids having a sequence identical to that set forth for Gr33C1 in SEQ ID NO: 7,
(h) an insect receptor protein comprising consecutive amino acids having a sequence identical to that set forth for Gr36B2 in SEQ ID NO: 8,
(i) an insect receptor protein comprising consecutive amino acids having a sequence identical to that set forth for Gr36B3 in SEQ ID NO: 9,
(j) an insect receptor protein comprising consecutive amino acids having a sequence identical to that set forth for Gr59C1 in SEQ ID NO: 10,
(k) an insect receptor protein comprising consecutive amino acids having a sequence identical to that set forth for Gr61D1 in SEQ ID NO: 11,
(l) an insect receptor protein comprising consecutive amino acids having a sequence identical to that set forth for Gr63F1 in SEQ ID NO: 12,
(m) an insect receptor protein comprising consecutive amino acids having a sequence identical to that set forth for Gr64A2 in SEQ ID NO: 13,
(n) an insect receptor protein comprising consecutive amino acids having a sequence identical to that set forth for GR64A3 in SEQ ID NO: 14,
(o) an insect receptor protein comprising consecutive amino acids having a sequence identical to that set forth for Gr66C1 in SEQ ID NO: 15,
(p) an insect receptor protein comprising consecutive amino acids having a sequence identical to that set forth for Gr92D1 in SEQ ID NO: 16,
(q) an insect receptor protein comprising consecutive amino acids having a sequence identical to that set forth for Gr98A1 in SEQ ID NO: 17,
(r) an insect receptor protein comprising consecutive amino acids having a sequence identical to that set forth for Gr98A2 in SEQ ID NO: 18,
(s) an insect receptor protein comprising consecutive amino acids having a sequence identical to that set forth for Gr2940.1 in SEQ ID NO: 19,
(t) an insect receptor protein comprising consecutive amino acids having a sequence identical to that set forth for Gr2940.2 in SEQ ID NO: 20,
(u) an insect receptor protein comprising consecutive amino acids having a sequence identical to that set forth for Gr2940.3 in SEQ ID NO: 21,
(v) an insect receptor protein comprising consecutive amino acids having a sequence identical to that set forth for Gr2940.4 in SEQ ID NO: 22,
(w) an insect receptor protein comprising consecutive amino acids having a sequence identical to that set forth for Gr2940.5 in SEQ ID NO: 23,
(x) an insect receptor protein comprising consecutive amino acids having a sequence identical to that set forth for Gr57B1 in SEQ ID NO: 46,
(y) an insect receptor protein comprising consecutive amino acids having a sequence identical to that set forth for Gr93F1 in SEQ ID NO: 48,
(z) an insect receptor protein comprising consecutive amino acids having a sequence identical to that set forth for Gr93F2 in SEQ ID NO: 49,
(aa) an insect receptor protein comprising consecutive amino acids having a sequence identical to that set forth for Gr93F3 in SEQ ID NO: 50,
(bb) an insect receptor protein comprising consecutive amino acids having a sequence identical to that set forth for Gr93F4 in SEQ ID NO: 51,
(cc) an insect receptor protein comprising consecutive amino acids having a sequence identical to that set forth for Gr94E1 in SEQ ID NO: 52,
(dd) an insect receptor protein comprising consecutive amino acids having a sequence identical to that set forth for Gr93D1 in SEQ ID NO: 53,
(ee) an insect receptor protein comprising consecutive amino acids having a sequence identical to that set forth for GrLU1=Gr36B1 in SEQ ID NO: 55,
(ff) an insect receptor protein comprising consecutive amino acids having a sequence identical to that set forth for GrLU2=Gr28A3 in SEQ ID NO: 56,
(gg) an insect receptor protein comprising consecutive amino acids having a sequence identical to that set forth for GrLU3=Gr64A1 in SEQ ID NO: 57,
(hh) an insect receptor protein comprising consecutive amino acids having a sequence identical to that set forth for GrLU7=Gr5A1 in SEQ ID NO: 59, and
(ii) an insect odorant receptor protein which shares from 7-50% amino acid identity with any one of the proteins of (a)-(hh), and comprises seven transmembrane domains and a C-terminal domain, wherein the C-terminal domain comprises consecutive amino acids having the following sequence:

(SEQ ID NO: 60)

-G-L/F-F-X-X-X-X-X-X-X-X-X-X-X-X-X-X-T-Y-L-V/I-L-
V/I/L-Q-F-,

where X is any amino acid, and / means or.

In one embodiment, the insect gustatory receptor protein shares at least 20% amino acid identity with any one of the proteins described herein. In one embodiment, the insect gustatory receptor protein shares at least 30% amino acid identity with any one of the proteins described herein. In one embodiment, the insect gustatory receptor protein shares at least 40% amino acid identity with any one of the proteins described herein. In one embodiment, the insect gustatory receptor protein shares at least 50% amino acid identity with any one of the proteins described herein. In one embodiment, the insect gustatory receptor protein shares at least 60% amino acid identity with any one of the proteins described herein. In one embodiment, the insect gustatory receptor protein shares at least 70% amino acid identity with any one of the proteins described herein. In one embodiment, the insect gustatory receptor protein shares at least 80% amino acid identity with any one of the proteins described herein.

In one embodiment of any of the isolated nucleic acid molecules described herein, the insect gustatory or odorant receptor protein comprises seven transmembrane domains.

In different embodiments of any of the isolated nucleic acid molecules described herein, the nucleic acid is DNA or RNA. In different embodiments, the DNA is cDNA, genomic DNA, or synthetic DNA.

In one embodiment of any of the isolated nucleic acid molecules described herein, the nucleic acid molecule encodes a Drosophila receptor.

The nucleic acid molecules encoding an insect gustatory or odorant receptor include molecules coding for polypeptide analogs, fragments or derivatives of antigenic polypeptides which differ from naturally-occurring forms in terms of the identity or location of one or more amino acid residues (deletion analogs containing less than all of the residues specified for the protein, substitution analogs wherein one or more residues specified are replaced by other residues and addition analogs where in one or more amino acid residues is added to a terminal or medial portion of the polypeptides) and which share some or all properties of naturally-occurring forms.

These molecules include but not limited to: the incorporation of codons “preferred” for expression by selected non-mammalian hosts; the provision of sites for cleavage by restriction endonuclease enzymes; and the provision of additional initial, terminal or intermediate sequences that facilitate construction of readily expressed vectors. Accordingly, these charges may result in a modified insect receptor. It is the intent of this invention to include nucleic acid molecules which encode modified insect receptors. Also, to facilitate the expression of receptors in different host cells, it may be necessary to modify the molecule such that the expressed receptors may reach the surface of the host cells. The modified insect receptor should have biological activities similar to the unmodified insect gustatory or odorant receptor. The molecules may also be modified to increase the biological activity of the expressed receptor.

The invention provides a nucleic acid molecule comprising at least 12 nucleotides which specifically hybridizes with any of the isolated nucleic acid molecules described herein.

In one embodiment, the nucleic acid molecule hybridizes with a unique sequence within the sequence of any of the nucleic acid molecules described herein. In different embodiments, the nucleic acid is DNA, cDNA, genomic DNA, synthetic DNA, RNA, or synthetic RNA.

This invention provides a vector which comprises any of the isolated nucleic acid molecules described herein. In one embodiment, the vector is a plasmid.

In one embodiment of any of the vectors described herein, any of the isolated nucleic acid molecules described herein is operatively linked to a regulatory element.

Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG. Similarly, a eukaryotic expression vector includes a heterologous or homologous promoter for RNA polymerase II, a downstream polyadenylation signal, the start codon AUG, and a termination codon for detachment of the ribosome. Such vectors may be obtained commercially or assembled from the sequences described by methods well-known in the art, for example the methods described herein for constructing vectors in general.

The invention provides a host vector system for production of a polypeptide having the biological activity of an insect gustatory or odorant receptor, which comprises any of the vectors described herein and a suitable host. In different embodiments, the suitable host is a bacterial cell, a yeast cell, an insect cell, or an animal cell.

The host cell of the expression system described herein may be selected from the group consisting of the cells where the protein of interest is normally expressed, or foreign cells such as bacterial cells (such as E. coli), yeast cells, fungal cells, insect cells, nematode cells, plant or animal cells, where the protein of interest is not normally expressed. Suitable animal cells include, but are not limited to Vero cells, HeLa cells, Cos cells, CV1 cells and various primary mammalian cells.

The invention provides a method of producing a polypeptide having the biological activity of an insect gustatory or odorant receptor which comprising growing any of the host vector systems described herein under conditions permitting production of the polypeptide and recovering the polypeptide so produced.

The invention provides a purified insect gustatory or odorant receptor protein encoded by any of the isolated nucleic acid molecules described herein. This invention further provides a polypeptide encoded by any of the isolated nucleic acid molecules described herein.

The invention provides an antibody which specifically binds to an insect gustatory or odorant receptor protein encoded by any of the isolated nucleic acid molecules described herein. In one embodiment, the antibody is a monoclonal antibody. In another embodiment, the antibody is polyclonal.

The invention provides an antibody which competitively inhibits the binding of any of the antibodies described herein capable of specifically binding to an insect gustatory or odorant receptor. In one embodiment, the antibody is a monoclonal antibody. In another embodiment, the antibody is polyclonal.

Monoclonal antibody directed to an insect gustatory or odorant receptor may comprise, for example, a monoclonal antibody directed to an epitope of an insect gustatory or odorant receptor present on the surface of a cell. Amino acid sequences may be analyzed by methods well known to those skilled in the art to determine whether they produce hydrophobic or hydrophilic regions in the proteins which they build. In the case of cell membrane proteins, hydrophobic regions are well known to form the part of the protein that is inserted into the lipid bilayer which forms the cell membrane, while hydrophilic regions are located on the cell surface, in an aqueous environment.

Antibodies directed to an insect gustatory or odorant receptor may be serum-derived or monoclonal and are prepared using methods well known in the art. For example, monoclonal antibodies are prepared using hybridoma technology by fusing antibody producing B cells from immunized animals with myeloma cells and selecting the resulting hybridoma cell line producing the desired antibody. Cells such as NIH3T3 cells or 293 cells which express the receptor may be used as immunogens to raise such an antibody. Alternatively, synthetic peptides may be prepared using commercially available machines.

As a still further alternative, DNA, such as a cDNA or a fragment thereof, encoding the receptor or a portion of the receptor may be cloned and expressed. The expressed polypeptide may be recovered and used as an immunogen.

The resulting antibodies are useful to detect the presence of insect gustatory or odorant receptors or to inhibit the function of the receptor in living animals, in humans, or in biological tissues or fluids isolated from animals or humans.

This antibodies may also be useful for identifying or isolating other insect gustatory or odorant receptors. For example, antibodies against the Drosophila odorant receptor may be used to screen an cockroach expression library for a cockroach gustatory or odorant receptor. Such antibodies may be monoclonal or monospecific polyclonal antibody against a selected insect gustatory or odorant receptor. Different insect expression libraries are readily available and may be made using technologies well-known in the art.

One means of isolating a nucleic acid molecule which encodes an insect gustatory or odorant receptor is to probe a libraries with a natural or artificially designed probes, using methods well known in the art. The probes may be DNA, cDNA or RNA. The library may be cDNA or genomic DNA.

The invention provides a method of transforming a cell which comprises transfecting a host cell with any of the vectors described herein.

The invention provides a transformed cell produced by any of the methods described herein. In one embodiment, prior to being transfected with the vector the host cell does not express a gustatory or an odorant receptor protein. In one embodiment, prior to being transfected with the vector the host cell does not express a gustatory and an odorant receptor protein. In one embodiment, prior to being transfected with the vector the host cell does express a gustatory or odorant receptor protein.

This invention provides a method of identifying a compound which specifically binds to an insect gustatory receptor which comprises contacting any of the transformed cells described herein, or a membrane fraction from said cells, with the compound under conditions permitting binding of the compound to the gustatory receptor, detecting the presence of any such compound specifically bound to the receptor, and thereby identifying the compound as a compound which specifically binds to an insect gustatory receptor.

This invention provides a method of identifying a compound which specifically binds to an insect odorant receptor which comprises contacting any of the transformed cells described herein, or a membrane fraction from said cells, with the compound under conditions permitting binding of the compound to the odorant receptor, detecting the presence of any such compound specifically bound to the receptor, and thereby identifying the compound as a compound which specifically binds to an insect odorant receptor.

This invention provides a method of identifying a compound which specifically binds to an insect gustatory receptor which comprises contacting any of the purified insect gustatory receptor proteins described herein with the compound under conditions permitting binding of the compound to the purified gustatory receptor protein, detecting the presence of any such compound specifically bound to the receptor, and thereby identifying the compound as a compound which specifically binds to an insect gustatory receptor.

This invention provides a method of identifying a compound which specifically binds to an insect odorant receptor which comprises contacting any of the purified insect odorant receptor proteins described herein with the compound under conditions permitting binding of the compound to the purified odorant receptor protein, detecting the presence of any such compound specifically bound to the receptor, and thereby identifying the compound as a compound which specifically binds to an insect odorant receptor.

In one embodiment, the purified insect gustatory or odorant receptor protein is embedded in a lipid bilayer. The purified receptor may be embedded in the liposomes with proper orientation to carry out normal functions. Liposome technology is well-known in the art.

The invention provides a method of identifying a compound which activates an insect gustatory receptor which comprises contacting any of the transformed cells described herein, or a membrane fraction from said cells, with the compound under conditions permitting activation of the gustatory receptor, detecting activation of the receptor, and thereby identifying the compound as a compound which activates an insect gustatory receptor.

The invention provides a method of identifying a compound which activates an insect odorant receptor which comprises contacting any of the transformed cells described herein, or a membrane fraction from said cells, with the compound under conditions permitting activation of the odorant receptor, detecting activation of the receptor, and thereby identifying the compound as a compound which activates an insect odorant receptor.

The invention provides a method of identifying a compound which activates an insect gustatory receptor which comprises contacting any of the purified insect gustatory receptor proteins described herein with the compound under conditions permitting activation of the gustatory receptor, detecting activation of the receptor, and thereby identify the compound as a compound which activates an insect gustatory receptor.

The invention provides a method of identifying a compound which activates an insect odorant receptor which comprises contacting any of the purified insect odorant receptor proteins described herein with the compound under conditions permitting activation of the odorant receptor, detecting activation of the receptor, and thereby identify the compound as a compound which activates an insect odorant receptor.

In one embodiment, the purified insect gustatory or odorant receptor protein is embedded in a lipid bilayer. The purified receptor may be embedded in the liposomes with proper orientation to carry out normal functions. Liposome technology is well-known in the art.

The invention provides a method of identifying a compound which inhibits the activity of an insect gustatory receptor which comprises contacting any of the transformed cells described herein, or a membrane fraction from said cells, with the compound under conditions permitting inhibition of the activity of the gustatory receptor, detecting inhibition of the activity of the receptor, and thereby identifying the compound as a compound which inhibits the activity of an insect gustatory receptor.

The invention provides a method of identifying a compound which inhibits the activity of an insect odorant receptor which comprises contacting any of the transformed cells described herein, or a membrane fraction from said cells, with the compound under conditions permitting inhibition of the activity of the odorant receptor, detecting inhibition of the activity of the receptor, and thereby identifying the compound as a compound which inhibits the activity of an insect odorant receptor.

The invention provides a method of identifying a compound which inhibits the activity of an insect gustatory receptor which comprises contacting any of the purified insect gustatory receptor proteins described herein with the compound under conditions permitting inhibition of the activity of the gustatory receptor, detecting inhibition of the activity of the receptor, and thereby identifying the compound as a compound which inhibits the activity of an insect gustatory receptor.

The invention provides a method of identifying a compound which inhibits the activity of an insect odorant receptor which comprises contacting any of the purified insect odorant receptor proteins described herein with the compound under conditions permitting inhibition of the activity of the odorant receptor, detecting inhibition of the activity of the receptor, and thereby identifying the compound as a compound which inhibits the activity of an insect odorant receptor.

In one embodiment, the purified insect gustatory or odorant receptor protein is embedded in a lipid bilayer. The purified receptor may be embedded in the liposomes with proper orientation to carry out normal functions. Liposome technology is well-known in the art.

In one embodiment of any of the methods described herein, the compound is not previously known.

The invention provides a compound identified by any of the methods described herein. In one embodiment, the compound is an alarm odorant ligand or a ligand associated with fertility. In one embodiment the compound interferes with chemosensory perception.

The invention provides a method of combating ingestion of crops by pest insects which comprises identifying a compound by any of the methods described herein and spraying the crops with the compound.

The invention provides a use of a compound identified by any of the methods described herein for combating ingestion of crops by pest insects.

The invention provides a use of a compound identified by any of the methods described herein for combating pest nuisances and disease-carrying insects by interfering with chemosensory perception.

The invention provides a method of combating disease-carrying insects in an area which comprises identifying a compound by any of the methods described herein and spraying the area with the compound.

The invention provides a method of controlling a pest population in an area which comprises identifying a compound any of the methods described herein and spraying the area with the compound. In one embodiment, the compound is an alarm odorant ligand or a ligand associated with fertility. In one embodiment the compound interferes with chemosensory perception.

The invention provides a method of controlling a pest population which comprises identifying a compound by any of the methods described herein, wherein the compound interferes with an interaction between an odorant ligand and an odorant receptor which are associated with fertility.

The invention provides a composition which comprises a compound identified by any of the methods described herein and a carrier.

The invention provides a method of preparing a composition which comprises identifying a compound by any of the methods described herein and admixing a carrier. The invention provides a method of preparing a composition which comprises identifying a compound by any of the methods described herein, recovering the compound free from the receptor, and admixing a carrier. The invention provides a method of preparing a composition which comprises identifying a compound by any of the methods described herein, recovering the compound from the cells or membrane fraction or receptor protein, and admixing a carrier. Examples of carriers include, but are not limited to, phosphate buffered saline, physiological saline, water, and emulsions, such as oil/water emulsions.

The invention provides a use of a compound identified by any of the methods described herein for preparing a composition for controlling a pest population in an area by spraying the area with the compound. In one embodiment, the compound is an alarm odorant ligand or a ligand associated with fertility. In one embodiment the compound interferes with chemosensory perception.

The invention provides a use of a compound identified by any of the methods described herein for preparing a composition for controlling a pest population. In one embodiment, the compound interferes with an interaction between an odorant ligand and an odorant receptor which are associated with fertility. In one embodiment the compound interferes with chemosensory perception.

This invention will be better understood from the Experimental Procedures which follow. However, one skilled in the art will readily appreciate that the specific methods and results discussed are merely illustrative of the invention as described more fully in the claims which follow thereafter.

### Experimental Details

### Materials And Methods

### Experimental Animals

Drosophila stocks were reared on standard cornmeal-agar-molasses medium at 25° C. Oregon R strains were used for in situ hybridization experiments, and yw or W1118 strains were used for transgene injections. P-element mediated germline transformations and all subsequent fly manipulations were performed using standard techniques (Rubin et al., 1985). In some cases, transgenic constructs were injected as mixtures of two constructs, and progeny of individual transformants were analyzed by polymerase chain reaction (PCR) to determine their genotype. All analyses were performed on two to five independent transgenic lines for each construct.

### Identification of Additional GR Genes

A search for novel seven transmembrane domain receptors was performed among 5660 predicted Drosophila proteins of ‘unknown function’ (Adams et al., 2000) using a transmembrane prediction program (TopPred) (von Heijne, 1992). 310 Drosophila genes were selected for in situ hybridization analysis, 20 of which were novel members of the GR gene family previously described (Clyne et al., 2000). Additional members of the GR gene family were identified using BLAST (Altschul et al., 1990) and hidden Markov model (Eddy, 1998) searches of Drosophila genome databases with existing GR members as templates. GRs were grouped into subfamilies by BLASTP comparisons (Altschul, et al., 1998) with an e value cutoff of 10⁻⁵. Sequence relationships between the GR gene family and the DOR genes were analyzed with HMMs (Eddy, 1998), CLUSTAL alignments and neighbor joining trees (Saitou and Nei, 1987; Higgins and Sharp, 1988), and N×N BLASTP (Rubin et al., 2000) comparisons.

Five GR genes were isolated by PCR from proboscis CDNA using primers corresponding to the extent of the predicted coding region. Proboscis cDNA was obtained from one thousand microdissected probosces, using Dynal mRNA Direct (610.11) and Perkin-Elmer GeneAmp (N808-0017) kits. PCR products were cloned into pGEM-T (Promega) and sequenced in their entirety, using ABI 310 or 377 sequencing systems. An antennal cDNA library (kindly provided by Dr. Leslie Vosshall) was screened (3×10⁶ inserts) with PCR probes for Gr63F1, Gr10B1, and Gr21D1, and 6 independent cDNAS of Gr63F1 were isolated and sequenced. Sequences of Gr43C1, Gr47A1, Gr58A3, and Gr59E1 matched the previously reported sequences (Clyne et al., 2000), and sequences of Gr10B1 and Gr63F1 are included in the list above.

### In Situ Hybridization

RNA in situ hybridization was performed as previously described (Vosshall et al., 1999). Riboprobes for the 56 GR genes were generated from PCR products corresponding to predicted exons and ranged from 300-800 bp in length. Newly eclosed flies were used for in situ hybridization experiments because hybridization signals were found to be more robust at this stage.

### Construction of GR Transgenes

Generation of 15 GR promoter-Gal4 transgenes was performed as previously described (Vosshall et al., 2000). Briefly, sequences immediately adjacent to the predicted ATG initiation codon and a variable distance upstream were isolated by long range PCR with genomic DNA as template, and upstream elements were cloned into a modified CaSpeR-AUG-Gal4 vector (Vosshall et al., 2000). Regulatory element lengths for each of the GR transgenes are as follows: Gr2B1, 2.240 kB; G21D1, 9.323 kB; Gr22B1, 8.249 kB; Gr28A3, 4.245 kB; Gr32D1, 3.776 kB; Gr47A1, 7.321 kB; Gr66C1, 3.153 kB and Gr5A1, 5.156 kB; Gr10B1, 0.656 kB; Gr33C1, 3.315 kB; Gr39D2A, 8.227 kB; Gr59E2, 2.586 kB; Gr77E1, 9.502 kB; Gr93F1, 9.368 kB; Gr98A1, 1.086 kB. The first 7 transgenes drive reporter expression in chemosensory tissues; the remaining 8 transgenes were not detectably expressed in adults or larvae.

Visualization of lacZ, GFP, and nSyb-GFP Reporters

GR promoter-Gal4 lines were crossed to UAS-LacZ stocks, and whole mount heads of progeny were examined for B-galactosidase activity, following existing staining procedures (Wang et al., 1998). To enhance visualization of sensilla in the proboscis labellum, probosces were bisected and pseudotracheae were removed by microdissection. Images were recorded using a Nikon SPOT-RT digital microscope system equipped with differential interference contrast.

Progeny resulting from crosses of GR promoter-Gal4 to UAS-GFP were examined for GFP expression by direct flourescence microscopy. Adult organs and live larvae were mounted in glycerol using small coverslips as spacers and GFP flourescence was recorded with a BioRad 1024 confocal microscope.

To visualize axonal projections of GR-bearing neurons, GR promoter -Gal4 flies were mated with UAS-nSyb-GFP, and brains of F1 progeny were examined by flourescent immunohistochemistry. Larval brains were dissected and antibody staining was carried out as described in (Vosshall et al., 2000). Expression of nSyb-GFP was visualized with a rabbit anti-GFP antibody (Molecular Probes) and a goat anti-rabbit secondary antibody coupled to Alexa Fluor 488 (Molecular Probes). The nc82 monoclonal antibody (Laissue et al., 1999) was used to label brain neuropil and was visualized with goat anti-mouse IgG coupled to CY3 (Jackson ImmunoResearch). Cell nuclei were counterstained with TOTO-3 (Molecular Probes). Images were analyzed with a BioRad 1024 confocal microscope.

### Results

### A Large Family of Candidate Chemoreceptors

A novel family of putative seven transmembrane domain proteins was recently identified in searches of the Drosophila genome (Clyne et al., 2000). Analysis of a database representing 60% of the Drosophila genome identified twenty-three full-length genes and 20 partial sequences. The expression of 19 genes was examined by RT-PCR analysis and revealed 18 transcripts in the proboscis labellum, suggesting that this novel gene family may encode the fly gustatory receptors (GRs). The expression of these genes was characterized by in situ hybridization and transgene experiments and observe expression in both gustatory and olfactory chemosensory neurons in both larvae and adult flies.

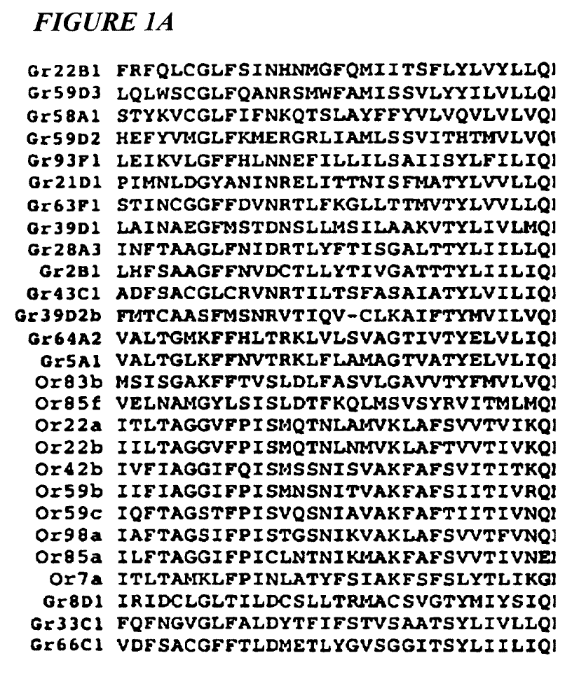

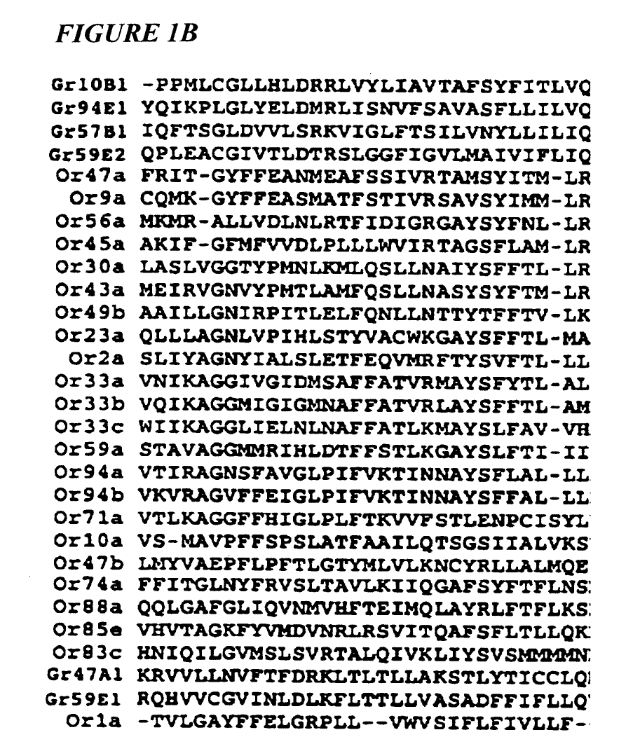

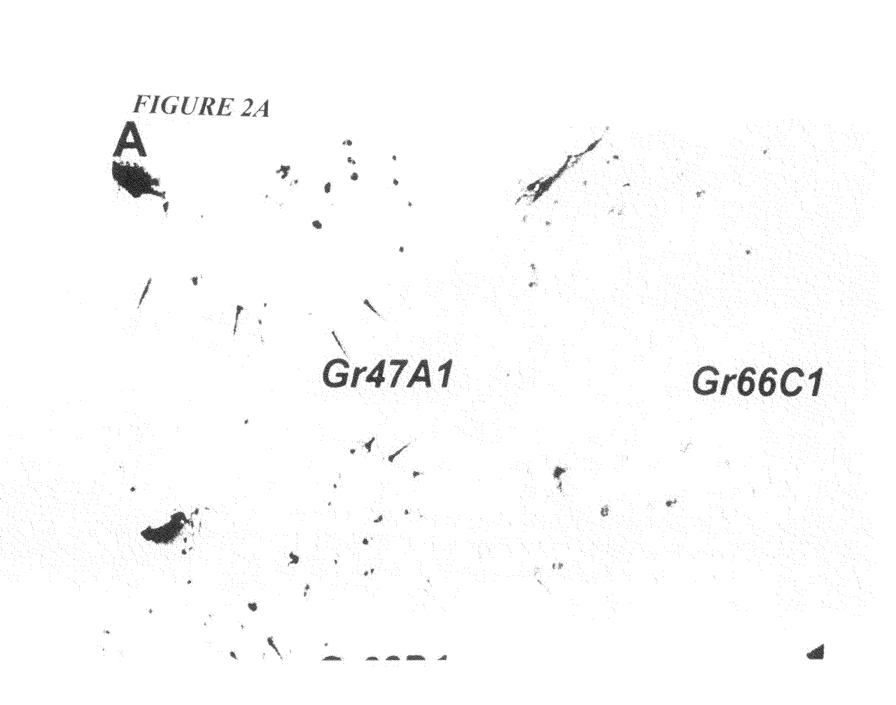

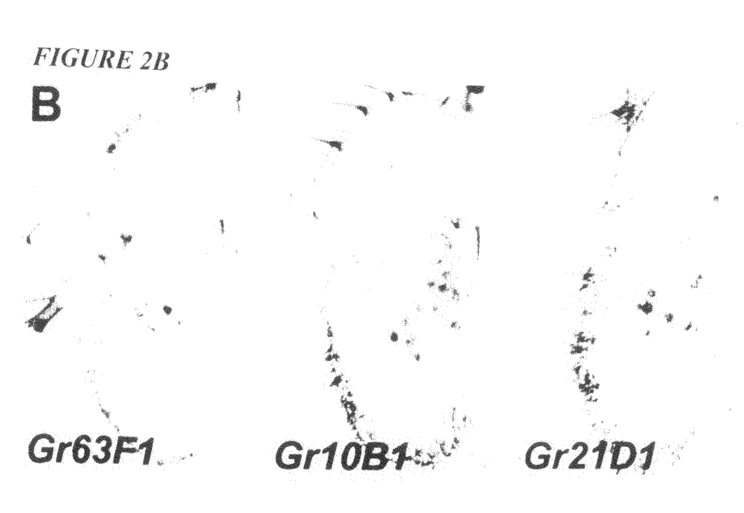

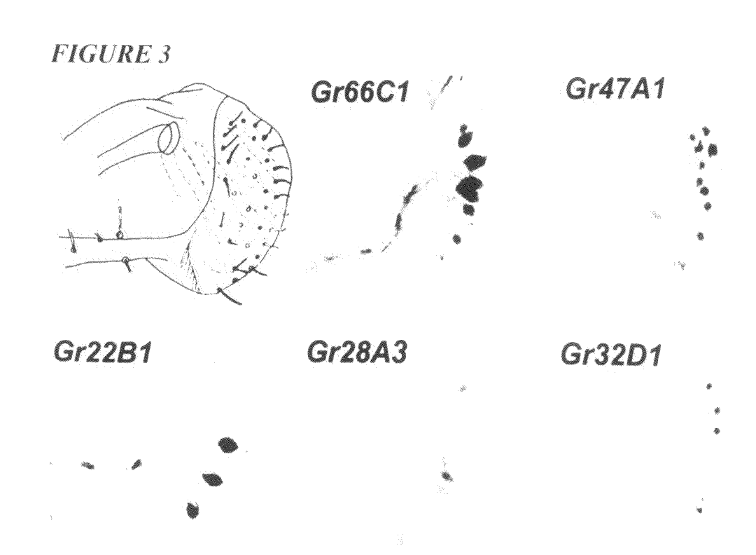

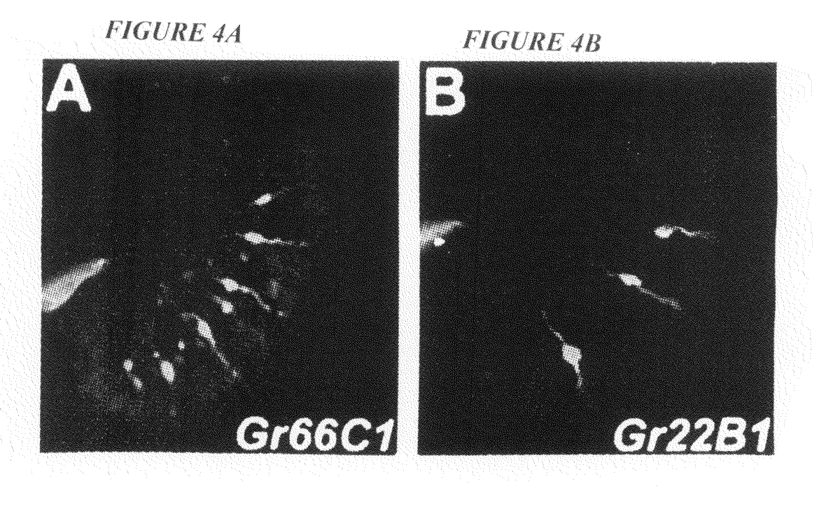

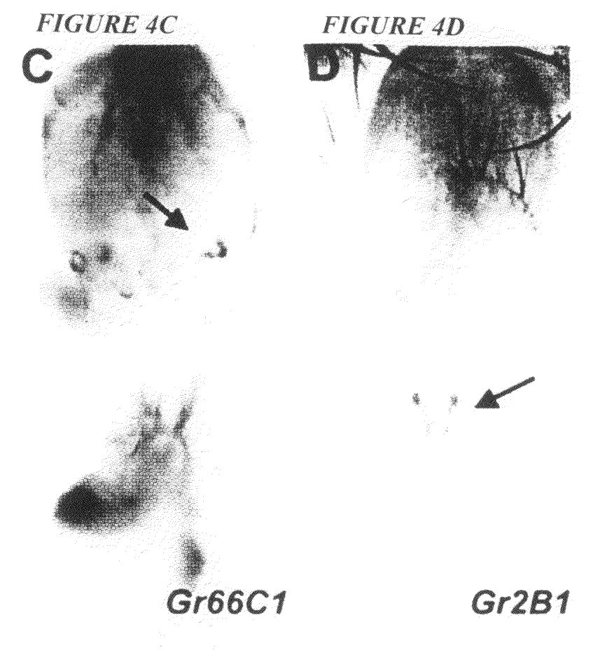

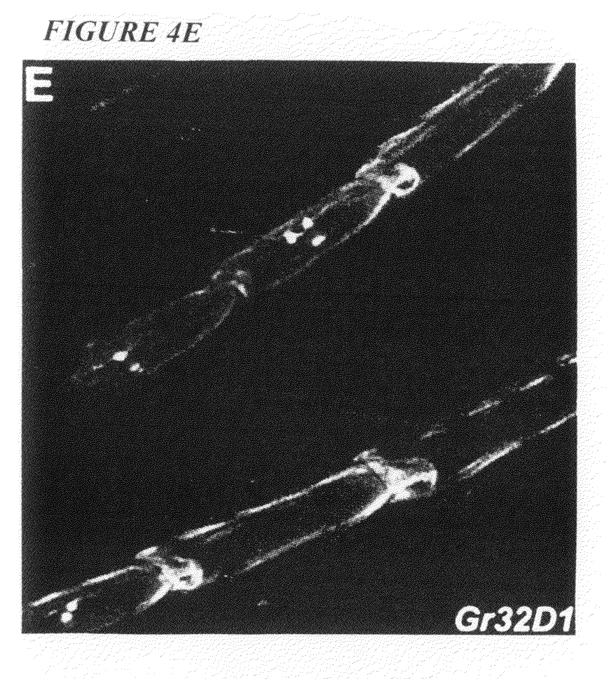

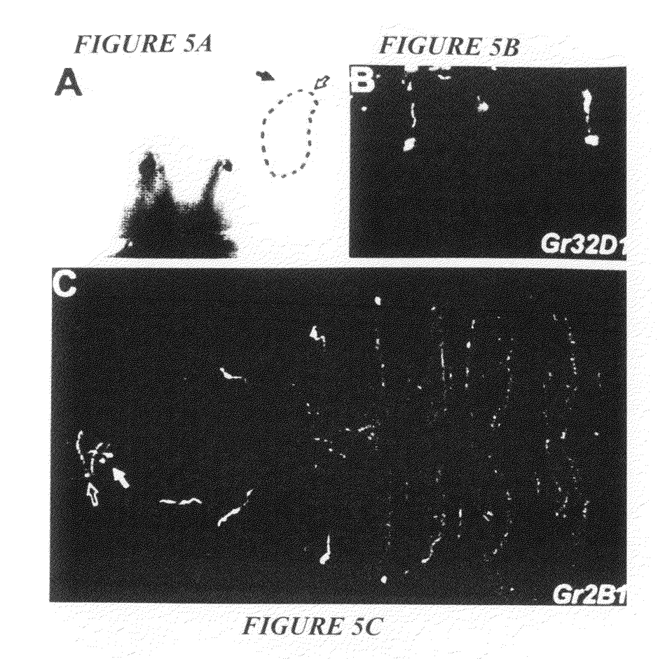

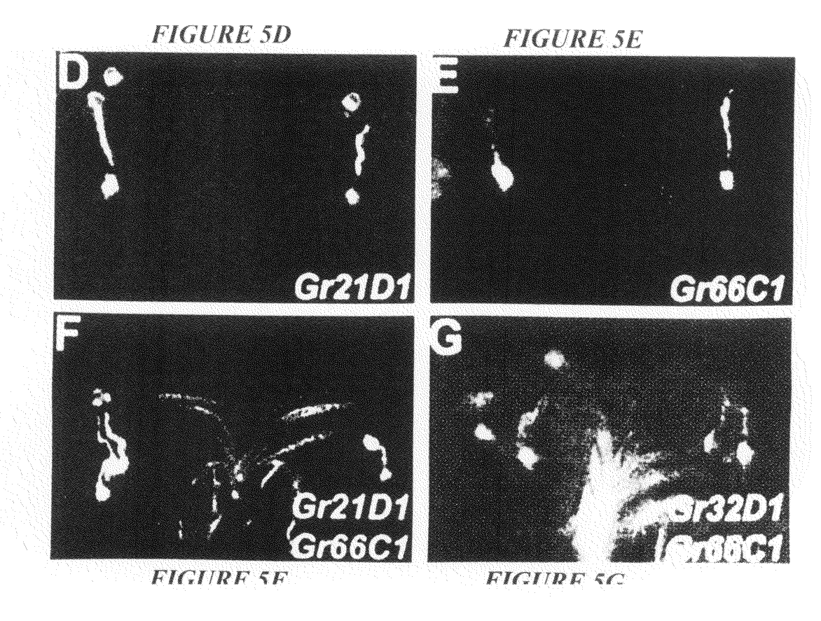

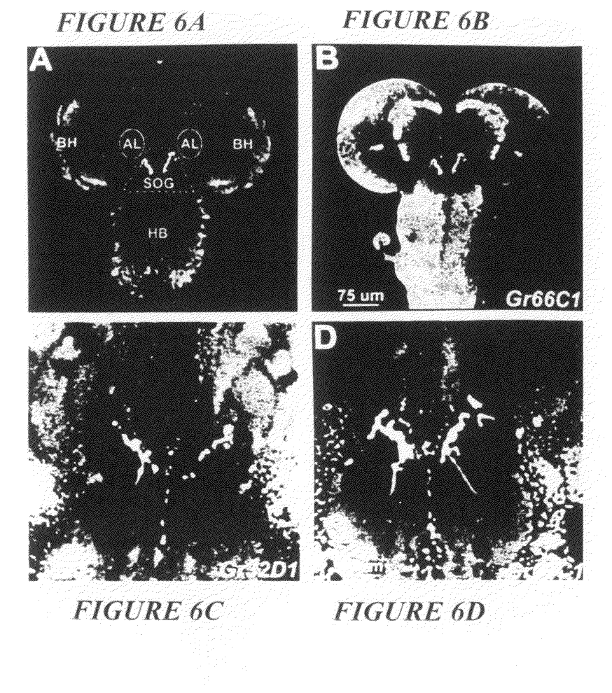

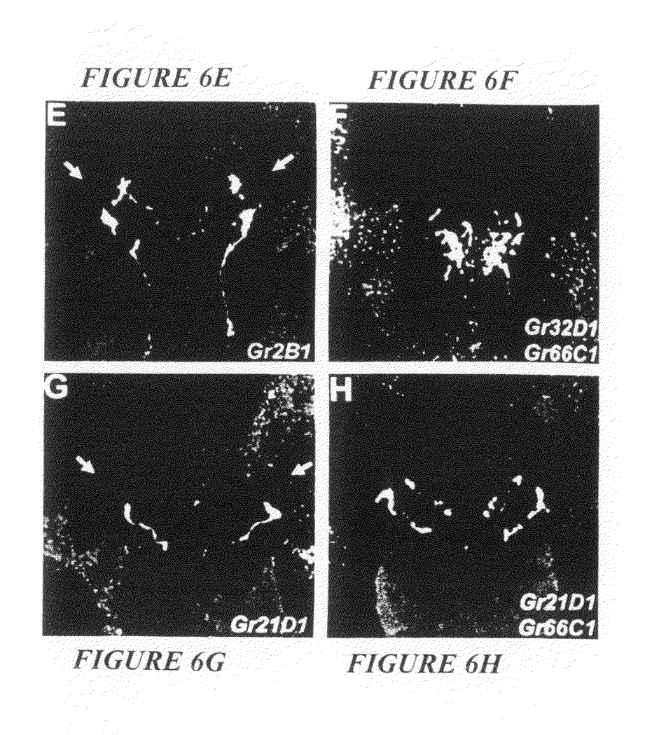

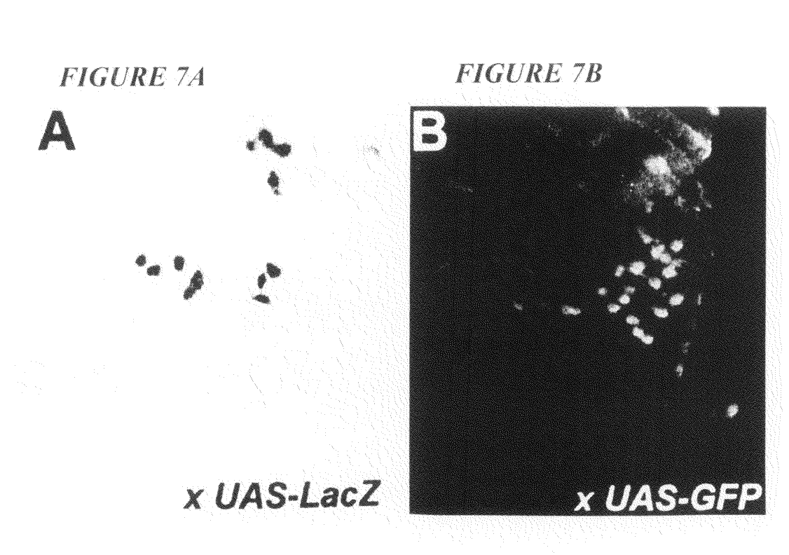

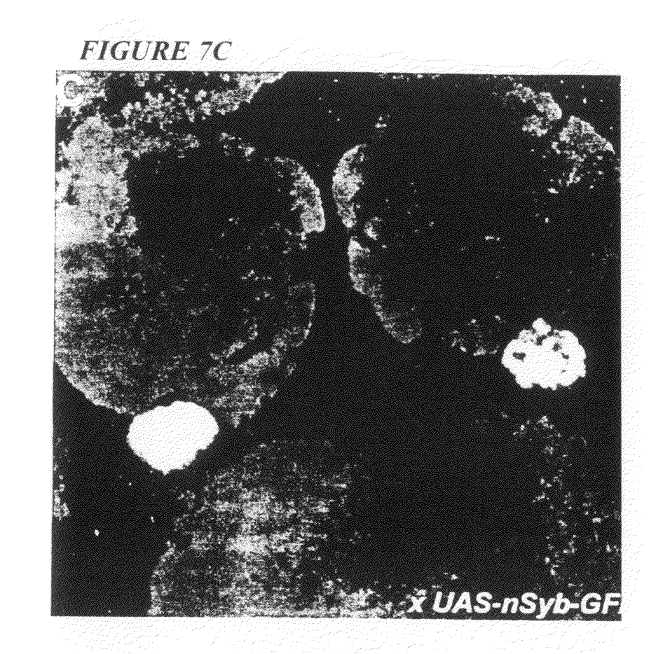

The gene family has been extended by analyzing the recently completed euchromatic genome sequence of Drosophila (Adams et al., 2000) using reiterative BLAST searches (Altschul et al., 1990), transmembrane domain prediction programs (von Heijne, 1992), and hidden Markov model (HMM) analyses (Eddy, 1998). These searches have identified a total of 56 candidate GR genes in the Drosophila genome, including 23 GRs not previously described. As originally reported, these genes encode putative seven transmembrane domain proteins of about 480 amino acids (Clyne et al., 2000). The family as a whole is extremely divergent and reveals an overall sequence identity ranging from 7-70%. However, all genes share significant sequence similarity within a 33 amino signature motif in the putative seventh transmembrane domain in the C-terminus (FIG. 1). Analysis of the sequence of the 56 genes reveals the existence of four discrete subfamilies (containing ten, six, four and three genes) whose members exhibit greater overall sequence identity ranging from 40-70%. Twenty-two of the GR genes reside as individual sequences distributed throughout each of the Drosophila chromosomes, whereas the remaining genes are linked in the genome in small tandem arrays of two to five genes.

The GR family shares little sequence similarity outside of the conserved C terminal signature in the putative seventh transmembrane domain and therefore searches of the genome database are unlikely to be exhaustive. Thus, this family of candidate gustatory receptors consists of a minimum of 56 genes. Moreover, this analysis would not detect alternatively spliced transcripts, a feature previously reported for some members of this gene family (Clyne et al., 2000). cDNAs or RT PCR products were identified from six genes; verification of the gene predictions therefore awaits the isolation and sequencing of additional cDNAs.

Interestingly, the 33 amino acid signature motif characteristic of the GR genes is present but somewhat diverged in 33 of the 70 members of the family of Drosophila odorant receptor (DOR) genes. (FIG. 1). The DOR genes, however, possess additional conserved motifs not present in the GR genes and define a distinct family (Clyne et al., 1999; Vosshall et al., 1999; Gao and Chess, 1999; Vosshall et al., 2000). These observations suggest that the putative gustatory and olfactory receptor gene families may have evolved from a common ancestral gene.

### GR Gene Expression in Olfactory and Gustatory Organs

Insight into the specific problem of the function of these candidate receptor genes and the more general question as to how tastants are recognized and discriminated by the fly brain initially requires an analysis of the patterns of expression of the individual GR genes in chemosensory cells. In situ hybridization was performed on sagittal sections of the adult fly head with RNA probes obtained from all 56 family members. Six of the genes are expressed in discrete, topographically-restricted subpopulations of neurons within the proboscis (FIG. 2A). Three of the genes revealed no hybridization to the proboscis but are expressed in spatially-defined sets of neurons within the third antennal segment, the major olfactory organ of the adult fly (FIG. 2B). The remaining genes show no hybridization to adult head tissues.

Our analysis of the pattern of GR gene expression by in situ hybridization demonstrates that a small number of GR genes is transcribed in either the proboscis or the antenna, suggesting that this family encodes chemosensory receptors involved in smell as well as taste. However, expression of over 80% of the family members was not detected using these in situ hybridization conditions. The sequence of these GR genes does not reveal nonsense or frameshift mutations that characterize pseudogenes. The inability to detect transcripts from the majority of the GR genes by in situ hybridization might result from low levels of expression of GR genes, expression in populations of chemosensory cells not amenable to analysis by in situ hybridization (e.g., leg, wing, or vulva), or expression at other developmental stages.

Lines of flies expressing GR promoter transgenes were therefore generated to visualize the expression in a wider range of cell types with higher sensitivity. Transgenes were constructed in which putative GR promoter sequences (0.5-9.5 kb of DNA immediately upstream of the translational start) were fused to the Gal4 coding sequence (Brand and Perrimon, 1993). Flies bearing GR transgenes were mated to transgenic flies that contain either B-galactosidase (lacZ) or green fluorescent protein (GFP) under the control of the Gal4-responsive promoter, UAS. CR promoter-Gal4 lines were constructed with upstream sequences from 15 chemoreceptor genes and transgene expression was detected for 7 lines (Table 1). Five of the genes that were expressed by transgene analyses were also detected by in situ hybridization.

### A Spatial Map of GR Expression in the Proboscis

Expression of the GR transgenes in the proboscis was initially visualized using the UAS-lacZ reporter. The labellum of the proboscis is formed from the fusion of two labial palps, each containing 31-36 bilaterally symmetric chemosensory bristles arranged in four rows (FIG. 3) (Arora et al., 1987; Ray et al., 1993). The sensilla of the first three columns contains four chemosensory neurons and a single mechanoreceptor cell whereas the sensilla in the most peripheral row are composed of only two chemosensory neurons and one mechanoreceptor (Nayak aria Singh, 1983; Ray et al., 1993). Each labial palp therefore contains approximately 120 chemosensory neurons.

The GR promoter-Gal4 lines were crossed to UAS-lacZ flies and the progeny were examined for lacZ expression by staining of whole mount preparations of the labial palp. Five transgenic lines exhibit lacZ expression in sensory neurons of the labial sensilla (FIG. 3). The expression of each transgene is restricted to a single row of chemosensory bristles. Gr47A1, for example, is expressed in sensilla innervating the most peripheral row of bristles, whereas Gr66C1 is expressed in sensilla that occupy the most medial column (FIG. 3). Flies bearing a GR promoter-Gal4 gene were also crossed with UAS-GFP stocks. The expression of GFP allows greater cellular definition and reveals that each receptor is expressed in a single neuron within a sensillum (FIGS. 4A, 4B). The pattern of GR gene expression determined by GR promoter transgenes resembles that seen by in situ hybridization. However, co-expression of the transgene reporter and the endogenous gene could not be directly demonstrated by dual label in situ hybridization due to low levels of GR gene expression. Nevertheless, this pattern of expression, in which a receptor is expressed in only one neuron in a sensillum and in one sensillar row, is maintained in over 50 individuals examined for each transgenic line and is also maintained in independent transformed lines for each GR transgene.

### Receptor Expression in Other Chemosensory Neurons

Chemosensory bristles reside at multiple anatomic sites in the fly including the taste organs in the mouth, the legs and wings, as well as in the female genitalia (Table 1) (Stocker, 1994). Three sensory organs reside deep in the mouth: the labral sense organ (comprised of 10 chemosensory neurons) and the ventral and dorsal cibarial organs (each containing six chemosensory neurons) (Stocker and Schorderet, 1981; Nayak and Singh, 1983). The function of these specialized sensory organs is unknown, but their anatomic position and CNS projection pattern suggests that they participate in taste recognition (Stocker and Schorderet, 1981; Nayak and Singh, 1983). Three of the five GR promoter-Gal4 lines that are expressed in the proboscis are also expressed in the cibarial organs (FIG. 4C; Table 1). One gene, Gr2B1, is expressed solely in the labral sense organ and is not detected in the proboscis labellum or in the cibarial organs (FIG. 4D).

Chemosensory bristles also decorate both the legs and wings of Drosophila with about 40 chemosensory hairs on each structure (Nayak and Singh, 1983; Hartenstein and Posakony, 1989). One gene, Gr32D1, expressed both in the proboscis and cibarial organ, is also expressed in two to three neurons in the most distal tarsal segments of all legs (FIG. 4E). These results are consistent with the observation that exposure of the legs to tastants results in proboscis extension and feeding behavior (Dethier, 1976). The observation that members of this gene family are expressed in the proboscis and in chemosensory cells of the internal mouth organs and leg suggests that this gene family encodes gustatory receptors.

### Expression of Gustatory Receptors in Drosophila Larvae

The expression of GR transgenes in larvae was also examined. The detection of food in larvae is mediated by chemosensors that reside largely in the antennal-maxillary complex, a bilaterally symmetric anterior structure composed of the dorsal and terminal organs (FIG. 5A; Table 1) (Stocker, 1994; Campos-Ortega and Hartenstein, 1997; Heimbeck et al., 1999). Each of the two larval chemosensory organs comprises about 40 neurons. Neurons of the dorsal organ primarily detect volatile odorants (Stocker, 1994), whereas the terminal organ is thought to detect both soluble and volatile chemical cues (Heimbeck et al., 1999).

The possiblity that members of the GR family are expressed in larval chemosensory cells was addressed by examining the larval progeny that result from crosses between GR promoter-Gal4 and UAS-GFP flies. Examination of live larvae by direct fluorescent microscopy reveals that five of the seven GRs expressed in the adult are expressed in single neurons within the terminal organ (FIG. 5 and Table 1). GR-promoter fusions from each of the 5 genes show bilateral expression of GFP both in the neuronal cell body and in the dendrite. The dendrites extend anteriorly to terminate in the terminal organ, a dome-shaped structure that opens to the environment. In about 5% of the larvae, a second positive cell is observed in each of the lines.

Gr2B1 is expressed in only a single neuron in the labral sense organ of the adult, but is expressed in an extensive population of chemosensory cells in larvae. This gene is expressed in two neurons innervating the dorsal organ, one neuron innervating the terminal organ, and a single bilaterally symmetric neuron innervating the ventral pit in each thoracic hemisegment (FIG. 5C). The ventral pit contains a single sensory neuron that may be involved in contact chemosensation. The GR genes are therefore likely to play a significant role in chemosensory recognition in larvae as well as adults.

The Diversity of GR expression in Individual Neurons

Olfactory neurons of mammals as well as Drosophila express a single odorant receptor such that the brain can discriminate odor by determining which neurons have been activated (Ngai et al., 1993; Ressler et al., 1993; Vassar et al., 1993; Chess et al., 1994; Gao et al., 2000; Vosshall et al., 2000). In contrast, nematode olfactory neurons and mammalian gustatory cells co-express multiple receptor genes (Bargmann and Horvitz, 1991; Troemel et al., 1995; Hoon et al., 1999; Adler et al., 2000). The diversity of GR gene expression in individual larval taste neurons was therefore examined. In larvae, most receptors are expressed in only one neuron in the terminal organ. Crosses between five GR promoter-Gal4 lines and flies bearing UAS-GFP reveal a single intensely stained neuron within each terminal organ. Seven lines bearing two different GR promoter-Gal4 transgenes along with the UAS-GFP reporter were then generated. In every line bearing two CR promoter-Gal4 fusions, two GFP positive cells per terminal organ were observed (FIGS. 5F, 5G). These experiments demonstrate that individual gustatory neurons of larvae express different complements of receptors and are likely to respond to different chemosensory cues.

### The Projections of Larval Chemosensory Neurons to the Brain

In other sensory systems, a spatial map of receptor activation in the periphery is maintained in the brain such that the quality of a sensory stimulus may be encoded in spatially defined patterns of neural activity. GR promoter-Gal4 transgenes were therefore used to drive the expression of UAS-nSyb-GFP to visualize the projections of sensory neurons expressing different GR genes. nSyb-GFP is a C-terminal fusion of green fluorescent protein to neuronal synaptobrevin that selectively labels synaptic vesicles, allowing the visualization of terminal axonal projections (Estes et al., 2000). Whole mount brain preparations from transgenic flies were examined by immunofluorescence with an antibody against GFP and a monoclonal antibody, nc82, which labels neuropil and identifies the individual glomeruli in the antennal lobe (Laissue et al., 1999). These experiments were initially performed with larvae because of the relative simplicity of the larval brain and the observation that a given GR is expressed in only a small number of gustatory neurons.

The Drosophila larval brain is composed of two dorsal brain hemispheres fused to the ventral hindbrain (FIG. 6A). The brain hemispheres and the hindbrain contain an outer shell of neuronal cell bodies and a central fibrous neuropil. Determination of the number of neuroblasts and the number of cell divisions suggest that there are approximately 10,000-15,000 neurons in the larval brain, a value 10-20 fold lower than in the adult (Hartenstein and Campos-Ortega, 1984; Hartenstein et al., 1987; Truman et al., 1993). Chemosensory neurons send axonal projections to two distinct regions of the larval brain, the antennal lobe and the subesophageal ganglion (SOG) (Stocker, 1994; Heimbeck, et al., 1999). The antennal lobe is a small neuropil in the medial aspect of the deuterocerebrum within each brain hemisphere. The antennal lobe receives input from neurons of the dorsal and terminal organ and presumably participates in processing olfactory information. The SOG resides in the most anterior aspect of the hindbrain, at the juncture of the hindbrain with the brain hemispheres. The SOG receives input from the terminal organ and mouthparts and is thought to process gustatory information. Whereas the projections of populations of chemosensory cells have been traced to the antennal lobe and the SOG, the patterns of axonal projections for individual sensory cells have not been described. Moreover, the connections of chemosensory axons with second order brain neurons is unknown for the larval brain.

Gr32D1-Gal4 is expressed in multiple neurons in the proboscis of the adult, but it is expressed in only a single neuron in the terminal organ of larvae (FIG. 5B). In larvae containing the Gr32D1-Gal4 and UAS-nSyb-GFP transgenes, it is possible to visualize the axons of Gr32D1-expressing cells as they course posteriorly to enter the subesophageal ganglion (data not shown). The axons then turn dorsally and intensely stained fibers terminate in the medial aspect of the SOG (FIG. 6C). A similar pattern is observed for neurons expressing Gr66C1 (FIG. 6B, D), a gene expressed in the proboscis in the adult and in a single neuron in the terminal organ and two in the mouth of larvae (FIG. 5E). However, the terminal arbors of Gr66C1 neurons are consistently thicker than that observed for Gr32D1, perhaps reflecting the increased number of Gr66C1-bearing neurons. The reporter nSyb-GFP stains axons only weakly but shows intense staining of what is likely to be terminal projections of sensory neurons that synapse on second order neurons in the neuropil of the SOG. This terminal arbor extends for about 40 um and reveals a looser, more distributed pattern that the tight neuropil of the olfactory glomerulus. The position and pattern of the terminal projections from individual chemosensory cells in the terminal organ show bilateral symmetry and are maintained in over 20 larvae examined.

A more complex pattern of projections is observed for Gr2B1, a gene expressed in one neuron in the terminal organ, two in the dorsal organ, and a single bilaterally symmetric neuron in each thoracic hemisegment (FIG. 5C). One set of fibers appears to terminate in the antennal lobe (FIG. 6E). A second more posterior set of fibers can be traced from the thorax into the hindbrain, with fibers terminating posterior to the antennal lobe (FIG. 6E). This pattern of projections is of interest for it implies that neurons in different locations in larvae that express the same receptor project to discrete locations in the larval brain, suggesting the possibility that the same chemosensory stimulus can elicit distinct behavioral outputs.

An attempt was made to determine whether neurons in the terminal organ that express different GRs project to discrete loci within the SOG. Larvae that express two promoter fusions, Gr66C1-Gal4 and Gr32D1-Gal4, along with a UAS-nSyb-GFP transgene were generated. The projections in these flies are broadened, suggesting that these sets of neurons terminate in overlapping but non-identical regions of the SOG (FIG. 6F). More definitive data to support the existence of a topographic map of taste quality will require two color labelling of the different fibers to discern whether the projections from neurons expressing different GRs are spatially segregated in the SOG.

Are GRs also Odorant Receptors?

A large family of presumed olfactory receptor genes in Drosophila (the DOR genes) has been identified that as distinct from the GR gene family (Clyne et al., 1999; Gao and Chess, 1999; Vosshall et al., 1999; Vosshall et al., 2000). Expression of the DOR genes is only observed in olfactory sensory neurons within the antenna and maxillary palp, where a given DOR gene is expressed in a spatially invariant subpopulation of cells (Clyne et al., 1999; Gao and Chess, 1999; Vosshall et al., 1999; Vosshall et al., 2000). In situ hybridization experiments demonstrate that three members of the GR gene family are also expressed in subpopulations of antennal neurons (FIG. 2B). These observations suggest either that the odorant receptors in Drosophila are encoded by at least two different gene families or that previously unidentified taste responsive neurons reside within the antenna.

In Drosophila, olfactory information is transmitted to the antennal lobe, whereas gustatory neurons in the proboscis and mouth relay sensory information to the subesophageal ganglion (Stocker, 1994). The spatial pattern of expression of GRs in the antenna and the pattern of projections of their sensory axons in the brain were therefore examined. In situ hybridization with the three GR genes reveals that each gene is expressed in about 20-30 cells/gene in the antenna (FIG. 2B). Similar results are obtained in a cross between an antennal GR promoter-Gal4 line, Gr21D1-Gal4, and UAS-LacZ or UAS-GFP lines (FIGS. 7A, 7B). This pattern of GR gene expression is maintained in over 50 antennae that have been analyzed. The GR-positive cells occupy regions of the antenna that do not express identified members of the DOR gene family (Vosshall et al., 2000), suggesting that there is spatial seggregation of these two receptor families.

Whether antennal neurons expressing a GR gene project to the antennal lobe in a manner analogous to that observed for cells expressing the DOR genes was next addressed. Transgenic flies expressing a Gr21D1 promoter-Gal4 fusion were crossed to animals bearing the UAS-nSyb-GFP transgene. These studies demonstrate that neurons expressing the Gr21D1 transgene project to a single, bilaterally symmetric glomerulus in the ventral-most region of the antennal lobe (the V glomerulus) (FIG. 7C) (Stocker et al., 1990; Laissue et al., 1999) and do not project to the SOG. Thus, as in the case of the family of DOR genes (Gao et al., 2000; Vosshall et al., 2000), neurons expressing the same receptor project to a single spatially invariant glomerulus.

Gr21D1 is also expressed in one cell of the terminal organ of larvae (FIG. 5D). The projections of Gr21D1-bearing neurons were therefore traced to the larval brain. Gr21D1 axons enter the larval brain and terminate in the antennal lobe rather than the SOG (FIG. 6G). The segregation of projections from presumed olfactory and gustatory neurons is apparent in larvae that contain Gr21D1-Gal4 and Gr66C1-Gal4 along with UAS-nSyb-GFP. In these transgenic flies, two distinct sets of termini are observed, one entering the SOG, and a second entering the antennal lobe (FIG. 6H).

Thus, a member of the GR genie family is expressed in sensory neurons of the antenna and the terminal organ of larvae, and GR-bearing neurons project to the antennal lobe. These data indicate that at least two independent gene families, the DORs and the GRs, recognize olfactory information. The GR gene family is therefore likely to encode both olfactory and gustatory receptors, and neurons expressing distinct classes of GR receptors project to different regions of the fly brain.

Table 1. Summary of Drosophila chemosensory tissues and GR transgene expression patterns.

The table summarizes the expression patterns of GR promoter-Gal4 transqenes in adult and larval chemosensory tissues. Adult Drosophila sense gustatory cues with chemosensory bristles on the labellum of the proboscis, legs and wings, and with specialized structures of the internal mouthparts, the cibarial organs and the labral sense organ. Gustatory neurons on the proboscis send axonal projections to the subesophageal ganglion (SOG). Sensory neurons on the antenna recognize olfactory cues and project to the antennal lobe (AL). In Drosophila larvae, gustatory cues are recognized by neurons innervating the terminal organ and possibly the ventral pits, and olfactory cues are recognized by neurons innervating the dorsal organ and the terminal organ. Gustatory tissues are highlighted in blue and olfactory tissues are highlighted in pink. The schematic of the adult fly is adapted from Stocker (1994). The schematic of the larva is adapted from Struhl (1981).

TABLE 1
Expression profiles of GR transgenes

| | ADULT | | | | | | LARVA | | | | |
|---|---|---|---|---|---|---|---|---|---|---|---|
| | In situ | | | cibarial | labral | | terminal | dorsal | | | ventral |
| GR | signal | labellum | antenna | organs | organ | leg | organ | organ | mouth | gut | pits |
| Gr2B1 | − | − | − | − | + | − | + | + | − | + | + |
| Gr21D1 | antenna | − | + | − | − | − | + | − | − | − | − |
| Gr22B1 | − | + | − | − | − | − | − | − | − | − | − |
| Gr28A1 | labellum | + | − | + | − | − | + | − | + | − | − |
| Gr32D1 | labellum | + | − | + | − | + | + | − | − | − | − |
| Gr47A1 | labellum | + | − | − | − | − | − | − | − | − | − |
| Gr66C1 | labellum | + | − | + | − | − | + | − | + | − | − |

### DISCUSSION

### A Family of Gustatory and Olfactory Receptors

Specialized sense organs have evolved to recognize chemosensory information in the environment. The antennae in insects, the amphid in nematodes, and the nose of mammals allow the recognition of a vast repertoire of volatile odorants often over long distances. Taste organs have evolved to accommodate a distinct function, the recognition of soluble chemical cues over shorter distances. In vertebrates, taste is largely restricted to the tongue and palate, whereas in insects, gustatory neurons are more broadly distributed along the body plan and reside not only in the proboscis and pharynx but also on the wings, legs, and female genitalia. Anatomic and functional segregation of the gustatory and olfactory systems is not only apparent in the peripheral receptor field but in the projections to the brain. In the fly, for example, olfactory neurons project to the antennal lobe, whereas most gustatory neurons ultimately synapse within the subesophageal ganglion. This separation is also observed in vertebrates where taste and smell are accommodated by distinct sense organs and conveyed to different brain regions by different cranial nerves. Thus, a common sensory function, the recognition of chemical cues, has undergone specialization to allow for the recognition of at least two distinct categories of chemosensory information, each eliciting distinct behavioral responses.

This study has characterized the patterns of expression of a large family of genes in Drosophila that are likely to encode both odorant and gustatory receptors. A family of candidate taste receptors was identified by searching the Drosophila genome with an algorithm designed to detect genes encoding seven transmembrane domain proteins (Clyne et al., 2000). This analysis was extended through a search of the complete euchromatic genome of Drosophila and identify 56 genes within the family. All of the CR genes contain a signature motif in the carboxyl terminus that is also present within some members of the DOR gene family, suggesting that these two families share a common origin.

The GR family of proteins was tentatively identified as gustatory receptors solely on the basis of PCR analysis of proboscis RNA (Clyne et al., 2000). In situ hybridization and transgene experiments demonstrate that members of this gene family are expressed in the antennae, proboscis, pharynx, leg, and larval chemosensory organs. Thus, a single gene family encodes chemosensory receptors containing both olfactory and gustatory receptors. Flies bearing GR promoter transgenes were generated from 15 GR genes. Expression is observed in seven lines and is restricted to chemosensory cells. No expression is detected in other neurons or in non-neuronal cells. These data suggest that the expression of this family is limited to gustatory and olfactory neurons, and that the inability to observe expression in 8 transgenic lines perhaps reflects the structural inadequacy of the promoters.

A common gene family encoding both olfactory and taste receptors is not present in vertebrates where the main olfactory epithelium, the vomeronasal organ and the tongue express receptors encoded by independent gene families (Buck and Axel, 1991; Dulac and Axel, 1995; Herrada and Dulac, 1997; Matsunami and Buck, 1997; Ryba and Tirindelli, 1997; Hoon et al., 1999; Adler et al., 2000; Matsunami et al., 2000). The observations described herein are more reminiscent of the chemosensory receptor families in C. elegans that encode odorant receptors expressed in the amphid neurons and taste receptors in sensory neurons responsive to soluble chemicals (Troemel et al., 1995; Troemel, 1999).

### Patterns of GR Gene Expression and Taste Modalities

The size of the family of candidate taste receptors and the pattern of expression in chemosensory cells provides insight into the problem of the recognition and discrimination of gustatory cues. On average, each GR is expressed in 5% of the cells in the proboscis labellum, suggesting that the proboscis alone will contain at least 20 distinct taste cells expressing about 20 different GR receptors. Moreover, a given receptor is expressed in one of the four rows of sensilla such that the sensilla in different rows are likely to be functionally distinct. Electrophysiologic studies have suggested that all sensilla are identical and contain four distinct cells each responsive to a different category of taste (Dethier, 1976; Rodriques and Siddiqi, 1978; Fujishiro et al., 1984). The data presented herein are not consistent with these conclusions and argue that different rows of sensilla are likely to contain cells with different taste specificities.

At present the nature of the ligands recognized by these GR receptors are not known, nor is it known whether all taste modalities are recognized by this gene family. In mammals, gustatory cues have classically been grouped into five categories: sweet, bitter, salt, sour and glutamate (umami) (Kinnamon and Margolskee, 1996; Lindemann, 1996; Gilbertson et al., 2000). Sugar and bitter taste are likely to be mediated by G protein-coupled receptors since these modalities require the function of a taste cell specific Gₐ subunit, gustducin (McLaughlin et al., 1992; Wong et al., 1996). Recently, two novel families of seven transmembrane proteins (the T1Rs and T2Rs) were shown to be selectively expressed in taste cells in the tongue and palate epithelium (Hoon et al., 1999; Adler et al., 2000; Matsunami et al., 2000). Genetic experiments implicated members of the T2R family in the recognition of bitter tastants (Adler et al., 2000; Matsunami et al., 2000) and functional studies directly demonstrated that members of the T2R family serve as gustducin-linked bitter taste receptors. (Chandrashekar et al., 2000). A large number of candidate genes have been suggested to encode receptors for other taste modalities but in only a few instances have functional data and expression patterns supported these assumptions. In mammals, an amiloride-sensitive sodium channel has been suggested as the salt receptor (Heck et al., 1984), a degenerin homolog (MDEG-1) (Ugawa et al., 1-998) and a potassium channel (Kinnamon et al., 1988) as sour or pH sensors, and a rare splice form of the metabotropic glutamate receptor as the umami sensor (Chaudhari et al., 2000). In Drosophila, genetic analysis of mutant flies defective in the recognition of the sugar, trehalose, has led to the identification of a transmembrane receptor distinct from GRs that reduces the sensitivity to one class of sugars (Ishimoto et al., 2000). The interpretation of the role of these putative taste receptors in taste perception awaits a more definitive association between tastant and gene product.

### The Logic of Taste Discrimination

How does the fly discriminate among multiple tastants? One mechanism of chemosensory discrimination, thought to operate in the olfactory system of insects and vertebrates, requires that individual sensory neurons express only one of multiple receptor genes (Buck and Axel, 1991; Ngai et al., 1993; Ressler et al., 1993; Vassar et al., 1993; Chess et al., 1994; Clyne et al., 1999; Gao and Chess, 1999; Vosshall et al., 1999). Neurons expressing a given receptor project axons that converge on topographically invariant glomeruli such that different odors elicit different patterns of spatial activity in the brain (Ressler et al., 1994; Vassar et al., 1994; Mombaerts et al., 1996; Wang et al., 1998; Gao et al., 2000; Vosshall et al., 2000). The nematode C. elegans uses a rather different logic, in which a given sensory neuron dictates a specific behavior but expresses multiple receptors (Bargmann and Horvitz, 1991; Troemel et al., 1995; Troemel et al., 1997). In the worm olfactory system, discrimination is necessarily more limited and exploits mechanisms to diversify the limited number of sensory cells (Colbert and Bargmann, 1995; Troemel et al., 1999; L'Etoile and Bargmann, 2000). A similar logic has been suggested for mammalian taste. Several members of the T2R family of about 50 receptor genes, each thought to encode bitter sensors, are co-expressed in sensory cells within the tongue (Adler et al., 2000). This organization allows the organism to recognize a diverse repertoire of aversive tastants but limits the ability to discriminate among them.

What can be discerned about the logic of taste discrimination from the pattern of GR gene expression in Drosophila? First, the number of GR genes, 56, approximates the number of DOR genes, suggesting that the fly recognizes diverse repertoires of both soluble and volatile chemical cues. Moreover, the data presented herein argue that individual sensory neurons differ with respect to receptor gene expression and are therefore functionally distinct. Experiments with Drosophila larvae demonstrate that a given GR gene is expressed in one neuron in the larval terminal organ. Strains bearing two different GR-promoter fusions reveal twice the number of expressing cells. Similar results are obtained in adult gustatory organs (data not shown). More definitive experiments to examine the diversity of receptor expression in a single neuron, employed successfully in the olfactory system, have been difficult since the levels of GR RNA are 10-20 fold lower than odorant receptor RNA levels. Nevertheless, experiments described herein demonstrate that different gustatory neurons express different complements of GR genes and at the extreme are consistent with a model in which gustatory neurons express only a single receptor gene.

How does the brain discern which of the different gustatory neurons is activated by a given tastant? As in other sensory systems, it is possible that axons from different taste neurons segregate to spatially distinct loci in the subesophageal ganglion. In such a model, taste quality would be represented by different spatial patterns of activity in the brain. Preliminary experiments suggest that neurons expressing different GRs project to spatially segregated loci within the brain. Clear segregation of axonal termini is observed for presumed taste neurons that project to the SOG and olfactory neurons that project to the antennal lobe. A second interesting pattern of projections is observed for the presumed gustatory receptor Gr2B1, a gene expressed in neurons in the terminal and dorsal organs and in a single neuron in the ventral pit present bilaterally in each thoracic segment. At least two spatially segregated targets are observed for these neurons in the larval brain: one set of fibers terminates in glomeruli of the antennal lobe and a second set of fibers (from the ventral pits) project to the SOG. Thus, neurons expressing the same receptor in different chemosensory organs project to distinct brain regions. In this manner, the same chemosensory cue could elicit distinct behaviors depending upon the cell it activates. Sucrose, for example, could ellicit chemoattraction upon exposure to the thoracic neurons and eating behavior upon activation of neurons in the terminal and dorsal organ.

These data establish that presumed olfactory neurons and gustatory neurons expressing GR genes project to different regions of the larval brain. Taste neurons expressing different GR genes, however, all project to the SOG. The current data do not permit us to discern whether axons from neurons expressing different GR genes project to spatially distinct loci within the SOG. The axon termini of gustatory neurons terminate in more diffuse, elongated structures than the tightly compacted glomeruli formed by olfactory sensory axons, rendering it difficult at present to discern a topographic map of gustatory projections in the larval brain.

### Sensory Perception in Larvae

Insects provide an attractive model system for the study of chemosensory perception because they exhibit sophisticated taste and olfactory driven behaviors that are controlled by a chemosensory system that is anatomically and genetically simpler than vertebrates (Nassif et al., 1998). Drosophila larvae afford a particularly facile organism because much of their behavior surrounds eating. Gustatory neurons in the terminal organ and along the body plan, together with olfactory sensory cells in the dorsal and terminal organs, combine to identify food sources and elicit eating behaviors (Stocker, 1994).

Members of the Drosophila odorant receptor (DOR) family are expressed in the adult olfactory system but cannot be detected in larval chemosensory organs. GR genes are expressed in larval olfactory and gustatory neurons and may encode the entire repertoire of larval chemosensory receptors. The simplicity of the Drosophila larvae, coupled with the ease of behavioral studies, suggests that it may be possible to relate the recognition of chemosensory information to specific behavioral responses and ultimately to associate changes in behavior with modifications in specific connections.

### REFERENCES

Adams, M. et al. (2000). The genome sequence of Drosophila melanogaster. Science 287, 2185-2195.
Adler, E., Hoon, M. A., Mueller, K. L., Chandrashekar, J., Ryba, N. J. and Zuker, C. S. (2000). A novel family of mammalian taste receptors. Cell 100, 693-702.
Altschul, S., Madden, T., Schaffer, A., Zhang, J., Zhang, Z., Miller, W., and Lipman, D. (1997). Gapped BLAST and PSI-BLAST: A new generation of protein database search programs. Nucleic Acids Res 25, 3389-402.
Altschul, S. F., Gish, W., Miller, W., Myers, E. W. and Lipman, D. J. (1990). Basic local alignment search tool. J. Mol. Biol. 215, 403-410.
Arora, K., Rodrigues, V., Joshi, S., Shanbhag, S. and Siddiqi, O. (1987). A gene affecting the specificity of the chemosensory neurons of Drosophila. Nature 330, 62-63.
Bargmann, C. I. and Horvitz, H. R. (1991). Chemosensory neurons with overlapping functions direct chemotaxis to multiple chemicals in C. elegans. Neuron 7, 729-742.
Brand, A. H. and Perrimon, N. (1993). Targeted gene expression as a means of altering cell fates and generating dominant phenotypes. Development 118, 401-415.
Buck, L. and Axel, R. (1991). A novel multigene family may encode odorant receptors: a molecular basis for odor recognition. Cell 65, 175-187.
Campos-Ortega, J. A. and Hartenstein, V. (1997). The embryonic development of Drosophila Melanogaster. Berlin, Springer.
Chandrashekar, J., Mueller, K. L., Hoon, M. A., Adler, E., Feng, L., Guo, W., Zuker, C. S. and Ryba, N. J. (2000). T2Rs function as bitter taste receptors. Cell 100, 703-711.
Chaudhari, N., Landin, A. M. and Roper, S. D. (2000). A metabotropic glutamate receptor variant functions as a taste receptor. Nat. Neurosci. 3, 113-119.
Chess, A., Simon, I., Cedar, H. and Axel, R. (1994). Allelic inactivation regulates olfactory receptor gene expression. Cell 78, 823-834.
Clyne, P. J., Warr, C. G. and Carlson, J. R. (2000). Candidate Taste Receptors in Drosophila. Science 287, 1830-1834.
Clyne, P. J., Warr, C. G., Freeman, M. R., Lessing, D., Kim, J. and Carlson, J. R. (1999). A novel family of divergent seven-transmembrane proteins: candidate odorant receptors in Drosophila. Neuron 22, 327-338.
Colbert, H. A. and Bargmann, C. I. (1995). Odorant-specific adaptation pathways generate olfactory plasticity in C. elegans. Neuron 14, 803-812.
Dethier, V. G. (1976). The Hungry Fly Cambridge, Mass., Harvard University Press.
Dulac, C. and Axel, R. (1995). A novel family of genes encoding putative pheromone receptors in mammals. Cell 83, 195-206.
Eddy, S. R. (1998). Profile hidden Markov models. Bioinformatics 14, 755-763.

Estes, P. E., Ho, G., Narayanan, R. and Ramaswami, M. (2000). Synaptic localization and restricted diffusion of a Drosophila neuronal synaptobrevin-green fluorescent protein chimera in vivo. J. Neurogenetics 13, 233-255.
Falk, R., Bleiser-Avivi, N. and Atidia, J. (1976). Labellar Taste Organs of Drosophila Melanogaster. Journal of Morphology 150, 327-341.
Fujishiro, N., Kijima, H. and Morita, H. (1984). Impulse frequency and action potential amplitude in labellar chemosensory neurons of Drosophila Melanogaster. Journal of Insect Physiology 30, 317-325.
Gao, Q. and Chess, A. (1999). Identification of candidate Drosophila olfactory receptors from genomic DNA sequence. Genomics 60, 31-39.
Gao, Q., Yuan, B. and Chess, A. (2000). Convergent Projections of Drosophila Olfactory Neurons to Specific Glomeruli in the Antennal Lobe. Nature Neurosci. 3, 780-785.
Gilbertson, T. A., Damak, S. and Margolskee, R. F. (2000). The molecular physiology of taste transduction. Curr Opin Neurobiol 10, 519-527.
Hartenstein, V. and Campos-Ortega, J. A. (1984). Early neurogenesis in wild-type Drosophila melanogaster. Wilhelm Roux's Arch Dev Bio 193, 308-325.
Hartenstein, V. and Posakony, J. W. (1989). Development of adult sensilla on the wing and notum of Drosophila melanogaster. Development 107, 389-405.
Hartenstein, V., Rudloff E. and Campos-Ortega, J. A. (1987). The pattern of proliferation of the neuroblasts in the wild-type embryo of Drosophila melanogaster. Wilhelm Roux's Arch Dev Bio 198, 264-274.
Heck, G. L., Mierson, S. and DeSimone, J. A. (1984). Salt taste transduction occurs through an amiloride-sensitive sodium transport pathway. Science 223, 403-405.
Heimbeck, G., Bugnon, V., Gendre, N., Haberlin, C. and Stocker, R. F. (1999). Smell and taste perception in Drosophila melanogaster larva: toxin expression studies in chemosensory neurons. J. Neurosci. 19, 6599-6609.
Herrada, G. and Dulac, C. (1997). A novel family of putative pheromone receptors in mammals with a topographically organized and sexually dimorphic distribution. Cell 90, 763-773.
Higgins, D. G. and Sharp, P. M. (1988). CLUSTAL: a package for performing multiple sequence alignment on a microcomputer. Gene 73, 237-244.
Hoon, M. A., Adler, E., Lindemeier, J., Battey, J. F., Ryba, N. J. and Zuker, C. S. (1999). Putative mammalian taste receptors: a class Of taste-specific GPCRs with distinct topographic selectivity. Cell 96, 541-551.
Ishimoto, H., Matsumoto, A. and Tanimura, T. (2000). Molecular identification of a taste receptor gene for trehalose in Drosophila. Science 289, 116-119.
Kinnamon, S. C., Dionne, V. E. and Beam, K. G. (1988). Apical localization of K+channels in taste cells provides the basis for sour taste transduction. Proc. Natl. Acad. Sci. USA 85, 7023-7027.
Kinnamon, S. C. and Margolskee, R. F. (1996). Mechanisms of taste transduction. Curr. Opin. Neurobiol. 6, 506-513.
Kunishima, N., Shimada, Y., Tsuji, Y., Sato, T., Yamamoto, M., Kumasaka, T., Nakanishi, S., Jingami, H. and Morikawa, K. (2000). Structural basis of glutamate recognition by a dimeric metabotropic glutamate receptor. Nature 407, 971-977.
Laissue, P. P., Reiter, C., Hiesinger, P. R., Halter, S., Fischbach, K. F. and Stocker, R. F. (1999). Three-dimensional reconstruction of the antennal lobe in Drosophila melanogaster. J. Comp. Neurol. 405, 543-552.
L'Etoile, N. D. and Bargmann, C. I. (2000). Olfaction and Odor Discrimination Are Mediated by the C. elegans Guanylyl Cyclase ODR-1. Neuron 25, 575-586.
Lindemann, B. (1996). Taste reception. Physiol. Rev. 76, 718-766.
Matsunami, H. and Buck, L. B. (1997). A multigene family encoding a diverse array of putative pheromone receptors in mammals. Cell 90, 775-784.
Matsunami, H., Montmayeur, J. P. and Buck, L. B. (2000). A family of candidate taste receptors in human and mouse. Nature 404, 601-604.
McLaughlin, S. K., McKinnon, P. J. and Margolskee, R. F. (1992). Gustducin is a taste-cell-specific G protein closely related to the transducins. Nature 357, 563-569.
Mombaerts, P., Wang, F., Dulac, C., Chao, S. K., Nemes, A., Mendelsohn, M., Edmondson, J. and Axel, R. (1996). Visualizing an olfactory sensory map. Cell 87, 675-686.
Nassif, C., Noveen, A. and Hartenstein, V. (1998). Embryonic Development of the Drosophila Brain. I. Pattern of Pioneer Tracts. J. Comp. Neurol. 402, 10-31.
Nayak, S. V. and Singh, R. N. (1983). Sensilla on the tarsal segments and mouthparts of adult Drosophila Melanogaster. International Journal of Insect Morphology and Embryology 12, 273-291.
Ngai, J., Chess, A., Dowling, M. M., Necles, N., Macagno, E. R. and Axel, R. (1993). Coding of olfactory information: topography of odorant receptor expression in the catfish olfactory epithelium. Cell 72, 667-680.
Possidente, D. R. and Murphey, R. K. (1989). Genetic control of sexually dimorphic axon morphology in Drosophila sensory neurons. Dev. Biol. 132, 448-457.
Power, M. E. (1948). The thoracico-abdominal nervous system of an adult insect, Drosophila Melanogaster. Journal of Comparative Neurology 88, 347-409.
Rajashekhar, K. P. and Singh, R. N. (1994). Neuroarchitecture of the tritocerebrum of Drosophila melanogaster. J. Comp. Neurol. 349, 633-645.
Ray, K., Hartenstein, V. and Rodrigues, V. (1993). Development of the taste bristles on the labellum of Drosophila melanogaster. Dev. Biol. 155, 26-37.
Ressler, K. J., Sullivan, S. L. and Buck, L. B. (1993). A zonal organization of odorant receptor gene expression in the olfactory epithelium. Cell 73, 597-609.
Ressler, K. J., Sullivan, S. L. and Buck, L. B. (1994). Information coding in the olfactory system: evidence for a stereotyped and highly organized epitope map in the olfactory bulb. Cell 79, 1245-1255.
Rice, M. J. (1977). Blowfly ovipositor receptor neurons sensitive to monovalent cation concentration. Nature 268, 747-749.
Rodriques, V. and Siddiqi, O. (1978). Genetic analysis of a chemosensory pathway. Proceedings of the Indian Academy of Science, Series B 87, 147-160.
Rubin, G. M., Hazelrigg, T., Karess, R. E., Laski, F. A., Layerty, T., Levis, R., Rio, D. C., Spencer, F. A. and Zuker, C. S. (1985). Germ line specificity of P-element transposition and some novel patterns of expression of transduced copies of the white gene. Cold Spring Hare. Symp. Quant Biol. 50, 329-335.
Rubin, G. M., et al. (2000). Comparative genomics of the eukaryotes. Science 287, 2204-2215.
Ryba, N. J. and Tirindelli, R. (1997). A new multigene family of putative pheromone receptors. Neuron 19, 371-379.
Saitou, N. and Nei, M. (1987). The neighbor-joining method: A new method for reconstructing phylogenetic trees. Mol. Bio. Evol. 4, 406-425.
Shanbhag, S. R. and Singh, R. N. (1992). Functional implications of the projections of neurons from individual labellar sensillum of Drosophila Melanogaster as revealed by neuronal marker horseradish peroxidase. Cell and Tissue Research 267, 273-282.
Singh, R. N. (1997). Neurobiology of the gustatory systems of Drosophila and some terrestrial insects. Microsc. Res. Tech. 39, 547-563.
Stocker, R. F. (1994). The organization of the chemosensory system in Drosophila melanogaster: a review. Cell Tissue Res. 275, 3-26.
Stocker, R. F., Lienhard, M. C., Borst, A. and Fischbach, K. F. (1990). Neuronal architecture of the antennal lobe in Drosophila melanogaster. Cell Tissue Res. 262, 9-34.
Stocker, R. F. and Schorderet, M. (1981). Cobalt filling of sensory projections from internal and external mouthparts in Drosophila. Cell Tissue Res. 216, 513-523.
Struhl, G. (1981). A gene product required for correct initiation of segmental determination in Drosophila. Nature 293, 36-41.
Taylor, B. J. (1989). Sexually dimorphic neurons of the terminalia of Drosophila melanogaster: II. Sex-specific axonal arborizations in the central nervous system. J. Neurogenet. 5, 193-213.
Tompkins, L., Siegel, R. W., Gailey, D. A. and Hall, J. C. (1983). Conditioned courtship in Drosophila and its mediation by association of chemical cues. Behav. Genet. 13, 565-578.
Troemel, E. R. (1999). Chemosensory signaling in C. elegans. Bioessays 21, 1011-1020.
Troemel, E. R., Chou, J. H., Dwyer, N. D., Colbert, H. A. and Bargmann, C. I. (1995). Divergent seven transmembrane receptors are candidate chemosensory receptors in C. elegans. Cell 83, 207-218.

Troemel, E. R., Kimmel, B. E. and Bargmann, C. I. (1997). Reprogramming chemotaxis responses: sensory neurons define olfactory preferences in C. elegans. Cell 91, 161-169.
Troemel, E. R., Sagasti, A. and Bargmann, C. I. (1999). Lateral signaling mediated by axon contact and calcium entry regulates asymmetric odorant receptor expression in C. elegans. Cell 99, 387-398.
Truman, J. W., Taylor, B. J. and Awad, T. A. (1993) Formation of the adult nervous system. In The Development of Drosophila melanogaster Vol II, M. Bate and A. M. Arias, eds. (Cold Spring Harbor Laboratory Press), pp. 1245-1275.
Ugawa, S., Minami, Y., Guo, W., Saishin, Y., Takatsuji, K., Yamamoto, T., Tohyama, M. and Shimada, S. (1998). Receptor that leaves a sour taste in the mouth. Nature 395, 555-556.
Vassar, R., Chao, S. K., Sitcheran, R., Nunez, J. M., Vosshall, L. B. and Axel, R. (1994). Topographic organization of sensory projections to the olfactory bulb. Cell 79, 981-991.
Vassar, R., Ngai, J. and Axel, R. (1993). Spatial segregation of odorant receptor expression in the mammalian olfactory epithelium. Cell 74, 309-318.
Von Heijne, G. (1992). Membrane protein structure prediction, hydrophobicity analysis and the positive-inside rule. Journal of Molecular Biology 225, 487-494.
Vosshall, L. B., Amrein, H., Morozov, P. S., Rzhetsky, A. and Axel, R. (1999). A spatial map of olfactory receptor expression in the Drosophila antenna. Cell 96, 725-736.
Vosshall, L. B., Wong, A. M. and Axel, R. (2000). An Olfactory Sensory Map in the Fly Brain. Cell 102, 147-159.
Wang, F., Nemes, A., Mendelsohn, M. and Axel, R. (1998). Odorant receptors govern the formation of a precise topographic map. Cell 93, 47-60.
Wong, G. T., Gannon, K. S. and Margolskee, R. F. (1996). Transduction of bitter and sweet taste by gustducin. Nature 381, 796-800.

What is claimed is:

1.-56. (canceled)

57. An isolated nucleic acid encoding an insect receptor protein, wherein the nucleic acid encodes a protein comprising the amino acid sequence set forth in SEQ ID NO:24.

58. The isolated nucleic acid of claim 57, wherein the nucleic acid is DNA or RNA.

59. The isolated nucleic acid of claim 58, wherein the DNA is cDNA, genomic DNA or synthetic DNA.

60. A vector which comprises a nucleic acid having encoding an insect receptor protein, wherein the nucleic acid encodes a protein comprising the amino acid sequence set forth in SEQ ID NO:24.

61. The vector of claim 60, wherein the nucleic acid is operatively linked to a regulatory element.

62. The vector of claim 61, wherein the vector is a plasmid.

63. A host vector system for production of a polypeptide having the biological activity of an insect receptor, which comprises the vector of claim 62 and a suitable host.

64. The host vector system of claim 63, wherein the suitable host is a bacterial cell, a yeast cell, an insect cell, or an animal cell.

65. A method for producing a polypeptide having the biological activity of an insect receptor which comprises growing the host vector system of claim 65 under conditions permitting production of the polypeptide.
